(19) United States
(12) Patent Application Publication
(10) Pub. No.: US 2017/0044492 A1
(43) Pub. Date: Feb. 16, 2017

(54) CONVERSION OF SOMATIC CELLS INTO NOCICEPTORS, AND METHODS OF USE THEREOF

(71) Applicants: CHILDERN'S MEDICAL CENTER CORPORATION, Boston, MA (US); PRESIDENT AND FELLOWS OF HARVARD COLLEGE, Quincy, MA (US); THE GENERAL HOSPITAL CORPORATION, Boston, MA (US)

(72) Inventors: Clifford J. Woolf, Newton, MA (US); Brian J. Wainger, Brookline, MA (US); Kevin Eggan, Boston, MA (US); Isaac Chiu, Cambridge, MA (US); Justin Ichida, Los Angeles, CA (US)

(21) Appl. No.: 15/101,814

(22) PCT Filed: Dec. 5, 2014
(86) PCT No.: PCT/US14/68844

§ 371 Date: Jun. 3, 2016

Related U.S. Application Data
(60) Provisional application No. 61/912,939, filed on Dec. 6, 2013.

(51) Int. Cl. C12N 5/0793; A61K 35/30; C12N 15/85

(52) U.S. Cl. CPC C12N 5/062; C12N 15/85; A61K 35/30; C12N 2506/1307; C12N 2510/00

(57) ABSTRACT

The present invention provides methods of transdifferentiation of somatic cells, e.g., a fibroblast, into a nociceptor cell, e.g., an induced nociceptor (iNociceptors) with characteristics of a typical nociceptor cell. The present invention also relates to an isolated population comprising iNociceptors, compositions, their use in the study of cellular and molecular mechanisms of peripheral pain generation and peripheral neuropathy, use in in vitro drug discovery assays, pain research, as their use in the treatment of nociceptive pain related diseases or disorders. In particular, the present invention relates to direct conversion of a somatic cell to an iNociceptor cell having nociceptor characteristics by increasing the protein expression of five nociceptor inducing factors selected from Asc11, Myt11, Isl2, Ngn1, Klf7 in a somatic cell, to convert the fibroblast to an iNociceptors which express the markers of adult nociceptors.

### CLAIM OF PRIORITY

This application claims the benefit of U.S. Provisional Patent Application Ser. No. 61/912,939, filed on Dec. 6, 2013, the entire contents of which are hereby incorporated by reference.

### FEDERALLY SPONSORED RESEARCH OR DEVELOPMENT

This invention was made with Government support under Grant No. R37NS039518 awarded by the National Institutes of Health. The Government has certain rights in the invention.

### TECHNICAL FIELD

This invention relates to methods for transdifferentiation of a somatic cell, e.g., a fibroblast to nociceptors (i.e., nociceptor cells). The present invention also relates to an isolated population comprising induced nociceptors, compositions and their use in use in the study of cellular and molecular mechanisms of peripheral pain generation and peripheral neuropathy, use in in vitro drug discovery assays, pain research, as a therapeutic to reverse disease of, or damage to, the peripheral nervous system (PNS), and their use in the treatment of pain related disorders.

### BACKGROUND

The subjective nature of pain as a human experience confounds the clinical study of pain compared to most other neurological diseases, questions the relevance of animal models and complicates the development of effective treatments⁴². For example, humans homozygous for a non-functioning mutation in the voltage-gated sodium channel NaV1.7 have complete insensitivity to pain without impairment of other peripheral sensory modalities⁴³ while the mouse nociceptor-specific knockout has only a minimal acute pain deficit⁴⁴. Modeling human diseases through derived human cells may reduce artifacts related to heterologous expression and compensation due to knockout or other genetic modification.

Although considerable progress has been made in phenotyping neuropathic pain in patients and in studying the neurobiological mechanisms responsible, this has not yet resulted in the development of novel efficacious therapeutics without adverse effects. One postulated explanation for this is that the efficacy of compounds in preclinical rodent models may not be predictive of analgesia in patients. Another reason is that screens for novel analgesic compounds have almost always used heterologous expression systems typically Chinese hamster ovary (CHO), human embryonic kidney (HEK) or similar cell lines that may not recapitulate the natural molecular environmental architecture and post-translational state of the native target in human neurons. Furthermore, there is a growing realization that target screens (e.g. for a single sodium channel) may be less useful than phenotypic screens, which take advantage of potential drug promiscuity and identify modulators of, say the hyperexcitability of a particular neuron.

Directed differentiation from pluripotent stem cells and lineage reprogramming of somatic cells can be used to derive a wide range of different neuronal subtypes^{1,2}, but the power and potential of each technique relative to the other remain unclear, particularly as optimization of either technique may yet yield more mature and specified cell fates. Certain cell types may be more amenable to generation by one than the other approach, as for pancreatic islet cells derived via lineage reprogramming. Furthermore, the degree of maturity and extent to which endogenous cell phenotypic diversity is captured in derived neurons are unknown.

Lineage specific differentiated stem cells are also valuable research tools for a variety of purposes including in vitro screening assays to identify, confirm, test for specification or delivery of therapeutic molecules to treat lineage specific disease, further elucidation of the complex mechanisms of cell lineage specification and differentiation, and identifying critical biochemical differences between normal and diseased or damaged states which can be further contemplated for use as diagnostic or prognostic markers.

While the known sequence of morphogen exposure and consequent molecular changes in the development of specific neuronal types can guide directed differentiation strategies, the selection of transcription factors for lineage reprogramming remains essentially empirical and uncertain. For example, no single transcription factor has proved essential for driving cell fates in all neuronal reprogramming studies to date, despite the fact that specific factors such as Ascl1 or Ngn2 seem particularly potent in deriving a range of different neuronal subtypes^{4,5}. Alternatively, the specific developmental stage at which a particular transcription factor acts may determine whether that factor facilitates or inhibits the patterning of transdifferentiated neurons when generated in combination with Brn2, Asc11 and Myt11 (abbreviated BAM)^{6,7}.

Nociceptors are the first-order neuron in the pain sensory transduction pathway and play the critical first step in the detection of noxious stimuli (nociception) and in the development of inflammatory and neuropathic pain⁸⁻¹¹. Nociceptor neurons employ a host of highly specific and well-characterized ionotropic receptors and ion channels, including TrpV1, TrpA1, TrpM8 and P2X3 receptors as well as slow, tetrodotoxin (TTX)-resistant sodium channels capable of generating the characteristic broad action potential¹². Efforts to derive nociceptors using a small molecule-based directed differentiation strategy from human neural crest precursors have produced neurons that recreate some but not all of these characteristic receptors and channels¹³.

Mutations in the nociceptor-specific proteins underlie a wide range of pain diseases, ranging from the rare but severe channelopathies such as familial erythromyelalgia¹⁴ to the common small fiber neuropathy, which can occur due to activating mutations in NaV1.7 or NaV1.8¹⁵⁻¹⁷. Nociceptors activate only following intense, potentially damaging stimuli in order to provide a protective warning of imminent tissue damage. However, they also have the remarkable capacity to become sensitized after exposure to inflammatory mediators, thus resulting in a reduced activation threshold so that normally innocuous stimuli generate a pain response. This pain hypersensitivity can play a physiologically useful role in minimizing further injury and promoting healing once damage has occurred; however, such transient heightened activation can also lead to chronic activation and promote the development of pathological chronic pain.

Nociceptor neuron development occurs through dorsalization within the neural tube¹⁸, neural crest induction and migration¹⁹ and finally nociceptor specification within the still-multipotent neural crest lineage²⁰. The generation of nociceptor progenitors expressing the TrkA neurotrophin receptor and post-natal nociceptors expressing TrpV1 requires the basic helix-loop-helix transcription factor Neurogenin1 (Ngn1), which is normally present from approximately days E9-E13 in the embryonic mouse. Although developing nociceptors express multiple Trk-family receptors, maturing nociceptors express only TrkA. Brn3a (POU4F1) promotes Runx1 expression which together with Islet1 and Klf7 maintains TrkA expression in the developing nociceptors21-24. A subset of nociceptors that become peptidergic nociceptors maintain TrkA expression and express calcitonin gene-related peptide (CGRP) and substance P; in developing non-peptidergic nociceptors, most of which bind isolectin B4, the glial cell derived neurtotrophic factor (GDNF) receptor Ret replaces TrkA in a process dependent on Runx1, and the loss of Runx1 markedly reduces TrpV1 expression²².

Understanding the pathology of peripheral sensory neuron diseases, as well as development of treatment modalities, is hindered by the difficulties in obtaining human peripheral sensory neurons. The directed differentiation of embryonic stem cells or somatic stem cells into specified peripheral sensory neurons (i.e., nociceptors), would be an ideal reproducible source of such cells for both research and therapeutic application. Early attempts to derive nociceptors by directed differentiation from embryonic stem cells, has yielded only few immature neurons that did not express the core compliment of functional nociceptor-specific channels and receptors.

Therefore there is a need in the art for a method to produce peripheral sensory neurons, in particular nociceptors, directly from embryonic or somatic stem cells with increased purity and yield.

### SUMMARY

At least in part, the present invention is based on the discovery that just five transcription factors can generate neurons from fibroblasts that possess a nociceptive function by expressing diverse but highly specific elements of the nociceptor signal transduction machinery. By direct comparison between the induced nociceptors and primary adult mouse nociceptors, the inventors demonstrate that the induced neurons (“iNociceptors”) mimic bona fide nociceptors not only with regard to the function of the specific individual receptors and channels, such as TrpA1, TrpM8, P2X7, NaV1.8, Prph and CGRP, but also with regard to the population diversity and overlap of expressed receptors within individual neurons. The inventors show that the induced neurons also model inflammatory sensitization, a critical process that underlies both transient pain hypersensitivity as well as the pathological transition to chronic pain and response to cancer chemotherapeutic agents. Finally, the inventors derive human nociceptive from a patient with a familial neuropathy, familial dysautonomia (FD), and show how the neurons reveal potential disease-relevant phenotypes in vitro.

The present invention relates to compositions and a method for direct reprogramming (i.e. transdifferentiation, or cellular reprogramming) of a fibroblast cell to a cell having characteristics of noxious stimulus-detecting (i.e., nociceptor) neurons. In particular, the present invention relates to a method for direct conversion of a fibroblast cell by increasing the protein expression of five transcription factors, selected from any of Asc11, Myt11, Ngn1, Isl2, KLf7, in the somatic cell. In some aspects, the present invention relates to a method for direct conversion of a fibroblast cell by increasing the protein expression of one or more transcription factors selected from any of Asc11, Myt11, Ngn1, Isl2, KLf7, in the somatic cell.

Accordingly, the present invention relates to methods, compositions and kits for producing induced nociceptors (e.g., a nociceptor cell) from a fibroblast. Other embodiments of the present invention relate to an isolated population of nociceptor cells produced by the methods as disclosed herein, i.e., an isolated population of nociceptors by increasing the protein expression of five transcription, selected from any of Asc11, Myt11, Ngn1, Isl2, KLf7, in a fibroblast cell, and methods of their use. In some embodiments, the present invention relates to an isolated population of nociceptor cells produced by the methods as disclosed herein, i.e., an isolated population of nociceptors by increasing the protein expression of one or more transcription factors selected from any of Asc11, Myt11, Ngn1, Isl2, KLf7, in a fibroblast cell, and methods of their use.

Herein, the inventors have demonstrated that the forced expression of a group of select transcription factors is sufficient to convert mouse and human fibroblasts into induced nociceptors (“iNociceptors”). iNociceptors displayed gene expression signature, electrophysiology, and synaptic functionality, similar to primary tdTomato-positive adult mouse neurons. The inventors have successfully demonstrated that fibroblasts can be converted directly into a specific differentiated and functional nociceptors, referred to herein as “iNociceptors” or “induced nociceptors.”

In some embodiments, induced nociceptors exhibit characteristic of normal nociceptors and can express at least two nociceptor genes selected from the group consisting of TrpA1, TrpM8, P2X7, NaV1.8, Prph and CGRP. In some embodiments, induced nociceptors exhibit characteristic of normal nociceptors, including, for example, sensitization of capsaicin response to inflammatory mediators such as Prostaglandin E2 (PGE2) and/or chemotherapeutic agents (e.g., oxaliplatin).

In one aspect, this disclosure provides method for transdifferentiation of a first somatic cell (e.g., a fibroblast) into a nociceptor cell, the method comprising increasing the protein expression of five nociceptor inducing factors selected from the group consisting of Asc11, Myt11, Ngn1, Isl2, Klf7, or a functional fragment thereof, wherein the nociceptor cell exhibits at least two characteristics of an endogenous nociceptor cell, for example, but not limited to expression of nociceptor specific markers. In some embodiments, the protein expression of Asc11, Myt11, Ngn1, Isl2, or Klf7 are increased in a somatic cell, e.g., a fibroblast.

In some embodiments, an increase in the protein expression of one of the five nociceptor inducing factors (Asc11, Myt11, Ngn1, Isl2, Klf7) can be achieved by contacting a somatic cell, e.g., a fibroblast, with an agent which increases the expression of the nociceptor inducing factor, where an agent can be selected from the group consisting of: a nucleotide sequence, a nucleic acid analogue (e.g., Locked nucleic acid (LNA), modified RNA (modRNA)), a protein, an aptamer and small molecule, ribosome, RNAi agent and peptide-nucleic acid (PNA) and analogues or variants thereof. In some embodiments, protein expression is increased by introducing five nucleic acid sequences encoding the five nociceptor inducing factors (Asc11, Myt11, Ngn1, Isl2, Klf7), or encoding a functional fragment thereof, in the somatic cell, e.g., fibroblast.

In some embodiments, protein expression of Asc11 is increased by introducing a nucleic acid sequence encoding an Asc11 polypeptide comprising SEQ ID NO: 1 (human), or a functional fragment of SEQ ID NO: 1, SEQ ID NO: 11 (murine), or a functional fragment of SEQ ID NO: 13 into the somatic cell, e.g., fibroblast.

In some embodiments, protein expression of Myt11 is increased by introducing a nucleic acid sequence encoding a Myt11 polypeptide comprising SEQ ID NO: 3 (human), or a functional fragment of SEQ ID NO: 3, SEQ ID NO: 13 (murine), or a functional fragment of SEQ ID NO: 13 into the somatic cell, e.g., fibroblast.

In some embodiments, protein expression of Ngn1 is increased by introducing a nucleic acid sequence encoding a Ngn1 polypeptide comprising SEQ ID NO: 5 (human), or a functional fragment of SEQ ID NO: 5, SEQ ID NO: 15 (murine), or a functional fragment of SEQ ID NO: 15 into the somatic cell, e.g., fibroblast.

In some embodiments, protein expression of Isl2 is increased by introducing a nucleic acid sequence encoding an Isl2 polypeptide comprising SEQ ID NO: 7 (human), or a functional fragment of SEQ ID NO: 7, SEQ ID NO: 17 (murine), or a functional fragment of SEQ ID NO: 17 into the somatic cell, e.g., fibroblast.

In some embodiments, protein expression of Klf7 is increased by introducing a nucleic acid sequence encoding a Klf7 polypeptide comprising SEQ ID NO: 9 (human), or a functional fragment of SEQ ID NO: 9, SEQ ID NO: 19 (murine), or a functional fragment of SEQ ID NO: 19 into the somatic cell, e.g., fibroblast.

In some embodiments, a nucleic acid sequence is in a vector, such as a viral vector or a non-viral vector. In some embodiments, the vector is a viral vector comprising a genome that does not integrate into the host cell genome.

In some embodiments, the vector comprises a nucleic acid sequence encoding a Asc11 polypeptide or a functional fragment thereof, and/or comprises a nucleic acid sequence encoding a Myt11 polypeptide or a functional fragment thereof and/or comprises a nucleic acid sequence encoding a Ngn1 polypeptide or a functional fragment thereof, and/or comprises a nucleic acid sequence encoding a Isl2 polypeptide or a functional fragment thereof, and/or comprises a nucleic acid sequence encoding a Klf7 polypeptide or a functional fragment thereof.

In some embodiments, somatic cell, e.g., fibroblast is in vitro. In some embodiments, somatic cell, e.g., fibroblast is ex vivo.

In some embodiments, the somatic cell is a mammalian somatic cell (e.g., a murine, bovine, simian, porcine, equine, ovine, or human cell). The somatic cell can be obtained from a subject. In some embodiments, a subject is a human subject. In some embodiments, the subject has, or is at risk of developing a nociceptive pain (e.g., inflammatory or neuropathic pain, pain accompanying a disease selected from the group consisting of rheumatoid arthritis, rheumatoid spondylitis, osteoarthritis, spondylosis deformans, gouty arthritis, juvenile arthritis, scapulohumeral periarthritis, fibromyalgia, and cervical syndrome; lumbago; lumbago accompanying spondylosis deformans; meralgia paresthetica; pain and tumentia after inflammation, surgery or injury; pain after odontectomy; and cancer pain), a nociceptive pain related disease or disorder (e.g., fibromyalgia (i.e., chronic pain in muscles and soft tissue surrounding joints), arthritis, and other inflammatory diseases of ligaments and tendons), pain or neuropathy after cancer chemotherapy. In some embodiments, a somatic cell, e.g., fibroblast is a mammalian cell, such as a human cell.

Another aspect of the present invention relates to a method for the treatment of a subject with a nociceptive pain related disease or disorder, the method comprising administering a composition comprising an isolated population of iNociceptors produced according to the methods as disclosed herein.

Another aspect of the present invention relates to the use of the isolated population of iNociceptors produced by the methods as disclosed herein for administering to a subject in need thereof.

In some embodiments, iNociceptors can be produced from somatic cells, e.g., fibroblasts obtained from the same subject as the composition is administered to (e.g., autologous induced nociceptors s). In alternative embodiments, the iNociceptors are produced from a donor subject (e.g., allogenic induced nociceptors). In some embodiments, the subject has, or has an increased risk of developing a nociceptive pain related disease or disorder, as disclosed herein.

Another aspect of the present invention relates to kits for producing induced nociceptors as disclosed herein. In some embodiments, a kit comprises a. a nucleic acid sequence encoding a Asc11 polypeptide or a functional fragment thereof, b. a nucleic acid sequence encoding a Myt11 polypeptide or a functional fragment thereof, c. a nucleic acid sequence encoding a Ngn1 polypeptide or a functional fragment thereof, d. a nucleic acid sequence encoding a Isl2 polypeptide or a functional fragment thereof, and e. a nucleic acid sequence encoding a Klf7 polypeptide or a functional fragment thereof. In some embodiments, the kit further comprises instructions for direct conversion of a somatic cell, e.g., fibroblast to an induced nociceptor cell with at least two characteristics of an endogenous nociceptor cell.

The present invention further contemplates uses of the nociceptors generated by a method of the present invention. In one embodiment, the nociceptors are used in in vitro assays to identify compounds that can be used as anti-pain therapeutics or to detect risk of activating or damaging nociceptors—as with cancer chemotherapeutic agents. In one embodiment, the nociceptors are used to study the function of nociceptors. In one embodiment, the nociceptors are used as an in vivo cell replacement therapy in an animal suffering from, or at risk for, damage or disease of the PNS.

In one embodiment, the invention provides a method of screening biological agents, comprising, a) providing: i) a nociceptor, and ii) a test compound b) contacting said nociceptor with said test compound and measuring activation or inhibition of nociceptor function, nociceptor sensitization, nocicepotor survival or growth. In one embodiment, said nociceptor is derived from a somatic cell (e.g., a fibroblast).

In one embodiment, the invention provides a method of evaluating a drug for protecting against neuropathic pain by blocking nociceptor sensitization in response treatment (e.g., treatment with inflammatory mediators such as Prostaglandin E2 (PGE2) or chemotherapeutic agents such as oxaliplatin), comprising, a) providing: i) a nociceptor, and ii) a test compound b) contacting said nociceptor with said test compound and measuring activation or inhibition of nociceptor function, nociceptor sensitization, nocicepotor survival or growth. In one embodiment, said nociceptor is derived from a somatic cell (e.g., a fibroblast).

Another aspect of the present invention relates to methods of identifying agents that alone or in combination with other agents directly convert somatic cell, e.g., fibroblast to an induced nociceptor. In some embodiments, the method includes contacting one or more somatic cell, e.g., fibroblast with one or more test agents (simultaneously or at separate times) and determining the presence of a induced nociceptor comprising at least two characteristics of a at least two characteristics of an endogenous nociceptor cell. The test agents may include, but are not limited to, small molecules, nucleic acids, peptides, polypeptides, immunoglobulins, and oligosaccharides. In some embodiments, the method includes determining the level of expression of one or more of the nociceptor inducing factors selected from the group consisting of: Asc11, Myt11, Ngn1, Isl2, and Klf7. Expression levels can be determined by any means known by one of ordinary skill in the art, for example, by RT-PCR or immunological methods.

The term “transdifferentiation” is used interchangeably herein with the phrase “direct conversion” or “direct reprogramming” and refers to the conversion of one differentiated somatic cell type into a different differentiated somatic cell type without undergoing complete reprogramming to an induced pluripotent stem cell (iPSC) intermediate.

The term “reprogramming” as used herein refers to the process that alters or reverses the differentiation state of a somatic cell. The cell can either be partially or terminally differentiated prior to the reprogramming. Reprogramming encompasses complete reversion of the differentiation state of a somatic cell to a pluripotent cell. Such complete reversal of differentiation produces an induced pluripotent (iPS) cell. A partial reversal of differentiation produces a partially induced pluripotent (PiPS) cell. Reprogramming also encompasses partial reversion of the differentiation state, for example to a multipotent state or to a somatic cell that is neither pluripotent or multipotent, but is a cell that has lost one or more specific characteristics of the differentiated cell from which it arises, e.g. direct reprogramming of a differentiated cell to a different somatic cell type. Reprogramming generally involves alteration, e.g., reversal, of at least some of the heritable patterns of nucleic acid modification (e.g., methylation), chromatin condensation, epigenetic changes, genomic imprinting, etc., that occur during cellular differentiation as a zygote develops into an adult.

The term “pluripotent” as used herein refers to a cell with the capacity, under different conditions, to differentiate to more than one differentiated cell type, and preferably to differentiate to cell types characteristic of all three germ cell layers. Pluripotent cells are characterized primarily by their ability to differentiate to more than one cell type, preferably to all three germ layers, using, for example, a nude mouse teratoma formation assay. Pluripotency is also evidenced by the expression of embryonic stem (ES) cell markers, although the preferred test for pluripotency is the demonstration of the capacity to differentiate into cells of each of the three germ layers.

The term “differentiated cell” is meant any primary cell that is not, in its native form, pluripotent as that term is defined herein. It should be noted that placing many primary cells in culture can lead to some loss of fully differentiated characteristics. However, simply culturing such cells does not, on its own, render them pluripotent. The transition to pluripotency requires a reprogramming stimulus beyond the stimuli that lead to partial loss of differentiated character in culture. Reprogrammed pluripotent cells also have the characteristic of the capacity of extended passaging without loss of growth potential, relative to primary cell parents, which generally have capacity for only a limited number of divisions in culture. Stated another way, the term “differentiated cell” refers to a cell of a more specialized cell type derived from a cell of a less specialized cell type (e.g., a stem cell such as an induced pluripotent stem cell) in a cellular differentiation process.

As used herein, the term “somatic cell” refers to are any cells forming the body of an organism, as opposed to germline cells. In mammals, germline cells (also known as “gametes”) are the spermatozoa and ova which fuse during fertilization to produce a cell called a zygote, from which the entire mammalian embryo develops. Every other cell type in the mammalian body—apart from the sperm and ova, the cells from which they are made (gametocytes) and undifferentiated stem cells—is a somatic cell: internal organs, skin, bones, blood, and connective tissue are all made up of somatic cells. In some embodiments the somatic cell is a “non-embryonic somatic cell”, by which is meant a somatic cell that is not present in or obtained from an embryo and does not result from proliferation of such a cell in vitro. In some embodiments the somatic cell is an “adult somatic cell”, by which is meant a cell that is present in or obtained from an organism other than an embryo or a fetus or results from proliferation of such a cell in vitro. Unless otherwise indicated the methods for direct conversion of a somatic cell, e.g., fibroblast to a induced nociceptor can be performed both in vivo and in vitro (where in vivo is practiced when somatic a somatic cell, e.g., fibroblast are present within a subject, and where in vitro is practiced using isolated somatic a somatic cell, e.g., fibroblast maintained in culture).

As used herein, the term “adult cell” refers to a cell found throughout the body after embryonic development.

As used herein, the term “transcription factor” refers to a protein that binds to specific parts of DNA using DNA binding domains and is part of the system that controls the transfer (or transcription) of genetic information from DNA to RNA.

As used herein, the term “nociceptor” in reference to a cell of the present invention refers to a neuron capable of an action potential and sensing noxious stimulus involved in the perception of pain. Stimuli include, but are not limited to, thermal (heat and cold), mechanical, chemical, and inflammation. Nociceptors are cells expressing specific genes and proteins, such as TrpA1, TRPV1, P2X3, Nav.17, NaV1.8, Prph and CGRP, and comprising a morphology described as two distinct processes with a cell body along an axon-like structure.

As used herein, the term “iNociceptor” or “induced nociceptor” refer to a nociceptor produced by direct conversion from a somatic cell, e.g., a fibroblast.

As used herein, the term “endogenous nociceptor” refers to a nociceptor in vivo or a nociceptor produced by differentiation of an embryonic stem cell into a nociceptor, and exhibiting an adult nociceptor phenotype.

The term “stem cell” as used herein, refers to an undifferentiated cell which is capable of proliferation and giving rise to more progenitor cells having the ability to generate a large number of mother cells that can in turn give rise to differentiated, or differentiable daughter cells. The daughter cells themselves can be induced to proliferate and produce progeny that subsequently differentiate into one or more mature cell types, while also retaining one or more cells with parental developmental potential. The term “stem cell” refers to a subset of progenitors that have the capacity or potential, under particular circumstances, to differentiate to a more specialized or differentiated phenotype, and which retains the capacity, under certain circumstances, to proliferate without substantially differentiating. In one embodiment, the term stem cell refers generally to a naturally occurring mother cell whose descendants (progeny) specialize, often in different directions, by differentiation, e.g., by acquiring completely individual characters, as occurs in progressive diversification of embryonic cells and tissues. Cellular differentiation is a complex process typically occurring through many cell divisions. A differentiated cell may derive from a multipotent cell which itself is derived from a multipotent cell, and so on. While each of these multipotent cells may be considered stem cells, the range of cell types each can give rise to may vary considerably. Some differentiated cells also have the capacity to give rise to cells of greater developmental potential. Such capacity may be natural or may be induced artificially upon treatment with various factors. In many biological instances, stem cells are also “multipotent” because they can produce progeny of more than one distinct cell type, but this is not required for “sternness.” Self-renewal is the other classical part of the stem cell definition, and it is essential as used in this document. In theory, self-renewal can occur by either of two major mechanisms. Stem cells may divide asymmetrically, with one daughter retaining the stem state and the other daughter expressing some distinct other specific function and phenotype. Alternatively, some of the stem cells in a population can divide symmetrically into two stems, thus maintaining some stem cells in the population as a whole, while other cells in the population give rise to differentiated progeny only. Formally, it is possible that cells that begin as stem cells might proceed toward a differentiated phenotype, but then “reverse” and re-express the stem cell phenotype, a term often referred to as “dedifferentiation” or “reprogramming” or “retrodifferentiation” by persons of ordinary skill in the art.

As used herein, the term “nociceptor inducing factor” refers to a gene whose expression, contributes to the direct conversion of a somatic cell, e.g., fibroblast, to a nociceptor which exhibits at least two characteristics of an endogenous nociceptor cell. A nociceptor inducing factor can be, for example, genes encoding human transcription factors Asc11 (SEQ ID NO. 1, encoded by SEQ ID NO: 2), Myt11 (SEQ ID NO. 3, encoded by SEQ ID NO: 4), Ngn1 (SEQ ID NO. 5, encoded by SEQ ID NO: 6), Isl2 (SEQ ID NO. 7, encoded by SEQ ID NO: 8) or Klf7 (SEQ ID NO. 9, encoded by SEQ ID NO: 10). A nociceptor inducing factor can be, for example, genes encoding murine transcription factors Asc11 (SEQ ID NO. 11, encoded by SEQ ID NO: 12), Myt11 (SEQ ID NO. 13, encoded by SEQ ID NO: 14), Ngn1 (SEQ ID NO. 15, encoded by SEQ ID NO: 16), Isl2 (SEQ ID NO. 17, encoded by SEQ ID NO: 18) or Klf7 (SEQ ID NO. 19, encoded by SEQ ID NO: 20.

The term “nociceptor-inducing agent” refers to any agent which increases the protein expression of a nociceptor inducing factor, as that term is described herein. Preferably, a nociceptor-inducing agent increases the expression of a nociceptor inducing factor selected from Asc11, Myt11, Ngn1, Isl2 and Klf7.

The term “agent” as used herein means any compound or substance such as, but not limited to, a small molecule, nucleic acid, polypeptide, peptide, drug, ion, etc. An “agent” can be any chemical, entity or moiety, including without limitation synthetic and naturally-occurring proteinaceous and non-proteinaceous entities. In some embodiments, an agent is nucleic acid, nucleic acid analogues, proteins, antibodies, peptides, aptamers, oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof etc. In certain embodiments, agents are small molecule having a chemical moiety. For example, chemical moieties included unsubstituted or substituted alkyl, aromatic, or heterocyclyl moieties including macrolides, leptomycins and related natural products or analogues thereof. Compounds can be known to have a desired activity and/or property, or can be selected from a library of diverse compounds.

As used herein, the term “proliferation” refers to an increase in cell number.

The term “expression” refers to the cellular processes involved in producing RNA and proteins and as appropriate, secreting proteins, including where applicable, but not limited to, for example, transcription, translation, folding, modification and processing. “Expression products” include RNA transcribed from a gene and polypeptides obtained by translation of mRNA transcribed from a gene.

The term “genetically modified” or “engineered” cell as used herein refers to a cell into which an exogenous nucleic acid has been introduced by a process involving the hand of man (or a descendant of such a cell that has inherited at least a portion of the nucleic acid). The nucleic acid may for example contain a sequence that is exogenous to the cell, it may contain native sequences (i.e., sequences naturally found in the cells) but in a non-naturally occurring arrangement (e.g., a coding region linked to a promoter from a different gene), or altered versions of native sequences, etc. The process of transferring the nucleic into the cell can be achieved by any suitable technique. Suitable techniques include calcium phosphate or lipid-mediated transfection, electroporation, and transduction or infection using a viral vector. In some embodiments the polynucleotide or a portion thereof is integrated into the genome of the cell. The nucleic acid may have subsequently been removed or excised from the genome, provided that such removal or excision results in a detectable alteration in the cell relative to an unmodified but otherwise equivalent cell.

The term “isolated cell” as used herein refers to a cell that has been removed from an organism in which it was originally found or a descendant of such a cell. Optionally the cell has been cultured in vitro, e.g., in the presence of other cells. Optionally the cell is later introduced into a second organism or re-introduced into the organism from which it (or the cell from which it is descended) was isolated.

The term “isolated population” with respect to an isolated population of cells as used herein refers to a population of cells that has been removed and separated from a mixed or heterogeneous population of cells. In some embodiments, an isolated population is a substantially pure population of cells as compared to the heterogeneous population from which the cells were isolated or enriched from.

The term “substantially pure”, with respect to a particular cell population, refers to a population of cells that is at least about 75%, preferably at least about 85%, more preferably at least about 90%, and most preferably at least about 95% pure, with respect to the cells making up a total cell population. Recast, the terms “substantially pure” or “essentially purified”, with regard to a population of iNociceptors, refers to a population of cells that contain fewer than about 20%, more preferably fewer than about 15%, 10%, 8%, 7%, most preferably fewer than about 5%, 4%, 3%, 2%, 1%, or less than 1%, of cells that are not iNociceptors or their progeny as defined by the terms herein. In some embodiments, the present invention encompasses methods to expand a population of iNociceptors, wherein the expanded population of iNociceptors is a substantially pure population of iNociceptors.

As used herein, the term “one or more” includes at least one, more suitably, one, two, three, four, five, six, seven, eight, nine, ten, twenty, fifty, one-hundred, etc., of the item to which “one or more” refers.

The term “statistically significant” or “significantly” refers to statistical significance and generally means a two standard deviation (2SD) below normal, or lower, concentration of the marker. The term refers to statistical evidence that there is a difference. It is defined as the probability of making a decision to reject the null hypothesis when the null hypothesis is actually true. The decision is often made using the p-value.

As used herein, the term “DNA” is defined as deoxyribonucleic acid.

As used herein, the term “gene” used herein can be a genomic gene comprising transcriptional and/or translational regulatory sequences and/or a coding region and/or non-translated sequences (e.g., introns, 5′- and 3′-untranslated sequences and regulatory sequences). The coding region of a gene can be a nucleotide sequence coding for an amino acid sequence or a functional RNA, such as tRNA, rRNA, catalytic RNA, siRNA, miRNA and antisense RNA. A gene can also be an mRNA or cDNA corresponding to the coding regions (e.g. exons and miRNA) optionally comprising 5′- or 3′ untranslated sequences linked thereto. A gene can also be an amplified nucleic acid molecule produced in vitro comprising all or a part of the coding region and/or 5′- or 3′-untranslated sequences linked thereto.

The term “polynucleotide” is used herein interchangeably with “nucleic acid” to indicate a polymer of nucleosides. Typically a polynucleotide of this invention is composed of nucleosides that are naturally found in DNA or RNA (e.g., adenosine, thymidine, guanosine, cytidine, uridine, deoxyadenosine, deoxythymidine, deoxyguanosine, and deoxycytidine) joined by phosphodiester bonds. However the term encompasses molecules comprising nucleosides or nucleoside analogs containing chemically or biologically modified bases, modified backbones, etc., whether or not found in naturally occurring nucleic acids, and such molecules may be preferred for certain applications. Where this application refers to a polynucleotide it is understood that both DNA, RNA, and in each case both single- and double-stranded forms (and complements of each single-stranded molecule) are provided. “Polynucleotide sequence” as used herein can refer to the polynucleotide material itself and/or to the sequence information (i.e. the succession of letters used as abbreviations for bases) that biochemically characterizes a specific nucleic acid. A polynucleotide sequence presented herein is presented in a 5′ to 3′ direction unless otherwise indicated. The terms “nucleic acid” can also refer to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides. The terms “polynucleotide sequence” and “nucleotide sequence” are also used interchangeably herein. Nucleic acids can be single stranded or double stranded, or can contain portions of both double stranded and single stranded sequence. The nucleic acid can be DNA, both genomic and cDNA, RNA, or a hybrid, where the nucleic acid can contain combinations of deoxyribo- and ribonucleotides, and combinations of bases including uracil, adenine, thymine, cytosine, guanine, inosine, xanthine hypoxanthine, isocytosine and isoguanine. Nucleic acids can be obtained by chemical synthesis methods or by recombinant methods.

The terms “polypeptide” as used herein refers to a polymer of amino acids. The terms “protein” and “polypeptide” are used interchangeably herein. A peptide is a relatively short polypeptide, typically between about 2 and 60 amino acids in length. Polypeptides used herein typically contain amino acids such as the 20 L-amino acids that are most commonly found in proteins. However, other amino acids and/or amino acid analogs known in the art can be used. One or more of the amino acids in a polypeptide may be modified, for example, by the addition of a chemical entity such as a carbohydrate group, a phosphate group, a fatty acid group, a linker for conjugation, functionalization, etc. A polypeptide that has a non-polypeptide moiety covalently or non-covalently associated therewith is still considered a “polypeptide”. Exemplary modifications include glycosylation and palmitoylation.

Polypeptides may be purified from natural sources, produced using recombinant DNA technology, synthesized through chemical means such as conventional solid phase peptide synthesis, etc. The term “polypeptide sequence” or “amino acid sequence” as used herein can refer to the polypeptide material itself and/or to the sequence information (i.e., the succession of letters or three letter codes used as abbreviations for amino acid names) that biochemically characterizes a polypeptide. A polypeptide sequence presented herein is presented in an N-terminal to C-terminal direction unless otherwise indicated.

The terms “polypeptide variant” refers to any polypeptide differing from a naturally occurring polypeptide by amino acid insertion(s), deletion(s), and/or substitution(s). Variants may be naturally occurring or created using, e g., recombinant DNA techniques or chemical synthesis. In some embodiments amino acid “substitutions” are the result of replacing one amino acid with another amino acid having similar structural and/or chemical properties, i.e., conservative amino acid replacements. “Conservative” amino acid substitutions may be made on the basis of similarity in any of a variety or properties such as side chain size, polarity, charge, solubility, hydrophobicity, hydrophilicity, and/or amphipathicity of the residues involved. For example, the non-polar (hydrophobic) amino acids include alanine, leucine, isoleucine, valine, glycine, proline, phenylalanine, tryptophan and methionine. The polar (hydrophilic), neutral amino acids include serine, threonine, cysteine, tyrosine, asparagine, and glutamine. The positively charged (basic) amino acids include arginine, lysine and histidine. The negatively charged (acidic) amino acids include aspartic acid and glutamic acid. Insertions or deletions may range in size from about 1 to 20 amino acids, e.g., 1 to 10 amino acids. In some instances larger domains may be removed without substantially affecting function. In certain embodiments of the invention the sequence of a variant can be obtained by making no more than a total of 5, 10, 15, or 20 amino acid additions, deletions, or substitutions to the sequence of a naturally occurring enzyme. In some embodiments not more than 1%, 5%, 10%, 15% or 20% of the amino acids in a polypeptide are insertions, deletions, or substitutions relative to the original polypeptide. Guidance in determining which amino acid residues may be replaced, added, or deleted without eliminating or substantially reducing activities of interest, may be obtained by comparing the sequence of the particular polypeptide with that of homologous polypeptides (e.g., from other organisms) and minimizing the number of amino acid sequence changes made in regions of high homology (conserved regions) or by replacing amino acids with those found in homologous sequences since amino acid residues that are conserved among various species are more likely to be important for activity than amino acids that are not conserved.

By “amino acid sequences substantially homologous” to a particular amino acid sequence (e.g. Asc11, Myt11, Ngn1, Isl2 and Klf7) is meant polypeptides that include one or more additional amino acids, deletions of amino acids, or substitutions in the amino acid sequence of Asc11, Myt11, Ngn1, Isl2 and Klf7 without appreciable loss of functional activity as compared to wild-type Asc11, Myt11, Ngn1, Isl2 and Klf7 polypeptides in terms of the ability to produce iNociceptors from a somatic cell, e.g., fibroblast. For example, the deletion can consist of amino acids that are not essential to the presently defined differentiating activity and the substitution(s) can be conservative (i.e., basic, hydrophilic, or hydrophobic amino acids substituted for the same). Thus, it is understood that, where desired, modifications and changes may be made in the amino acid sequence of Asc11, Myt11, Ngn1, Isl2 and Klf7, and a protein having like characteristics still obtained. It is thus contemplated that various changes may be made in the amino acid sequence of the Asc11, Myt11, Ngn1, Isl2 and Klf7 amino acid sequence (or underlying nucleic acid sequence) without appreciable loss of biological utility or activity and possibly with an increase in such utility or activity. In some embodiments, the amino acid sequences substantially homologous to a particular amino acid sequence are at least 70%, e.g., 75%, 80%85%, 90%, 95% or another percent from 70% to 100%, in integers thereof, identical to the particular amino acid sequence.

The term “lineages” as used herein describes a cell with a common ancestry or cells with a common developmental fate. In the context of a cell that is of “neuronal linage” this means the cell can differentiate along the neuronal lineage restricted pathways.

The terms “decrease”, “reduced”, “reduction”, “decrease” or “inhibit” are all used herein generally to mean a decrease by a statistically significant amount. However, for avoidance of doubt, ““reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “vector” refers to a carrier DNA molecule into which a DNA sequence can be inserted for introduction into a host cell. Preferred vectors are those capable of autonomous replication and/or expression of nucleic acids to which they are linked. Vectors capable of directing the expression of genes to which they are operatively linked are referred to herein as “expression vectors”. Thus, an “expression vector” is a specialized vector that contains the necessary regulatory regions needed for expression of a gene of interest in a host cell. In some embodiments the gene of interest is operably linked to another sequence in the vector. Vectors can be viral vectors or non-viral vectors. Should viral vectors be used, it is preferred the viral vectors are replication defective, which can be achieved for example by removing all viral nucleic acids that encode for replication. A replication defective viral vector will still retain its infective properties and enters the cells in a similar manner as a replicating adenoviral vector, however once admitted to the cell a replication defective viral vector does not reproduce or multiply. Vectors also encompass liposomes and nanoparticles and other means to deliver DNA molecule to a cell.

The term “viral vectors” refers to the use of viruses, or virus-associated vectors as carriers of a nucleic acid construct into a cell. Constructs may be integrated and packaged into non-replicating, defective viral genomes like Adenovirus, Adeno-associated virus (AAV), or Herpes simplex virus (HSV) or others, including retroviral and lentiviral vectors, for infection or transduction into cells. The vector may or may not be incorporated into the cell's genome. The constructs may include viral sequences for transfection, if desired. Alternatively, the construct may be incorporated into vectors capable of episomal replication, e.g. EPV and EBV vectors.

As used herein, the term “adenovirus” refers to a virus of the family Adenovirida. Adenoviruses are medium-sized (90-100 nm), nonenveloped (naked) icosahedral viruses composed of a nucleocapsid and a double-stranded linear DNA genome.

As used herein, the term “non-integrating viral vector” refers to a viral vector that does not integrate into the host genome; the expression of the gene delivered by the viral vector is temporary. Since there is little to no integration into the host genome, non-integrating viral vectors have the advantage of not producing DNA mutations by inserting at a random point in the genome. For example, a non-integrating viral vector remains extra-chromosomal and does not insert its genes into the host genome, potentially disrupting the expression of endogenous genes. Non-integrating viral vectors can include, but are not limited to, the following: adenovirus, alphavirus, picornavirus, and vaccinia virus. These viral vectors are “non-integrating” viral vectors as the term is used herein, despite the possibility that any of them may, in some rare circumstances, integrate viral nucleic acid into a host cell's genome. What is critical is that the viral vectors used in the methods described herein do not, as a rule or as a primary part of their life cycle under the conditions employed, integrate their nucleic acid into a host cell's genome. It goes without saying that an iNociceptor cell generated by a non-integrating viral vector will not be administered to a subject unless it and its progeny are free from viral remnants.

A “marker” as used herein is used to describe the characteristics and/or phenotype of a cell. Markers can be used for selection of cells comprising characteristics of interests. Markers will vary with specific cells. Markers are characteristics, whether morphological, functional or biochemical (enzymatic) characteristics of the cell of a particular cell type, or molecules expressed by the cell type. Preferably, such markers are proteins, and more preferably, possess an epitope for antibodies or other binding molecules available in the art. However, a marker may consist of any molecule found in a cell including, but not limited to, proteins (peptides and polypeptides), lipids, polysaccharides, nucleic acids and steroids. Examples of morphological characteristics or traits include, but are not limited to, shape, size, and nuclear to cytoplasmic ratio. Examples of functional characteristics or traits include, but are not limited to, the ability to adhere to particular substrates, ability to incorporate or exclude particular dyes, ability to migrate under particular conditions, and the ability to differentiate along particular lineages. Markers may be detected by any method available to one of skill in the art. Markers can also be the absence of a morphological characteristic or absence of proteins, lipids etc. Markers can be a combination of a panel of unique characteristics of the presence and absence of polypeptides and other morphological characteristics.

The terms “treat”, “treating”, “treatment”, etc., as applied to an isolated cell, include subjecting the cell to any kind of process or condition or performing any kind of manipulation or procedure on the cell. As applied to a subject, the term “treating” refer to providing medical or surgical attention, care, or management to an individual. The individual is usually ill or injured, or at increased risk of becoming ill relative to an average member of the population and in need of such attention, care, or management.

In some embodiments, the term “treating” and “treatment” refers to administering to a subject an effective amount of a composition, e.g., a composition comprising iNociceptors or their differentiated progeny so that the subject as a reduction in at least one symptom of the disease or an improvement in the disease, for example, beneficial or desired clinical results. For purposes of this invention, beneficial or desired clinical results include, but are not limited to, alleviation of one or more symptoms, diminishment of extent of disease, stabilized (i.e., not worsening) state of disease, delay or slowing of disease progression, amelioration or palliation of the disease state, and remission (whether partial or total), whether detectable or undetectable. Treating can refer to prolonging survival as compared to expected survival if not receiving treatment. Thus, one of skill in the art realizes that a treatment may improve the disease condition, but may not be a complete cure for the disease. In some embodiments, treatment can be “prophylaxic treatment, where the subject is administered a composition as disclosed herein (e.g., a population of iNociceptors or their progeny) to a subject at risk of developing a nociceptive pain related disease as disclosed herein. In some embodiments, treatment is “effective” if the progression of a disease is reduced or halted.

As used herein, the terms “administering,” “introducing” and “transplanting” are used interchangeably in the context of the placement of iNociceptors of the invention into a subject, by a method or route which results in at least partial localization of the iNociceptor at a desired site. In some embodiments, the iNociceptors can be placed directly in the spinal cord or in the cerebellum, or alternatively be administered by any appropriate route which results in delivery to a desired location in the subject where at least a portion of the cells or components of the cells remain viable. The period of viability of the cells after administration to a subject can be as short as a few hours, e. g. twenty-four hours, to a few days, to as long as several or more years.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is a linear reprogramming schematic demonstrating MEFs isolated from TrpV1+/−::tdTomato+/− mice, transduced with candidate transcription factors, and detection of tdTomato-positive induced neurons.

FIGS. 2a-f are a series of photographs showing TRPV1-tdTomato+ neurons generated from fibroblasts by forced expression of selected transcription factors. Single-factor drop out experiments were performed to identify inhibitory and essential factors. (a) retroviral transduction of MEFs with a combination of 12 transcription factors produces a few tdTomato-positive, Tuj1-positive neurons. (b, c) Removing Runx1 (b), or Brn3a (c) from the 12 factors increases the number of tdTomato-positive cells. (d-f) Removing Asc11 (d), Myt11 (e), or Klf7 (f) results in a decrease in TdTomato-positive cells.

FIGS. 3a-g are a series of photographs showing TRPV1-tdTomato+ neurons generated from fibroblasts by forced expression of selected transcription factors. A combination of five transcription factors results in optimal nociceptor production. (a) Minimal tdTomato, Tuj1-positive neurons are produced by the combination of six factors (6 TFs): Brn2, Asc11, Myt11, Ngn1, Isl2 and Klf7. (b) Removal of Brn2 markedly increases the number of tdTomato-, Tuj1-positive neurons. (c-g) Omission of Asc11 (c), Myt11 (d), Ngn1 (e), Isl2 (f) or Klf7 (g) from the six factors disrupts the generation of nociceptor neurons. Representative images for each transcription factor drop out were taken from n=4 wells from two separate transductions. Scale bars: 100 μm.

FIG. 4 is a graph demonstrating quantification of the number of tdTomato neurons per well for the single factor drop-out studies (n=4). Removal of Brn2 markedly increases the number of tdTomato, Tuj1-positive neurons. Omission of Asc11, Myt11, Ngn1, Isl2 or Klf7 from the six factors disrupts the generation of nociceptor neurons.

FIGS. 5a-d are a series of photographs and a graph showing TRPV1-tdTomato+ neurons generated from fibroblasts by forced expression of selected combinations of transcription factors. Alternative factor combinations generate low number of tdTomato, Tuj1-positive neurons. (a) Ngn1 alone produces a small number of tdTomato, Tuj1-positive cells. (b) the BAM factors produce large numbers of Tuj1-positive cells, a few of which are tdTomato-positive. (c) BAM factors and Ngn1 produce tdTomato, Tuj1-positive neurons, but much less efficiently than the five factors (see FIG. 3a-g and FIG. 4). Representative images for each transcription factor combination study were taken from n=4 wells from 2 separate transductions. Scale bars: 100 nm. (d) Quantification of the number of tdTomato-positive neurons per well.

FIGS. 6a-f are a series of photographs showing TRPV1-tdTomato+ neurons generated from fibroblasts by forced expression of selected combinations of transcription factors. (a) Transduction of MEFs with all 5 factors (Asc11, Myt11, Ngn1, Isl2, and Klf7) efficiently produce tdTomato, Tuj1-positive neurons. (b-f) Removal of Asc11 (b), Myt11 (c), Ngn1 (d), Isl2 (e), or Klf7 (f) dramatically reduces the number of tdTomato, Tuj1-positive neurons. Representative images of each transcription factor drop out were selected from n=4 wells from two separate transductions. Scale bars: 100 μm.

FIGS. 7a-d are a series of photographs demonstrating induced nociceptors (iNoc) express characteristic nociceptor genes. (a, b) Tuj1 (a) and TrpV1 (b) expression in fibroblast-derived nociceptor neurons. (c) Most induced nociceptors stain for the C-fiber marker peripherin. (d) A number of induced nociceptors expressed the peptidergic-marker CGRP. (e) A small number of induced nociceptors expressed the intermediate filament marker NF200 found in myelinated fibers. Representative images were selected from immunostaining that was repeated in n=4 wells from two independent transductions. Scale bars represent 100 μm.

FIG. 8 is a graph demonstrating quantitative RT-PCR data showing expression levels of nociceptor-specific genes in 50 picked tdTomato-positive primary adult mouse nociceptors (DRGs, black circles) and 50 picked tdTomato-positive induced nociceptors (red circles), relative to their levels in MEFs, from a minimum of two independent biological replicates (biological replicates represented as independent circles

FIGS. 9a-d are a series of graphs demonstrating RT qPCR data showing induced nociceptors express VaV1.8 and characteristic MEF genes. (a-c) qPCR data showing curves for Gapdh and NaV1.8 for MEFs (a), tdTomato-positive FACs-sorted primary adult mouse nociceptors (DRGs) (b), and tdTomato-positive induced nociceptors (iNoc) (c). (d) qPCR data showing expression levels of characteristic MEF genes in FACs sorted tdTomato-positive primary nociceptors (solid gray bars) and tdTomato-positive induced nociceptors (striped gray bars), relative to their levels in MEFs.

FIG. 10a-c are a series of graphs and a diagram demonstrating induced neurons respond to different Trp channel agonists. (a) Sample calcium imaging responses to sequential application of menthol (250 μM), mustard oil (100 μM), capsaicin (1 μM), and potassium chloride (40 mM) in a single dish of induced tdTomato-positive derived nociceptors. Traces are representative recordings from n=227 tdTomato-positive/KCl-responding cells cultured in 19 dishes from 3 independent transductions (b) Venn diagram showing subgroups of tdTomato-positive cells that responded to KCl (40 mM, grey), capsaicin (Cap, 1 μM, red, 39%), mustard oil (MO, 100 μM, lower small circle, green, 9%) and menthol (ME, 250 μM, upper small partial circle, blue, 3%; note that no tdTomato-positive cells respond to menthol alone) (c) Sample electrodes from extracellular multi-electrode array recordings of induced neurons before (left) and after (right) the application of capsaicin (1 μM, upper) and mustard oil (100 μM, lower). Sample recordings for each agonist are indicative of results from three experiments across two independent transductions, in which all replicates showed an increase in firing after agonist application.

FIGS. 11a-c are a series of graphs and a diagram demonstrating sample calcium imaging responses to Trp agonists in induced and adult primary nociceptors. (a) Examples of tdTomato-negative induced neurons that responded to menthol (250 μM) but not mustard oil (100 μM). (b) Sample calcium imaging responses from a single field of adult primary tdTomato-positive nociceptors. (c) Venn diagram showing subgroups of tdTomato-positive cells that responded to KCl (40 mM), capsaicin (Cap), mustard oil (MO, lower small circle) and menthol (ME, upper small circle). No cells responded to mustard oil and menthol without responding to capsaicin.

FIGS. 12a-h are a series of graphs demonstrating whole-cell patch clamp of induced nociceptors. (a) Current recording in response to treatment with 1 μM capsaicin (6/11 induced neuron responded). (b) Current recording following the application of 30 μM α, β-methylene-ATP (8/16 induced neurons responded). (c) Inward currents following step depolarization before (left) and after (right) the application of 300 nM tetrodotoxin (TTX) (14/15 induced neurons had TTX-resistant sodium currents greater than 50 pA). (d) Action potential firing elicited by depolarizing current in the presence of 300 nM TTX (7/12 cells fired single TTX-resistant action potentials with peak greater than 0 mV). (e, f) Examples of individual action potentials (e) and trains (f) elicited from induced nociceptors (iNoc), tdTomato-positive primary adult nociceptors (Primary Noc) and tdTomato-negative primary adult non-nociceptors (Primary Non-Noc) (12/13 induced neurons fired tonically; 6/13 had width at half-maximal amplitude greater than 3 ms). (g) Examples of sag depolarizations in response to hyperpolarizing current injections in induced nociceptors (11/17 induced neurons produced a sag depolarization). (h) CGRP was released from induced nociceptors (5F), but not BAM-derived neurons, in response to KCl (80 mM), but not vehicle. Mean (s.e.m.) for induced nociceptors and BAM following KCl stimulation were 390.4 (52.5) and 10.3 (2.6) pg ml-1 (n=4, Mann-Whitney U test, P=0.03).

FIGS. 13a-c are a series of graphs demonstrating sensitization of induced nociceptors treated with the inflammatory mediator PGE2. (a) Sample calcium imaging recordings of induced nociceptors treated with 300 nM capsaicin before and after treatment with 1 μM PGE2 from recording of n=41 tdTomato-positive/KCl-responding cells. (b) Plot of individual and mean response amplitudes for initial and PGE2-sensitized capsaicin treatments. (c) Plot of initial versus PGE2-sensitized capsaicin response amplitudes for individual induced neurons. (d) Sample traces from extracellular MEA recordings of induced neurons in response to 300 nM capsaicin following a 10-min exposure to vehicle control (n=5 MEAs) or oxaliplatin (50 μM, n=4 MEAs) on induced neurons from two separate transductions. (e) Quantification of spikes per minute from induced nociceptors in response to capsaicin alone (control) and capsaicin following oxaliplatin treatment. Error bars represent±s.e.m.

FIGS. 14a-e are photographs and graphs demonstrating Tuj1 and peripherin expression in HC-derived nociceptor neurons and FD-derived nociceptor neurons.

Human fibroblast-derived neurons for human disease modeling. (a) Low magnification of Tuj1 (left) and peripherin (Prph, right) staining of HC-derived neurons. Scale bars represent 500 μm. (b) High magnification of Tuj1 staining of HC-derived neurons. Scale bar represents 100 μm. (c) NF200-positive cell derived from HC fibroblasts. (d) Current recording of an action potential train from a HC-derived neuron (17 of 33 induced neurons with peak Na current >500 pA fired at least one action potential with peak greater than 0 mV). (e) Total (left) and TTX-resistant (middle) sodium currents from a single HC-derived neuron. Right, persistent TTX-resistant sodium current recordings from a separate HC-derived neuron characteristic of NaV1.9. (f) RT-PCR for IKBKAP and GAPDH from single human induced neurons (left) and single human fibroblasts (right) showed normal (arrow) and abnormally spliced (arrowhead) transcripts. Full-length gels are represented in FIGS. 20a-b)(g) Low magnification of Tuj1 (left) and peripherin (right) staining of neurons derived from a patient with FD. Scale bars represent 500 μm. (h) High magnification of Tuj1 staining of FD-derived neurons. Scale bar represents 100 μm. For all images, representative images were selected from human neurons generated in n=6 wells from three separate transductions.

FIGS. 15a-c are a series of graphs demonstrating (a) quantification of Tuj1-positive neurons in HC- and FD-derived nociceptors (random intercept mixed-effects model, P=0.26). (b) Neurite outgrowth per cell for HC- and FD-derived Tuj1-positive nociceptors (random intercept mixed effects model, P=0.012). (c) Number of branches per cell for HC- and FD-derived Tuj1-positive nociceptors (random intercept mixed effects model P=0.017). For a,c, images were analyzed from three pairs of age-matched HC and FD patient lines from each of three separate transductions (n=20 wells per line). Error bars represent ±s.e.m.

FIGS. 16a-c are photographs demonstrating staining of MEFs for neuronal precursor markers using antibodies to Nestin, Sox1, and Ki67, as well as for neuron-specific class III β-tubulin (Tuj1). (a) Representative images of TrpV1-Cre::tdTomato MEFs reveal lack of staining for Nestin (upper), Sox1 (middle), and Ki67 (lower). Bright field images confirm the presence of cells in the field of view. (b) Positive-control rat neural stem cells stain positive for Nestin (upper), Sox1 (middle), and Ki67 (lower). (c) TrpV1-Cre::tdTomato MEFs reveal lack of staining for Tuj1. (d) Positive-control TrpV1-Cre::tdTomato induced nociceptors (iNoc) stain positive for Tuj1. Representative images of induced neurons were selected from n=4 wells per antibody from 2 separate transductions and from rat neural stem cells from n=4 wells from one plating. Scale bars: 100 μm.

FIGS. 17a-g are (a,b) representative images of TrpV1-Cre::tdTomato iNoc (a) revealing lack of staining for smooth muscle actin (SMA), compared to positive-control C2C12 mouse myoblast cells (b). (c-g) BAM-factor derived, non-subtype specific induced neurons express the pan-neuronal marker Tuj1 (c), but not the nociceptor-specific markers TrpV1 (d), Prph (e), CGRP (f) or NF200 (g). Representative image for SMA in induced nociceptors was selected from n=4 wells from 2 independent transductions and representative image for SMA in myoblasts was selected from n=4 wells from 1 plating of myoblasts. Representative images for BAM-derived neurons were selected from immunostaining of n=6 wells from 3 independent transductions. Scale bars: 100 μm.

FIG. 18 is a graph demonstrating CGRP release from primary DRGs in response to KCl and capsaicin. CGRP ELISA reveals a dose-dependent increase in CGRP release from in vitro primary DRGs in response to increasing concentrations of KCl compared to capsaicin (100 nM).

FIGS. 19a-d show (a,b) representative low-mag images of human iNoc derived with 5 factors (without NeuroD1), stained with Tuj1. (c,d) Representative low-mag images of human iNoc derived with 6 factors (including NeuroD1), stained with Tuj1. Representative images for human induced neurons with or without NeuroD1 were selected from immunostaining that was repeated in n=6 wells from one transduction. Scale bars: 500 μm. The reprogramming efficiency was greater without NeuroD1 than with NeuroD1 (20.7±1.4 cells per field without NeuroD1; 9.7±1.1 cells per field with NeuroD1, n=6 wells/group; t-test p=1.0×10⁻⁴).

FIGS. 20a-b are single cell RT-PCR full gels showing RT-PCR for IKBKAP and GAPDH from single human induced neurons (a) and single human fibroblasts (b) show normal (arrow) and abnormally spliced (arrowhead) transcripts.

### DETAILED DESCRIPTION

Pain is a self-conscious combined sensation associated with actual or potential tissue damage and emotional response there to, which come in many varieties. In addition, pain is broadly classified into somatogenic pain and psychogenic pain, and the former is classified into nociceptive pain and neuropathic pain. Nociceptive pain is caused by external stimuli or endogenous pathology. Nociceptive pain is divided into acute diseases and chronic diseases, but is mostly acute pain which disappears when the underlying disease is cured, which acts as a biological signal for disorders. Neuropathic pain is chronic pain caused by dysfunction of nervous systems in peripheral or central nerves, which includes diabetes-derived pain, nerve compression, spinal injuries and the like. Psychogenic pain is organically unexplained chronic pain which is caused by a mental disorder rather than a physical disorder and includes chronic headaches, unknown stomach aches and the like. In such a pain, chronic (constant) pain is a target to be treated due to the serious suffering of the patients. In particular, regarding chronic pain accompanying arthritis, diabetes, cancers and the like, there is a need for treatment of the underlying disease but also for treatment of the associated pain, but conventional analgesic agents have unsatisfactory efficacy and safety.

Nociceptive pain, as used herein and as defined in the literature, is pain that results from tissue damage, and wherein there is not any substantial nerve damage. Instead, intact neurons report the tissue damage, and pain is experienced. Nociceptive pain can be cutaneous pain, somatic pain or visceral pain. Nociceptive pain can be experienced as sharp, dull or aching. In addition, nociceptive pain can be either acute or chronic. Encompassed within the category of nociceptive pain are fibromyalgia (i.e., chronic pain in muscles and soft tissue surrounding joints), arthritis, and other inflammatory diseases of ligaments and tendons as well as the pain induced by exposure to cancer chemotherapeutic agents.

In nociceptive pain, tissue associated with joints such as bones or cartilage is involved in the onset of chronic pain accompanying arthritis. Cartilage tissue is a tissue composed of cartilage cells and cartilage substrates, which forms skeletal systems with bones. Osteoarthritis is a disease in which articular cartilage is chronically worn or lost, and the cartilage is deformed. Osteoarthritis includes two kinds of osteoarthritis (i.e., primary and secondary osteoarthritis). Primary osteoarthritis is caused by factors such as muscular degeneration, obesity or mechanical stress and secondary osteoarthritis is caused by clear factors such as injury or diseases. Rheumatoid arthritis is a disease which is characterized by unexplained chronic arthritis and causes inflammation of articular synovium, if progressed, destruction of cartilage and bones or articular deformation.

Other forms of nociceptive pain and/or disease induced pain that are not associated with substantial nerve damage include anoxic, Raynauds, myo facial, autoimmune, ischemic, as well as certain types of nociceptive pain induced by neuropathic processes, diffuse nonorganic pain, non-organic back pain, trigeminal pain, connective tissue diseases, diabetic neuropathy, shingles pain syndrome, fibromyalgia, ligament sprain, arthritis, headache, migraine pain, tendon pain, ligament pain, arachnoiditis-induced pain, chronic pain, endometriosis, and nerve pain associated with diabetes or shingles.

The present inventors have developed methods for transdifferentiation of a somatic cell into a nociceptor cell. The process of altering the cell phenotype of a differentiated cell (i.e. a first cell), e.g., altering the phenotype of a somatic cell to a differentiated cell of a different phenotype (i.e. a second cell) without the first differentiated cell being completely reprogrammed to a less differentiated phenotype intermediate is referred to as “direct reprogramming” or “transdifferentiation”. Stated another way, cells of one type can be converted to another type in a process by what is commonly referred to in the art as transdifferentiation, cellular reprogramming or lineage reprogramming.

Transdifferentiation encompasses a process of switching the phenotype of a first differentiated cell to the phenotype of a second different differentiated cell, without the complete reversal of the differentiation state of the somatic cell, and is different from “reprogramming a cell to a pluripotent state” which typically refers to a process which partially or completely reverses the differentiation state of a somatic cell to a cell with a stem cell-like phenotype, e.g., to an induced pluripotent stem cell (iPSC).

As disclosed herein, the present invention relates to methods for transdifferentiation of a somatic cell (e.g., fibroblasts) into a nociceptor cell, referred to as “induced nociceptors” or “iNociceptors.” The methods contemplated herein comprise increasing the protein expression of five nociceptor inducing factors selected from the group consisting of Asc11, Myt11, Ngn1, Isl2, Klf7, or a functional fragment thereof, wherein the nociceptor cell exhibits at least two characteristics of an endogenous nociceptor cell.

As disclosed herein, the present invention relates to methods for transdifferentiation of a somatic cell (e.g., fibroblasts) into a nociceptor cell, the methods comprising increasing the protein expression of one or more nociceptor inducing factors including Asc11, Myt11, Ngn1, Isl2, Klf7, or a functional fragment thereof, wherein the nociceptor cell exhibits at least two characteristics of an endogenous nociceptor cell. In some embodiments, the methods disclosed herein further comprise increasing the protein expression of one or more nociceptor inducing factors including Drgx, Ebf1, Etv1, Isl2, Pknox2, Brn3a, Runx1, Tlx3, or a functional fragment thereof.

In certain embodiments of the invention, the transdifferentiation of a somatic cell, e.g., fibroblast causes the somatic cell to assume a nociceptor like state, without being completely reprogrammed to a pluripotent state prior to becoming an iNociceptor.

In some embodiments, the methods and compositions of the present invention can be practiced on somatic cells that are fully differentiated and/or restricted to giving rise only to cells of that particular type. The somatic cells can be either partially or terminally differentiated prior to direct conversion to iNociceptors. In some embodiments, somatic cells which are trandifferentiated into iNociceptors are fibroblast cells.

In some embodiments, the population of a somatic cell, e.g., fibroblast is a substantially pure population of fibroblasts. In some embodiments, a population of a somatic cell, e.g., fibroblast is a population of somatic cells or differentiated cells. In some embodiments, the population of a somatic cell, e.g., fibroblast are substantially free or devoid of embryonic stem cells or pluripotent cells or iPS cells.

In some embodiments, a somatic cell, e.g., fibroblast is genetically modified. In some embodiments, the somatic cell, e.g., fibroblast comprises one or more nucleic acid sequences encoding the proteins of five induced nociceptor factors selected from Asc11, Myt11, Ngn1, and Isl2, Klf7, as shown in Table 1.

TABLE 1

| Nociceptor | | | | |
|---|---|---|---|---|
| Inducing | Human | Human | Murine | Murine |
| Factor | amino acid | nucleic acid | amino acid | nucleic acid |
| Ascl1 | NP_004307.2 | NM_004316.3 | NP_032579.2 | NM_008553.4 |
| | (SEQ ID NO: 1) | (SEQ ID NO: 2) | (SEQ ID NO: 11) | (SEQ ID NO: 12) |
| Mythl1 | XP_005264742.1 | XM_005264685 | NP_001087244.1 | NM_001093775.1 |
| | (SEQ ID NO: 3) | (SEQ ID NO: 4) | (SEQ ID NO: 13) | (SEQ ID NO: 14) |
| Neurog1 | NP_006152.2 | NM_006161.2 | NP_035026.1 | NM_010896.2 |
| (Ngn1) | (SEQ ID NO: 5) | (SEQ ID NO: 6) | (SEQ ID NO: 15) | (SEQ ID NO: 16) |
| Isl2 | NP_665804.1 | NM_145805.1 | NP_081673.2 | NM_027397.3 |
| | (SEQ ID NO: 7) | (SEQ ID NO: 8) | (SEQ ID NO: 17) | (SEQ ID NO: 18) |
| Klf7 | NP_003700.1 | NM_003709.3 | NP_291041.2 | NM_033563.2 |
| | (SEQ ID NO: 9) | (SEQ ID NO: 10) | (SEQ ID NO: 19) | (SEQ ID NO: 20) |

As used herein, “Asc11” is refers to the Asc11 protein of Genbank accession No: NP_004307.2 (SEQ ID NO: 1) (human), and is encoded by gene NM_004316.3 (SEQ ID NO:2) (human), respectively. The term Asc11 also encompasses species variants, homologues, allelic forms, mutant forms, and equivalents thereof, including conservative substitutions, additions, and deletions therein not adversely affecting the structure of function. Asc11 is referred in the art as aliases; Homo sapiens achaete-scute complex homolog 1 (Drosophila) (ASCL1), ASH1; bHLHa46; HASH1; MASH1.

As used herein, “Myt11” is refers to the Asc11 protein of Genbank accession No: XP_005264742.1 (SEQ ID NO: 3) (human), and is encoded by gene XM_005264685 (SEQ ID NO: 4) (human), respectively. The term Myt11 also encompasses species variants, homologues, allelic forms, mutant forms, and equivalents thereof, including conservative substitutions, additions, and deletions therein not adversely affecting the structure of function. Myt11 is referred in the art as aliases; myelin transcription factor 1-like (MYT1L), KIAA1106, “neural zinc finger transcription factor 1”, NZF1.

As used herein, “Ngn1” is refers to the Asc11 protein of Genbank accession No: NP_006152.2 (SEQ ID NO: 5) (human), and is encoded by gene NM_006161.2 (SEQ ID NO: 6) (human), respectively. The term Ngn1 also encompasses species variants, homologues, allelic forms, mutant forms, and equivalents thereof, including conservative substitutions, additions, and deletions therein not adversely affecting the structure of function. Ngn1 is referred in the art as aliases: Neurogenin-1, Neurog1, neurogenic differentiation 3, “Neurogenic Differentiation Factor 3,” “Class A Basic Helix-Loop-Helix Protein 6,” NeuroD3, and BHLHa6.

As used herein, “Isl2” is refers to the Asc11 protein of Genbank accession No: NP_665804.1 (SEQ ID NO: 7) (human), and is encoded by gene NM_145805.1 (SEQ ID NO:8) (human), respectively. The term Isl2 also encompasses species variants, homologues, allelic forms, mutant forms, and equivalents thereof, including conservative substitutions, additions, and deletions therein not adversely affecting the structure of function. Isl2 is referred in the art as aliases: “ISL LIM Homeobox 2,” “ISL2 Transcription Factor, LIM/Homeodomain (Islet-2),” “Insulin Gene Enhancer Protein ISL-2,” and Islet-2.

As used herein, “Klf7” is refers to the Asc11 protein of Genbank accession No: NP_003700.1 (SEQ ID NO:9) (human), and is encoded by gene NM_003709.3 (SEQ ID NO:10) (human), respectively. The term Klf7 also encompasses species variants, homologues, allelic forms, mutant forms, and equivalents thereof, including conservative substitutions, additions, and deletions therein not adversely affecting the structure of function. Klf7 is referred in the art as aliases: “Kruppel-Like Factor 7,” and “Ubiquitous Kruppel-Like Transcription Factor 7.”

The term “functional fragments” as used herein regarding Asc11, Myt11, Ngn1, Isl2, Klf7, or NeruoD1 polypeptides having amino acid sequences substantially homologous thereto means a polypeptide sequence of at least 5 contiguous amino acids of Asc11, Myt11, Ngn1, Isl2, Klf7 sequences of SEQ ID NO: 1, 3, 5, 7, 9, 11, 13, 15, 17, or 19 having amino acid sequences substantially homologous thereto, wherein the functional fragment polypeptide sequence is about at least 50%, or 60% or 70% or at 80% or 90% or 100% or greater, for example 1.5-fold, 2-fold, 3-fold, 4-fold or greater than 4-fold as effective at direct conversion of a somatic cell, e.g., fibroblast to a iNociceptors as the corresponding wild type Asc11, Myt11, Ngn1, Isl2, or Klf7 polypeptides of SEQ ID NO: 1, 3, 5, 7, 9, 11, 13, 15, 17, or 19, respectively as described herein. The functional fragment polypeptide may have additional functions that can include decreased antigenicity, increased DNA binding (as in transcription factors), or altered RNA binding (as in regulating RNA stability or degradation).

In some embodiments, a somatic cell, e.g., fibroblast can be isolated from a subject, for example as a tissue biopsy, such as, for example, a skin biopsy. In some embodiments, the a somatic cell, e.g., fibroblast are maintained in culture by methods known by one of ordinary skill in the art, and in some embodiments, propagated prior to being directly converted into iNociceptors by the methods as disclosed herein.

Further, a somatic cell, e.g., fibroblast can be from any mammalian species, with non-limiting examples including a murine, bovine, simian, porcine, equine, ovine, or human cell. For clarity and simplicity, the description of the methods herein refers to a mammalian somatic cell, e.g., fibroblast, but it should be understood that all of the methods described herein can be readily applied to other cell types of somatic cells. In one embodiment, the somatic cell, e.g., fibroblast is derived from a human individual, wherein the suitable nociceptor inducing factors are human. In alternative embodiments, the fibroblast is derived from a mouse subject, and wherein the suitable nociceptor inducing factors are mouse. In some embodiments, mouse nociceptor inducing factors can be used to directly convert human fibroblasts to iNociceptors and vice versa, human nociceptor inducing factors can be used for transdifferentiation of mouse fibroblasts into iNociceptors. In some embodiments, any combination of mouse or human nociceptor inducing factors can be used for transdifferentiation of mouse or human fibroblasts into iNociceptors.

In some embodiments, a subject from which a somatic cell, e.g., fibroblast are obtained is a mammalian subject, such a human subject, and in some embodiments, the subject is suffering from a nociceptor pain related disease or disorder. In such embodiments, the a somatic cell, e.g., fibroblast can be transdifferentiated into a iNociceptors ex vivo by the methods as described herein and then administered to the subject from which the cells were harvested in a method to treat the subject for the nociceptive pain related disease or disorder.

In some embodiments, a somatic cell, e.g., fibroblast are located within a subject (in vivo) and are directly converted to become an iNociceptors by the methods as disclosed herein in vivo. In some embodiments, direct conversion of a somatic cell, e.g., a fibroblast to a iNociceptor in vivo can be achieved transducing the fibroblast with a viral vector, such as adenovirus which has the ability to express induced nociceptor factors selected from Asc11, Myt11, Ngn1, and Isl2 and Klf71 in the somatic cell.

In some embodiments, such contacting may be performed by maintaining the somatic cell, e.g., fibroblast in culture medium comprising the agent(s). In some embodiments a somatic cell, e.g., fibroblast can be genetically engineered. In some embodiments, a somatic cell, e.g., fibroblast can be genetically engineered to express induced nociceptor factors selected from Asc11, Myt11, Ngn1, and Isl2, Klf71, or an amino acid sequences substantially homologous thereof, or functional fragments or functional variants thereof.

Where the somatic cell, e.g., fibroblast is maintained under in vitro conditions, conventional tissue culture conditions and methods can be used, and are known to those of skill in the art. Isolation and culture methods for various cells are well within the abilities of one skilled in the art.

Generating iNociceptor by direct conversion of a somatic cell, e.g., fibroblast using the methods of the present invention has a number of advantages. First, the methods of the present invention allow one to generate autologous iNociceptors, which are cells specific to and genetically matched with an individual. The cells are derived from a somatic cell, e.g., fibroblast obtained from the individual. In general, autologous cells are less likely than non-autologous cells to be subject to immunological rejection.

Second, the methods of the present invention allow the artisan to generate iNociceptors without using embryos, oocytes, and/or nuclear transfer technology. Herein, the applicants' results demonstrate that a somatic cell, e.g., fibroblast can be directly converted to become a nociceptor (iNociceptor), without the need to be fully reprogrammed to a pluripotent state, therefore minimizing the risk of differentiation into unwanted cell types or risk of teratomas formation.

Also encompassed in the methods of the present invention is a method of transdifferentiation of a somatic cell, e.g., fibroblast by means other than engineering the cells to express nociceptor inducing factors, i.e., by contacting the a somatic cell, e.g., fibroblast with a nociceptor inducing factors other than a nucleic acid or viral vector capable of being taken up and causing a stable genetic modification to the cells. In particular, the invention encompasses the recognition that extracellular signaling molecules, e.g., molecules that when present extracellularly bind to cell surface receptors and activate intracellular signal transduction cascades, are of use to reprogram somatic cells. The invention further encompasses the recognition that activation of such signaling pathways by means other than the application of extracellular signaling molecules is also of use to directly convert a somatic cell, e.g., fibroblast into a iNociceptors. The present disclosure thus reflects several fundamentally important advances in the area of somatic cell transdifferentiation technology, in particular direct conversion of somatic cells to a subtype of neurons, in particular, nociceptors.

Also encompassed in the methods of the present invention is a method of transdifferentiation of a somatic cell, e.g., fibroblast by means other than engineering the cells to express nociceptor inducing factors, i.e., by contacting the a somatic cell, e.g., fibroblast with a nociceptor inducing factors other than a nucleic acid or viral vector capable of being taken up and causing a stable genetic modification to the cells. In particular, the invention encompasses the recognition that extracellular signaling molecules, e.g., molecules that when present extracellularly bind to cell surface receptors and activate intracellular signal transduction cascades, are of use to reprogram somatic cells. The invention further encompasses the recognition that activation of such signaling pathways by means other than the application of extracellular signaling molecules is also of use to directly convert a somatic cell, e.g., fibroblast into a iNociceptor. The present disclosure thus reflects several fundamentally important advances in the area of somatic cell transdifferentiation technology, in particular direct conversion of somatic cells to a subtype of neurons, in particular, nociceptors.

Another aspect of the present invention relates to methods to produce a population of isolated iNociceptors by increasing the protein expression of five nociceptor inducing factors in a population of a somatic cell, e.g., fibroblast. In some embodiments, a somatic cell, e.g., fibroblast can be treated in any of a variety of ways to cause direct conversion of the fibroblast to an iNociceptor according to the methods of the present invention. For example, in some embodiments, the treatment can comprise contacting the cells with one or more agent(s), herein referred to as a “nociceptor inducing factors” which increases the protein expression of at least the transcription factors selected from Asc11, Myt11, Ngn1, Isl2, Klf7, or increases the protein expression of a functional homologue or a functional fragment of the transcription factors selected from Asc11, Myt11, Ngn1, Isl2, Klf7 polypeptides in the somatic cell, e.g., fibroblast.

In some embodiments, the method comprises converting a somatic cell, e.g., fibroblast by increasing the protein expression of at least three in any combination of the following nociceptor inducing factors selected from Asc11, Myt11, Ngn1, Isl2, Klf7, in the somatic cell, e.g., fibroblast, wherein the expression is for sufficient amount of time, typically transient increase in expression, to allow the conversion of the cell to become a cell which exhibits at least two characteristics of an endogenous nociceptor cell (e.g., expression of at least two nociceptor specific genes selected from the group consisting of TrpA1, TrpM8, P2X7, NaV1.8, Prph and CGRP). The increase in expression of the transcription factors can be done all at the same time (e.g. concurrently), or alternatively, subsequently in any order.

In some embodiments, increasing the protein expression can be by any means known by one of ordinary art, for example can include introduction of nucleic acid, or nucleic acid analogue encoding one or more of the nociceptor inducing factors, or contacting the somatic cell, e.g., fibroblast with an agent which converts the somatic cell, e.g., fibroblast to a cell with a nociceptor phenotype. In some embodiments, a nucleic acid analogue is a locked nucleic acid (LNA), or a modified synthetic RNA (modRNA) encoding one or more of the nociceptor inducing factors.

In some embodiments, a nociceptor inducing factor is a vector comprising a nucleotide sequence encoding the polypeptide one or more of Asc11 (SEQ ID NO:1), Myt11 (SEQ ID NO:3), Ngn1 (SEQ ID NO:5), Isl2 (SEQ ID NO:7), Klf7 (SEQ ID NO:9), or encoding a polypeptide substantially homologous to SEQ ID NO:1, 3, 5, 7, or 9 or a functional variant or functional fragment of polypeptides of sequences SEQ ID NO: 1, 3, 5, 7, or 9. In such embodiments, the nucleotide sequence can comprise any nucleic acid sequence selected from SEQ ID NO: 1, 3, 5, 7, or 9 respectively, or a fragment or variant thereof.

In some embodiments, the vector is a viral vector. In some embodiments, the viral vector is a non-integrating viral vector. While retroviral vectors incorporate into the host cell genome and can potentially disrupt normal gene function, non-integrating vectors have the advantage of controlling expression of a gene product by extra-chromosomal transcription. It follows that since non-integrating vectors do not become part of the host genome, non-integrating vectors tend to express a nucleic acid transiently in a cell population. This is due in part to the fact that the non-integrating vectors as used herein are rendered replication deficient. Thus, non-integrating vectors have several advantages over retroviral vectors including but not limited to: (1) no disruption of the host genome, and (2) transient expression, and (3) no remaining viral integration products.

In other embodiments, the methods or the present invention encompass non-viral means to increase the expression of nociceptor inducing factors in a somatic cell, e.g., fibroblast for the purposes for converting to an iNociceptors as disclosed herein. For example, in one embodiment, naked DNA technology can be used, for example nucleic acid encoding the polypeptides of least three transcription factors selected from Asc11, Myt11, Ngn1, Isl2, Klf7, (encoded by SEQ ID NO: 2, 4, 6, 8 and 10 respectively) can be introduced into a somatic cell, e.g., fibroblast for the purposes of converting the cell to an iNociceptors.

In alternative embodiments, one can contact the somatic cell, e.g., fibroblast with a small molecule or combination of small molecules (e.g. chemical complementation) to increase the expression of at least two transcription factors in the somatic cell, e.g., fibroblast.

Thus, in some embodiments, the contacting step will typically be for at least twenty-four hours. By “at least twenty-four hours,” is meant twenty-four hours or greater. In some embodiments, fibroblast cells can be contacted with nociceptor inducing factor (e.g. small molecule, polypeptide, nucleic acid, nucleic acid analogues, etc.) for about 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 hours up to 3, 4, 5, 6, 7, or more days or any particular intervening time in hours or minutes within the above range. Preferably, somatic cells, e.g., fibroblasts can be contacted with a nociceptor inducing agent for seven days.

To confirm the transdifferentiation of a somatic cell, e.g., fibroblast to an iNociceptors, isolated clones can be tested for the expression of a marker of nociceptors. Such expression identifies the cells as a nociceptor cell. Markers for nociceptors can be selected from the non-limiting group including TrpA1, TrpV1, P2X3, Nav1.7, NaV1.8, Prph and CGRP, where expression is by a statistically significant amount as compared to the somatic cell, e.g., fibroblast from which the iNociceptor was converted from.

Methods for detecting the expression of such markers are well known in the art, and include, for example, RT-PCR and immunological methods that detect the presence of the encoded polypeptides, such as ELISA.

Another aspect of the present invention relates to the isolation of a population of iNociceptors from a heterogeneous population of cells, such a comprising a mixed population of iNociceptors and somatic cells from which the iNociceptors were derived. A population of iNociceptor produced by any of the above-described processes can be enriched, isolated and/or purified by using an affinity tag that is specific for such cells. Examples of affinity tags specific for iNociceptor are antibodies, ligands or other binding agents that are specific to a marker molecule, such as a polypeptide, that is present on the cell surface of iNociceptor but which is not substantially present on other cell types (i.e. on the a somatic cell, e.g., fibroblast) that would be found in the heterogeneous population of cells produced by the methods described herein. In some processes, an antibody which binds to a cell surface antigen on human iNociceptor is used as an affinity tag for the enrichment, isolation or purification of iNociceptor produced by in vitro methods, such as the methods described herein. Such antibodies are known and commercially available.

In one embodiment of the above methods, the population of iNociceptors as disclosed herein are human cells.

The skilled artisan will readily appreciate that the processes for making and using antibodies for the enrichment, isolation and/or purification of iNociceptor are also readily adaptable for the enrichment, isolation and/or purification of iNociceptor. For example, analyzing and sorting for iNociceptors using a fluorescence activated cell sorter (FACS). Antibody-bound, fluorescent cells are collected separately from non-bound, non-fluorescent, thereby resulting in the isolation of such cell types.

In preferred embodiments of the processes described herein, the isolated cell composition comprising iNociceptor can be further purified by using an alternate affinity-based method or by additional rounds of sorting using the same or different markers that are specific for iNociceptor.

In preferred processes, iNociceptors are enriched, isolated and/or purified from other non-iNociceptor s (i.e. from a somatic cell, e.g., fibroblast which have not been reprogrammed to become iNociceptors) after the cell population is induced to reprogram towards a nociceptor lineage using the methods and compositions as disclosed herein.

In addition to the procedures just described, iNociceptors may also be isolated by other techniques for cell isolation. Additionally, iNociceptors may also be enriched or isolated by methods of serial subculture in growth conditions which promote the selective survival or selective expansion of iNociceptors.

Using the methods described herein, enriched, isolated and/or purified populations of iNociceptors cells can be produced in vitro from a somatic cell, e.g., fibroblast, which has undergone sufficient transdifferentiation to produce at least some iNociceptors. In a preferred method, a population of somatic cells, e.g., fibroblasts can be trandifferentiated primarily into a population of iNociceptors, where only a portion of the somatic cell population, e.g., about 5-10% has converted to iNociceptors. Some preferred enrichment, isolation and/or purification methods relate to the in vitro production of iNociceptors from human a somatic cell, e.g., fibroblast.

The use of an isolated population of iNociceptors as disclosed herein provides advantages over existing methods because the iNociceptors can be reprogrammed from a somatic cell, e.g., fibroblast obtained or harvested from the subject administered an isolated population of iNociceptors. In another embodiment, an isolated population of iNociceptors can be used as models for studying properties of nociceptors, or pathways of development of a somatic cell, e.g., fibroblast into a nociceptor cell.

Some embodiments of the present invention relate to cell compositions, such as cell cultures or cell populations, comprising iNociceptors, wherein the iNociceptors are nociceptors which have been derived from cells e.g. human a somatic cell, e.g., fibroblast, which express or exhibit one or more characteristics of an endogenous nociceptors. In accordance with certain embodiments, the iNociceptors are mammalian cells, and in a preferred embodiment, such cells are human iNociceptors.

Other embodiments of the present invention relate to compositions, such as cell cultures or cell populations, comprising iNociceptors. In such embodiments, somatic cells, e.g., fibroblasts comprise less than about 90%, less than about 85%, less than about 80%, less than about 75%, less than about 70%, less than about 65%, less than about 60%, less than about 55%, less than about 50%, less than about 45%, less than about 40%, less than about 35%, less than about 30%, less than about 25%, less than about 20%, less than about 15%, less than about 12%, less than about 10%, less than about 8%, less than about 6%, less than about 5%, less than about 4%, less than about 3%, less than about 2% or less than about 1% of the total cells in the cell population.

Certain other embodiments of the present invention relate to compositions, such as cell cultures or cell populations, comprising iNociceptors. In some embodiments, a somatic cell, e.g., fibroblast from which the iNociceptors are derived comprise less than about 25%, less than about 20%, less than about 15%, less than about 10%, less than about 5%, less than about 4%, less than about 3%, less than about 2% or less than about 1% of the total cells in the culture. In certain embodiments, iNociceptors comprise less than about 25%, less than about 20%, less than about 15%, less than about 10%, less than about 5%, less than about 4%, less than about 3%, less than about 2% or less than about 1% of the total cells in the culture.

Additional embodiments of the present invention relate to compositions, such as cell cultures or cell populations, produced by the processes described herein and which comprise iNociceptors as the majority cell type. In some embodiments, the processes described herein produce cell cultures and/or cell populations comprising at least about 99%, at least about 98%, at least about 97%, at least about 96%, at least about 95%, at least about 94%, at least about 93%, at least about 92%, at least about 91%, at least about 90%, at least about 89%, at least about 88%, at least about 87%, at least about 86%, at least about 85%, at least about 84%, at least about 83%, at least about 82%, at least about 81%, at least about 80%, at least about 79%, at least about 78%, at least about 77%, at least about 76%, at least about 75%, at least about 74%, at least about 73%, at least about 72%, at least about 71%, at least about 70%, at least about 69%, at least about 68%, at least about 67%, at least about 66%, at least about 65%, at least about 64%, at least about 63%, at least about 62%, at least about 61%, at least about 60%, at least about 59%, at least about 58%, at least about 57%, at least about 56%, at least about 55%, at least about 54%, at least about 53%, at least about 52%, at least about 51% or at least about 50% iNociceptors. In preferred embodiments, the cells of the cell cultures or cell populations comprise human cells. In other embodiments, the processes described herein produce cell cultures or cell populations comprising at least about 50%, at least about 45%, at least about 40%, at least about 35%, at least about 30%, at least about 25%, at least about 24%, at least about 23%, at least about 22%, at least about 21%, at least about 20%, at least about 19%, at least about 18%, at least about 17%, at least about 16%, at least about 15%, at least about 14%, at least about 13%, at least about 12%, at least about IT %, at least about 10%, at least about 9%, at least about 8%, at least about 7%, at least about 6%, at least about 5%, at least about 4%, at least about 3%, at least about 2% or at least about 1% iNociceptors. In preferred embodiments, the cells of the cell cultures or cell populations comprise human cells. In some embodiments, the percentage of iNociceptors in the cell cultures or populations is calculated without regard to the feeder cells remaining in the culture.

Still other embodiments of the present invention relate to compositions, such as cell cultures or cell populations, comprising mixtures of iNociceptors and a somatic cell, e.g., fibroblast. For example, cell cultures or cell populations comprising at least about 5 iNociceptors for about every 95 somatic cell, e.g., fibroblast can be produced. In other embodiments, cell cultures or cell populations comprising at least about 95 iNociceptors for about every 5 somatic cell, e.g., fibroblast can be produced. Additionally, cell cultures or cell populations comprising other ratios of iNociceptors to somatic cell, e.g., fibroblast are contemplated. For example, compositions comprising at least about 1 iNociceptors for about every 1,000,000, or at least 100,000 cells, or at least 10,000 cells, or at least 1000 cells or 500, or at least 250 or at least 100 or at least 10 somatic cell, e.g., fibroblast. Further embodiments of the present invention relate to compositions, such as cell cultures or cell populations, comprising human cells, including human iNociceptors.

In preferred embodiments of the present invention, cell cultures and/or cell populations of iNociceptors comprise human iNociceptors that are non-recombinant cells. In such embodiments, the cell cultures and/or cell populations are devoid of or substantially free of recombinant human iNociceptors.

Using the processes described herein, compositions comprising iNociceptors are substantially free of other cell types can be produced. In some embodiments of the present invention, the iNociceptors populations or cell cultures produced by the methods described herein are substantially free of cells that significantly express the fibroblast markers, or non-nociceptor markers.

Another aspect of the present invention further provides a method of treating a subject with a nociceptive pain related disease or disorder, or treating a subject at risk of developing a nociceptive pain related disease or disorder, comprising administering to the subject a composition comprising a population of iNociceptors. In some embodiments the nociceptive pain related disease or disorder is pain accompanying a disease selected from the group consisting of rheumatoid arthritis, rheumatoid spondylitis, osteoarthritis, spondylosis deformans, gouty arthritis, juvenile arthritis, scapulohumeral periarthritis, fibromyalgia, and cervical syndrome; lumbago; lumbago accompanying spondylosis deformans; menalgia; pain and tumentia after inflammation, surgery or injury; pain after odontectomy; and cancer pain.

In some embodiments, the present invention also provides a method of treating a nociceptive pain related disease or disorder in a subject, comprising administering a substantially pure population of iNociceptors to the subject.

In some embodiments, an iNociceptor population as disclosed herein may serve for testing and high throughput screening of molecules for the treatment of nociceptive pain related disease or disorder.

The subject of the invention can include individual humans, domesticated animals, livestock (e.g., cattle, horses, pigs, etc.), and pets (like cats and dogs).

Accordingly, the methods for treatment as described herein can be combined with other methods of treating a nociceptive pain related disease or disorder which are known by a skilled physician in the art of neurological treatment of nociceptive pain.

The cells and components such as one or more Nociceptor inducing factors can be provided in a kit. The kit includes (a) the cells and components described herein, e.g., a composition(s) that includes a cell and component(s) described herein, and, optionally (b) informational material. The informational material can be descriptive, instructional, marketing or other material that relates to the methods described herein and/or the use of a compound(s) described herein for the methods described herein.

The informational material of the kits is not limited in its form. In one embodiment, the informational material can include information about production of a cell, the nature of the components such as the transcription factor, concentration of components, date of expiration, batch or production site information, and so forth. In one embodiment, the informational material relates to methods for administering the cells or other components.

In one embodiment, the informational material can include instructions to administer a compound(s) component such as a transcription factor described herein in a suitable manner to perform the methods described herein, e.g., in a suitable dose, dosage form, or mode of administration (e.g., a dose, dosage form, or mode of administration described herein) (e.g., to a cell in vitro or a cell in vivo). In another embodiment, the informational material can include instructions to administer a component(s) described herein to a suitable subject, e.g., a human, e.g., a human having or at risk for a disorder described herein or to a cell in vitro.

The informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as Braille, computer readable material, video recording, or audio recording. In another embodiment, the informational material of the kit is contact information, e.g., a physical address, email address, website, or telephone number, where a user of the kit can obtain substantive information about a compound described herein and/or its use in the methods described herein. Of course, the informational material can also be provided in any combination of formats.

In addition to a compound(s) described herein, the composition of the kit can include other ingredients, such as a solvent or buffer, a stabilizer, a preservative, and/or an additional agent, e.g., for reprogramming a somatic cell, e.g., fibroblast, such as a somatic cell (e.g., in vitro or in vivo) or for treating a condition or disorder described herein. Alternatively, the other ingredients can be included in the kit, but in different compositions or containers than a component described herein. In such embodiments, the kit can include instructions for admixing a component(s) described herein and the other ingredients, or for using a component(s) described herein together with the other ingredients, e.g., instructions on combining the two agents prior to administration.

The kit can include one or more containers for the composition containing a component(s) described herein. In some embodiments, the kit contains separate containers (e.g., two separate containers for the two agents), dividers or compartments for the component(s) and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of a compound described herein. For example, the kit includes a plurality of syringes, ampules, foil packets, or blister packs, each containing a single unit dose of a component described herein. The containers of the kits can be air tight, waterproof (e.g., impermeable to changes in moisture or evaporation), and/or light-tight.

The methods described herein include the manufacture and use of pharmaceutical compositions, which include iNociceptors produced by a method described herein as active ingredients. Pharmaceutical compositions comprising effective amounts of a population of iNociceptors are also contemplated by the present invention. These compositions comprise an effective number iNociceptors, optionally, in combination with a pharmaceutically acceptable carrier, additive or excipient. In certain aspects of the present invention, a population of iNociceptors can be administered to the subject in need of a transplant in sterile saline. In other aspects of the present invention, a population of iNociceptors can be administered in Hanks Balanced Salt Solution (HBSS) or Isolyte S, pH 7.4. Other approaches may also be used, including the use of serum free cellular media. In one embodiment, a population of iNociceptors can be administered in plasma or fetal bovine serum, and DMSO. Systemic administration of a population of iNociceptors to the subject may be preferred in certain indications, whereas direct administration at the site of or in proximity to the diseased and/or damaged tissue may be preferred in other indications.

In some embodiments, a population of iNociceptors can optionally be packaged in a suitable container with written instructions for a desired purpose, such as the reconstitution or thawing (if frozen) of a population of iNociceptors prior to administration to a subject.

In one embodiment, an isolated population of iNociceptors as disclosed herein can be administered with a differentiation agent. In one embodiment, iNociceptors can be combined with the differentiation agent to administration into the subject. In another embodiment, the cells are administered separately to the subject from the differentiation agent. Optionally, if the cells are administered separately from the differentiation agent, there is a temporal separation in the administration of the iNociceptors and the differentiation agent. The temporal separation may range from about less than a minute in time, to about hours or days in time. The determination of the optimal timing and order of administration is readily and routinely determined by one of ordinary skill in the art.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration.

Pharmaceutical compositions are typically formulated to be compatible with its intended route of administration. Examples of routes of administration include parenteral, e.g., intravenous, intradermal, subcutaneous, oral (e.g., inhalation), transdermal (topical), transmucosal, and rectal administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, N.Y.). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

The pharmaceutical compositions can also be prepared in the form of suppositories (e.g., with conventional suppository bases such as cocoa butter and other glycerides) or retention enemas for rectal delivery.

Therapeutic compounds that are or include nucleic acids can be administered by any method suitable for administration of nucleic acid agents, such as a DNA vaccine. These methods include gene guns, bio injectors, and skin patches as well as needle-free methods such as the micro-particle DNA vaccine technology disclosed in U.S. Pat. No. 6,194,389, and the mammalian transdermal needle-free vaccination with powder-form vaccine as disclosed in U.S. Pat. No. 6,168,587. Additionally, intranasal delivery is possible, as described in, inter alia, Hamajima et al., Clin. Immunol. Immunopathol., 88(2), 205-10 (1998). Liposomes (e.g., as described in U.S. Pat. No. 6,472,375) and microencapsulation can also be used. Biodegradable targetable microparticle delivery systems can also be used (e.g., as described in U.S. Pat. No. 6,471,996).

In one embodiment, the iNociceptors are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration.

In a further aspect, the disclosure provides a method of evaluating a drug for protecting against neuropathic pain by blocking nociceptor sensitization in response treatment (e.g., treatment with inflammatory mediators such as Prostaglandin E2 (PGE2) or chemotherapeutic agents such as oxaliplatin), comprising, a) providing: i) a nociceptor, and ii) a test compound b) contacting said nociceptor with said test compound and measuring activation or inhibition of nociceptor function, nociceptor sensitization, nocicepotor survival or growth. In one embodiment, said nociceptor is derived from a somatic cell (e.g., a fibroblast).

In yet another aspect, the disclosure provides methods for determining a patient's susceptibility to cancer chemotherapy induced pain and peripheral neuropathy by measuring nociceptor sensitization in response to a chemotherapeutic agent. The methods are particularly useful for optimizing treatment outcomes for patients undergoing chemotherapy. The methods comprising the steps of contacting an induced nociceptor cell obtained from a patient with a chemotherapeutic agent, contacting the induced nociceptor cell with nociceptor sensitizing agent (e.g., capsaicin or mustard oil) following exposure to the chemotherapeutic agent, measuring a level of nociceptor activation following exposure to the nociceptor sensitizing agent, comparing the measured level of nociceptor activation following exposure to the chemotherapeutic agent and the nociceptor sensitizing agent with a reference level of nociceptor activation obtained from a nociceptor cell following exposure to the control agent and the nociceptor sensitizing agent, identifying the measured level of nociceptor activation as above the reference level of nociceptor activation, which is indicative of an increase susceptibility to cancer chemotherapy induced pain and peripheral neuropathy, or identifying the measured level of nociceptor activation as the same or below the reference level of nociceptor activation, which is indicative of an increase susceptibility to cancer chemotherapy induced pain and peripheral neuropathy; and modifying the patient's clinical treatment program based on the patient's susceptibility to cancer chemotherapy induced pain and peripheral neuropathy.

Modifying the patient's clinical treatment program may include, for example, administering an to the patient a different chemotherapeutic agent, altering the combination of treatments, reducing the dose or exposure time to a the chemotherapeutic agent, or administering a neuroprotective therapy to patient's determined to be susceptibility to cancer chemotherapy induced pain and peripheral neuropathy

In a further aspect, the methods disclosed herein further comprise modifying the subject's clinical record to identify the subject as being susceptible to cancer chemotherapy induced pain and peripheral neuropathy. The clinical record maybe be stored in any suitable data storage medium (e.g., a computer readable medium).

The present methods can also be used for selecting a treatment and/or determining a treatment plan for a patient, based on the patient's susceptibility to cancer chemotherapy induced pain and peripheral neuropathy. In some embodiments, using the method disclosed herein, a health care provider (e.g., a physician) identifies (i.e., diagnoses) a patient as being susceptibility to cancer chemotherapy induced pain and peripheral neuropathy and, based on this identification the health care provider determines an adequate treatment management plan for the subject. By way of this diagnosis the health care provider determines an adequate treatment or treatment plan for the subject. In some embodiments, the methods further include administering the treatment to the subject.

Chemotherapeutic agent contemplated for use in the methods disclosed herein include, for example, 5-fluorouracil, hydroxyurea, anthracyclins, taxol, taxotere, tamoxifen, anti-estrogens, interferons, bendamustine, busulfan, carmustine, chlorambucil, cyclophosphamide, dacarbazine, ifosfamide, melphalan, procarbazine, streptozocin, temozolomide, asparaginase, capecitabine, cytarabine, fludarabine, cemcitabine, methotrexate, pemetrexed, raltitrexed, actinomycin D/dactinomycin, bleomycin, daunorubicin, doxorubicin, doxorubicin (pegylated liposomal), epirubicin, idarubicin, mitomycin, mitoxantrone, etoposide, docetaxel, irinotecan, paclitaxel, topotecan, vinblastine, vincristine, vinorelbine, carboplatin, cisplatin and oxaliplatin.

### EXAMPLES

The invention is further described in the following examples, which do not limit the scope of the invention described in the claims.

The examples presented herein relate to the methods and compositions for induced nociceptor (i-nociceptors) from somatic cells, e.g., fibroblasts by increasing the expression of at least five nociceptor-inducing factors selected from Asc11, Myt11, Isl2, Ngn1, and Klf7, for example by using nucleic acid sequences to encoding the proteins Asc11, Myt11, Isl2, Ngn1, Klf7 of SEQ ID NO: 1, 3, 5, 7 or 9 (human), or proteins Asc11, Myt11, Isl2, Ngn1, Klf7 of SEQ ID NO: 11, 13, 15, 17, or 19 (murine) or functional fragments thereof. The following examples are not intended to limit the scope of the claims to the invention, but are rather intended to be exemplary of certain embodiments. Any variations in the exemplified methods which occur to the skilled artisan are intended to fall within the scope of the present invention.

Epigenetic reprogramming of somatic cells from one cell fate to another can generate specific cell subtypes for disease modeling. To use this technique to derive noxious stimulus-detecting (nociceptor) neurons, the inventors chose candidate transcription factors based both on the literature and the transcriptome of sorted adult mouse nociceptors. The inventors optimized the selection by empiric combination and drop out studies to obtain five key transcription factors that drive cells to the TrpV1 lineage. These factors reprogrammed mouse and human fibroblasts to neurons that recapitulate many specific physiological features of nociceptor neurons, including the capacity to detect noxious stimuli via functional TrpV1, TrpA1 and P2X3 receptors. The derived nociceptor neurons mimic, moreover, a disease-relevant process, sensitization to the inflammatory mediator prostaglandin E2, thus modeling the inherent cellular biology necessary for generating inflammatory pain hypersensitivity. Using fibroblasts from a patient with familial dysautonomia (hereditary sensory and autonomic neuropathy type III), the inventors show that the technique can aid investigation of human disease phenotypes in vitro.

### Materials and Methods

### Fibroblasts.

TrpV1::tdTomato-transgenic mice were obtained by crossing TrpV1-Cre^{+/+} mice with tdTomato^{+/+} reporter mice (both from Jackson Laboratories). Mouse embryonic fibroblasts (MEFs) were harvested from TrpV1::tdTomato embryos at E12.5, passaged once and frozen at −120° C. Human fibroblasts were obtained from a healthy control subject (ATCC CRL-2522) and from a subject with familial dysautonomia (Coriell Institute GM04663, 2 year old Caucasian female). The use of human lines was approved under the Boston's Children's Hospital Institutional Review Board.

### Viruses and Transductions.

Complimentary DNAs (cDNAs) for the 9 candidate factors (obtained from the Dana Farber/Harvard Cancer Center DNA Resource Core except Ngn1, Tlx3 and Runx1, which were obtained from Q. Ma) were each cloned into the pMXs retroviral expression vector modified to contain a woodchuck response element (“WRE”) (Ichida & Eggan) using Gateway technology (Invitrogen). 293T cells were co-transfected with individual viruses and pHDMG and pIKLMV packaging plasmids using Lipofectamine 2000 (Life Technologies). Media was changed to new DMEM (GIBCO), 20% FBS (Invitrogen), 50 U/mL Penicillin/Streptomycin (CellGro) after 16 hours. At that time, fibroblasts were thawed and plated on 24-well plates (25K cells/well), 6-well plates (150K cells/well), 35 mm dishes (150K cells/well), or p515A multi-electrode array (MEA) probes (Alpha Med Scientific) (12K cells/MEA) that were previously coated with poly-D-lysine (Sigma) overnight at room temperature, followed by three washes with dH₂O, gelatin coating (Cell Signaling) for 1 hour at 37° C., and laminin coating (Sigma) for 1-2 hours at 37° C. Viruses were harvested 24 hours later, concentrated approximately 5 fold using amicon ultra centrifugal filter units (Millipore) and applied to fibroblasts with 5 μg/ml polybrene (Day 0, transduction). Cortical mouse glia obtained from P0-P2 C57Bl6 mice were added on Day 2 for all but the calcium imaging experiments. Media was switched on Day 4 to N3 media (DMEM/F-12 (GIBCO), N2 and B27 supplements (Life Technologies), glutaMAX (Invitrogen), pen/strep, FGF (20 ng/mL, Millipore) with 5% FBS, along with the growth factors BDNF, CNTF, GDNF at 10 ng/ml each. The TGFβ-Inhibitor RepSox (7.5 μM; Millipore), which has been shown to improve survival of different neuronal types over long-term culture (Ichida and Eggan, unpublished), was added for calcium imaging and human transductions. Media was changed every two days, and on Day 10, NGF was also added to the media at (50 ng/mL).

### Immunohistochemistry.

Cells were grown in 24-well plates (Falcon) coated sequentially with gelatin, poly-D-lysine and laminin. The cells were fixed with 4% paraformaldehyde (PFA), washed three times with 1×PBS, incubated in blocking buffer (1% Blocking Reagent (Roche), 0.5% BSA, 0.1% TritonX-100) for one hour at room temperature and stained with primary antibodies overnight at 4° C. in blocking buffer. The next day the cells were washed three times with 1×PBS, stained with secondary antibodies for one hour at room temperature and washed three times with 1×PBS before imaging, which was performed using the microscope setup described below.

Primary antibodies included: mouse anti-β tubulin III (Sigma T8660, 1:1000, validated51), rabbit anti-peripherin (Millipore AB1530, 1:800, validated52), rabbit anti-TrpV1 (Alomone Labs ACC-030, 1:200, validated53), rabbit anti-CGRP (Calbiochem/Millipore PC205L, 1:300, validated54), chicken anti-neurofilament, heavy chain (Millipore AB5539, 1:1000, validated55), mouse anti-Nestin (Abcam ab6142, 1:500, validated56), mouse anti-smooth muscle actin (Sigma A5228, 1:300, validated57), goat anti-Sox1 (Santa Cruz #SC17317, 1:50, validated58), mouse anti-Ki67 (Sigma P6834, 1:500, validated59). Secondary antibodies included: goat anti-chicken AlexaFluor 568 (Life Technologies A11041), goat anti-chicken AlexaFluor 488 (Life Technologies A11039), goat anti-mouse AlexaFluor 488 (Life Technologies A11029), goat anti-mouse AlexaFluor 568 (Life Technologies A11031), goat anti-rabbit AlexaFluor 488 (Life Technologies A11008), goat anti-rabbit AlexaFluor 568 (Life Technologies A11011), donkey anti-goat AlexaFluor 488 (Life Technologies A11055).

### Primary DRG Culture.

DRGs were dissected from adult TrpV1-Cre::tdTomato mice (12-13 weeks) into Hank's balanced salt solution (HBSS) (Life Technologies). DRG were dissociated in 1 mg ml⁻¹ collagenase A plus 2.4 U ml⁻¹ dispase II (enzymes, Roche Applied Sciences) in HEPES-buffered saline (Sigma) for 90 min at 37° C. and then triturated down to single cell level using glass Pasteur pipettes of decreasing size. DRGs were the centrifuged over a 10% BSA gradient and plated on laminin-coated cell culture dishes (Sigma). DRGs were cultured 24 hours in B27-supplemented neurobasal-A medium plus 50 ng/ml nerve growth factor (Invitrogen), 2 ng/ml glial cell derived neurotrophic factor (Sigma), 10 uM arabinocytidine (Sigma) and penicillin/streptomycin (Life Technologies).

Quantitative PCR (qPCR).

To compare expression levels of select genes in TrpV1-tdTomato-positive induced nociceptors, TrpV1-tdTomato-positive primary DRGs and TrpV1-tdTomato MEFs, RNA was harvested with Trizol (Life Technologies). Reverse transcription was completed with a SuperScript VILO cDNA synthesis kit (Life Technologies). Quantitative PCR was completed using mouse-specific TaqMan Gene Expression Assays (Life Technologies) and the TaqMan Gene Expression Master Mix (Life Technologies).

### Single Cell RT-PCR.

Single human induced nociceptors were picked using individual patch pipettes and placed into Single Transcript Amplification (RT-STA) mixture from the CellsDirect One-Step qRT-PCR Kit (Life Technologies) using primers for normally and aberrantly spliced IKBKAP⁴⁰ and GAPDH. RT-STA reaction products were used for PCR using the same IKBKAP and GAPDH primers and resulting products were run on 1% agarose gels.

### Obtaining Fibroblast-Derived Nociceptors, Adult DRGs, and MEFs for FACS.

Induced nociceptors were lifted using Papain (Worthington) with DNase (Worthington) in DMEM/F12 with Pen/Strep. Induced and primary nociceptors were filtered through a 70 μM cell strainer and re-suspended in saline for FACS. Immediately prior to FACS, Dapi was added (20 ng/mL, Sigma) to the cell suspension to exclude dead cells. Cells were FACs sorted directly into Trizol and stored at −80° C. overnight prior to RNA extraction. Separate MEFs were collected directly into Trizol for RNA extraction and comparison.

### Calcium Imaging.

Cells were loaded with Fura2-AM (10 ug/mL, Molecular Probes) by incubating at room temperature for one hour and then de-stained for 15 minutes in sodium chloride-based saline. For primary DRG from adult TrpV1-Cre^{+/-}::tdTomato^{+/-} mice, cells were imaged after 24 hours in culture using an identical protocol except that cells were loaded with Fura-2AM (2 ug/mL) for 45 minutes and then de-stained for 15 minutes. Cells were imaged using a Nikon Eclipse Ti microscope with a Xenon lamp, Andor DL-604M camera and standard 340 nM and 380 nM filters controlled by a Lud1 Mac6000 shutter using Nikon Elements software. Exposure times were 300-600 ms and images were taken every three seconds. One minute of baseline imaging was recorded prior to the addition of the agonists. Menthol (250 uM) was applied at one minute, followed by Mustard oil (100 uM) at two minutes and Capsaicin (1 uM) at three minutes and finally KCl (40 mM) at four minutes. For Trp channel experiments, each agonist was applied for 20 seconds and then washed out with external solution. In the sensitization experiments Capsaicin (300 nM) was applied for 20 seconds after two minutes of recording, followed immediately by PGE2 (1 μM) for two minutes, a conditioned capsaicin (300 nM) application for 20 seconds and KCl (40 mM) after 4.5 minutes. Analysis of tdTomato positive cells was performed using custom Matlab (Mathworks) software to include cells that responded to KCl (1.5×baseline), had a stable baseline and response to agonist with an amplitude of at least 10% of baseline, with subsequent agonist responses required to be both at least 10% of initial baseline and 10% above a second baseline value obtained during the immediately preceding wash period. CGRP ELISA. Induced nociceptors, BAM-derived neurons and primary DRGs were exposed to KCl (20 mM, 40 mM, 60 mM, or 80 mM), capsaicin (0.1 μM), or vehicle for 10 minutes at 37° C. The supernatants were collected and analyzed using the Rat CGRP Enzyme Immunoassay Kit (Bertin Pharma/Cayman Chemical, #589001). Plates were read at 405 nm for 0.1 s on a Wallac Victor2 1420 Multilabel Counter (Perkin Elmer), and data were analyzed using the Wallac 1420 Workstation. Induced nociceptors, BAM-derived neurons and primary DRGs were exposed to KCl (20 mM, 40 mM, 60 mM, or 80 mM), capsaicin (0.1 μM), or vehicle for 10 minutes at 37° C. The supernatants were collected and analyzed using the Rat CGRP Enzyme Immunoassay Kit (Bertin Pharma/Cayman Chemical, #589001). Plates were read at 405 nm for 0.1 s on a Wallac Victor2 1420 Multilabel Counter (Perkin Elmer), and data were analyzed using the Wallac 1420 Workstation.

### Multi-Electrode Array (MEA) Recording.

TrpV1-Cre^{+/-}::tdTomato^{+/-} mouse embryonic fibroblasts (MEFs) were plated on poly-D-lysine/laminin coated p515A probes (Alpha Med Scientific)) at typical densities of 12,000 per probe, transduced with retroviruses and cultured for four weeks. Recordings from 64 extracellular electrodes were made using a Med64 (Alpha Med Scientific) MEA recording amplifier with a head stage that maintained a temperature of 37° C. Data were sampled at 20 kHz, digitized, and analyzed using Mobius software (Alpha Med Scientific) with a 2 kHz 9-pole Bessel low pass filter using a sodium-based extracellular solution: 135 mM NaCl, 5 mM KCl, 2 mM CaCl₂, 1 mM MgCl₂, 10 mM glucose, 10 mM HEPES 10, pH 7.4. The probes were recorded for one minute before the application of the agonists to obtain a baseline and two minutes after the application of capsaicin (1 μM final concentration) or mustard oil (100 μM final concentrations), which were applied at a 10×concentration to the edge of the well (far from the electrodes). The cells were then recorded from for an additional 2 minutes. Three replicates for each agonist, capsaicin and mustard oil, were completed from two separate transductions. The cells tended to migrate away from the electrodes over the course of incubation, which led to increased variability in the final density of neurons on the electrodes. Probes were imaged before recording to ensure sufficient cell density.

### Patch Electrophysiology.

Whole-cell patch recordings were performed on induced tdTomato-positive nociceptors, derived from TrpV1-Cre^{+/-}::tdTomato^{+/-} MEFs, which had been plated at 150,000 cells per 3.5 cm dish. Electrophysiological recordings were performed at four-five weeks post-transduction and assessed responses to capsaicin and α, β-methylene ATP (30 μM, Sigma), total and TTX (300 nM, Sigma)-resistant currents and action potentials and HCN depolarizating current sags. Whole-cell current-clamp and voltage-clamp recordings were performed using a Multiclamp 700B (Molecular Devices) at room temperature (21-23° C.). Data were sampled at 20 kHz and digitized with a Digidata 1440A A/D interface and recorded using pCLAMP 10 software (Molecular Devices). Data were low-pass filtered at 2 kHz. Patch pipettes were pulled from borosilicate glass capillaries on a Sutter Instruments P-97 puller and had resistances of 2-4 MΩ. The pipette capacitance was reduced by wrapping the shank with Parafilm and compensated for using the amplifier circuitry. Series resistance was 5-10 MΩ and compensated by at least 80%.

For voltage-clamp recordings, voltages were elicited by 200-ms depolarizing steps from a holding potential of −80 mV to test potentials ranging from −100 mV to 60 mV in 10 mV increments. Responses to capsaicin (1 uM) and α, β-methylene ATP (30 uM) were measured in voltage clamp at a holding potential of −80 mV. Electrode drift was measured at the end of each recording and was typically 1-2 mV. The potassium-based intracellular solution contained 150 mM KCl, 2 mM MgCl₂, 10 mM HEPES, 4 mM MgATP, 0.3 mM NaGTP, 10 mM Na₂PhosCr, 1 mM EGTA, pH 7.4. For isolation of voltage-gated sodium currents internal KCl was replaced by CsCl to block potassium currents and 100 μM CdCl₂ was used to block calcium currents. For isolation of TTX resistant sodium channels 300 nM TTX were used to block TTX sensitive voltage-gated sodium channels. HCN currents were measured by sequential hyperpolarizing steps in current clamp with an increment of −10 pA steps.

### Quantification of Cell Number, Axon Length and Axonal Branching:

Cells were fixed six weeks following fibroblast transduction with 4% PFA and stained with Peripherin and β-tubulin III, as described above. Induced cell numbers were counted manually and blind to cell line identity for the number of peripherin-positive and β-tubulin III-positive, neuronal-looking cells. Images were taken of 60 neurons from the healthy control fibroblasts and 60 neurons from the fibroblasts from the neuropathy patient. The images were taken from cells derived in three separate experiments by a blinded imager. A blinded scorer rated axon length on a scale of 1-10, with 10 being a very long axon and 1 being no axon. Standard image analysis software were not used for these quantifications because the variation in axon width interfered with length measurements. Finally, axon branching was quantified by blinded counting of the number of branch sites in each of the images used to score axonal length.

### Statistical Analyses.

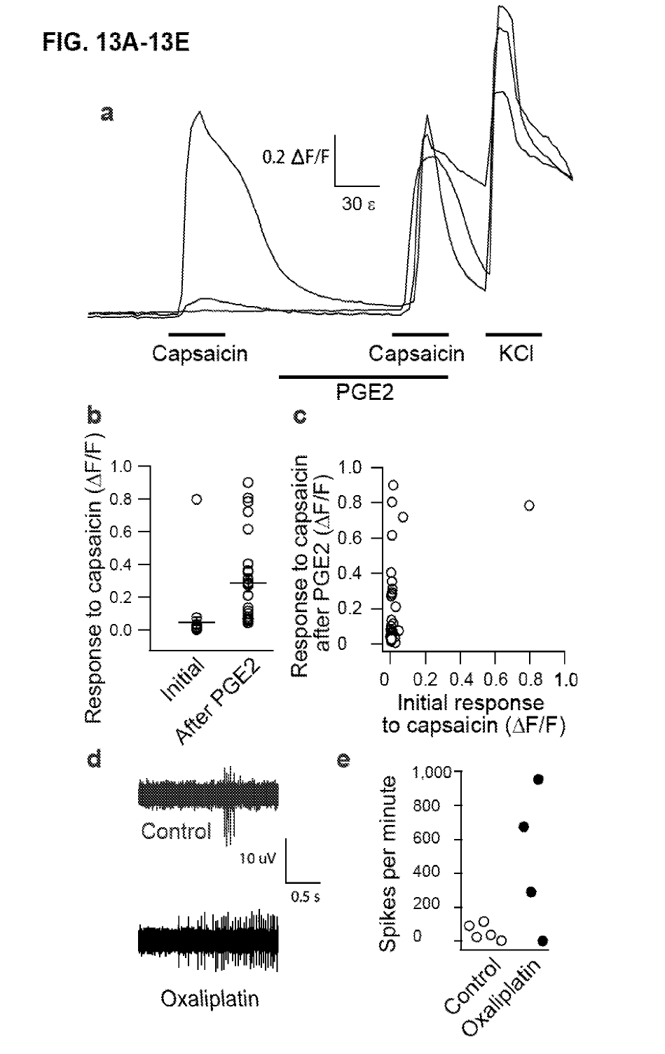

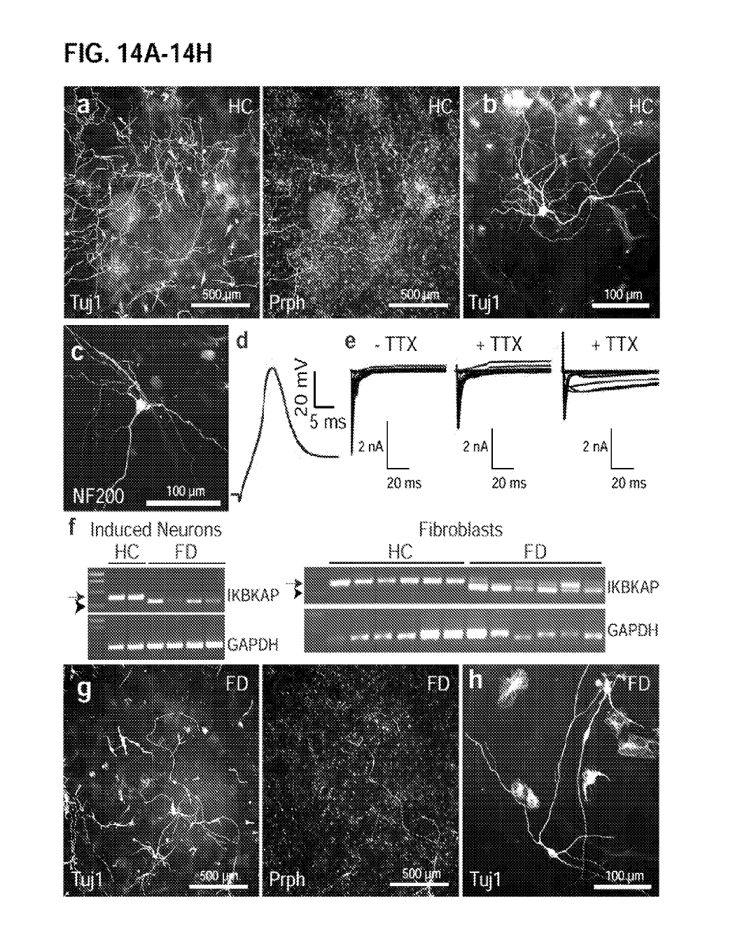

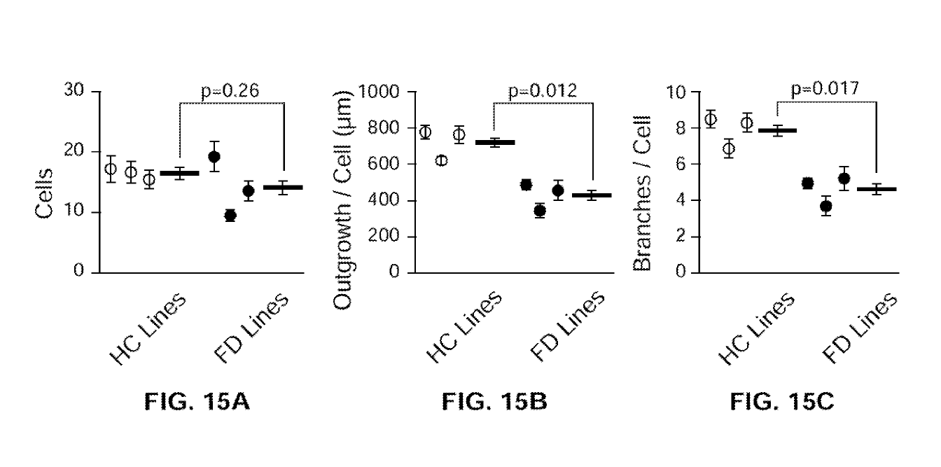

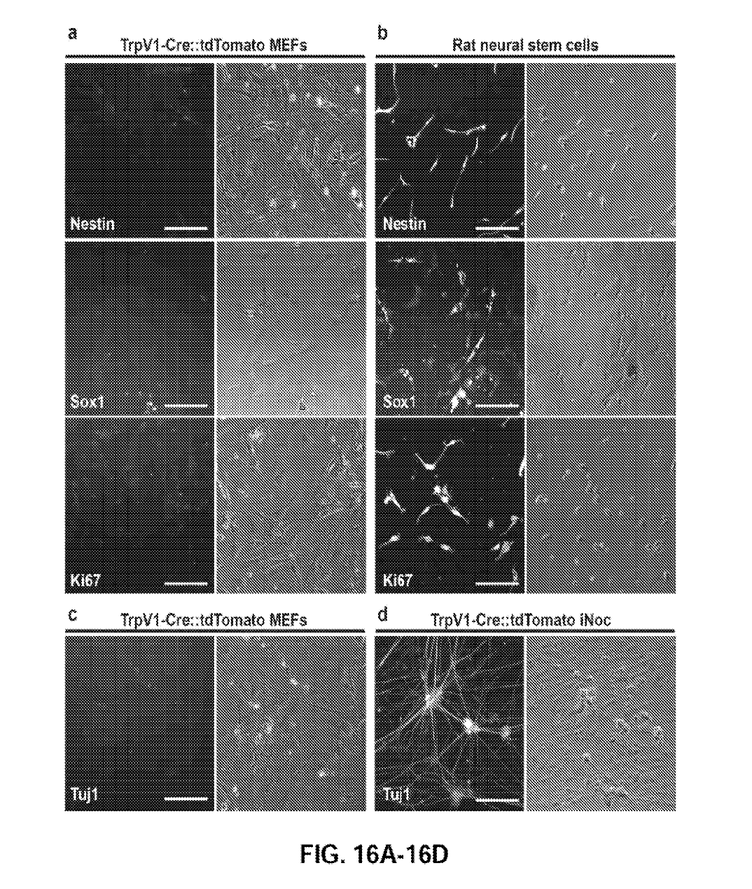

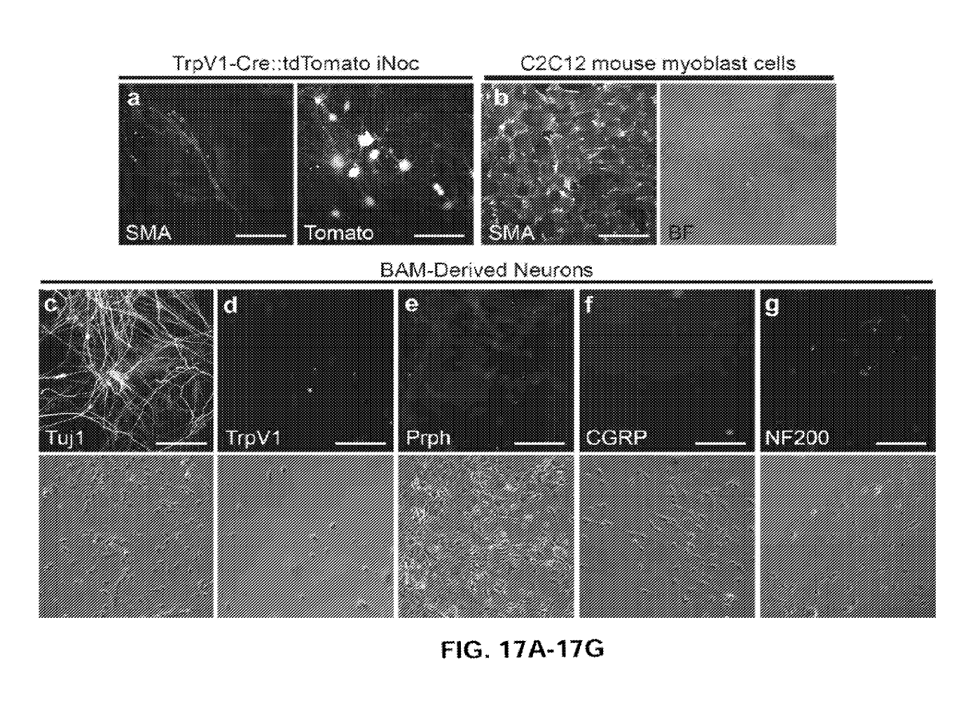

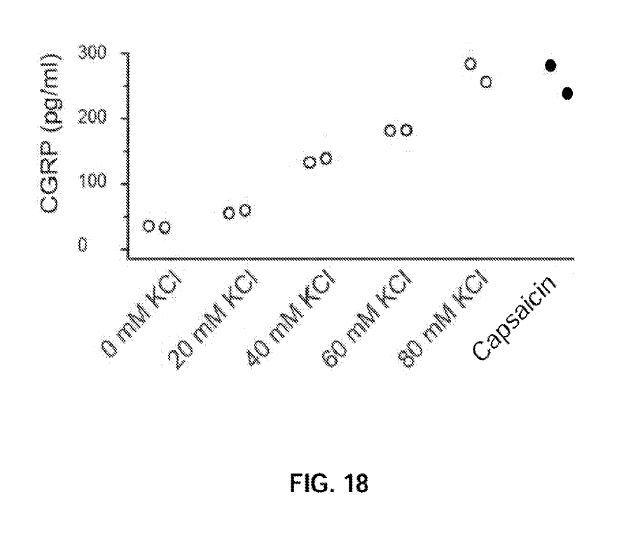

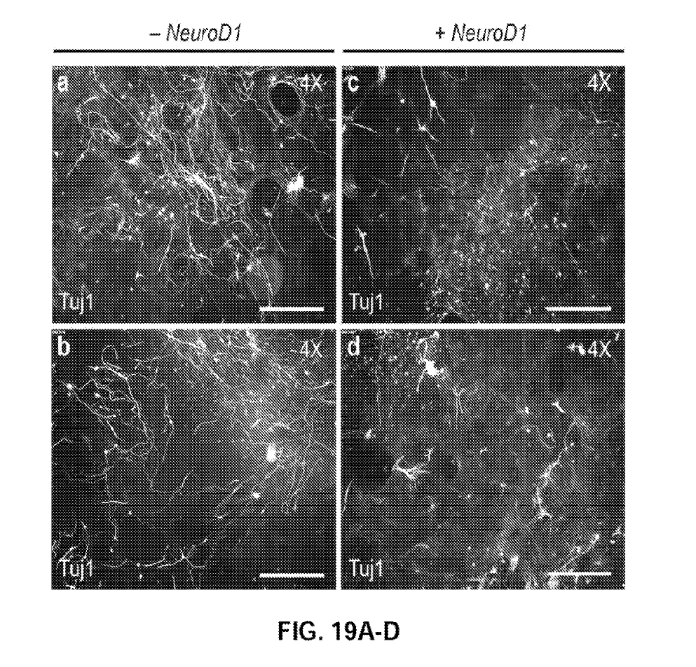

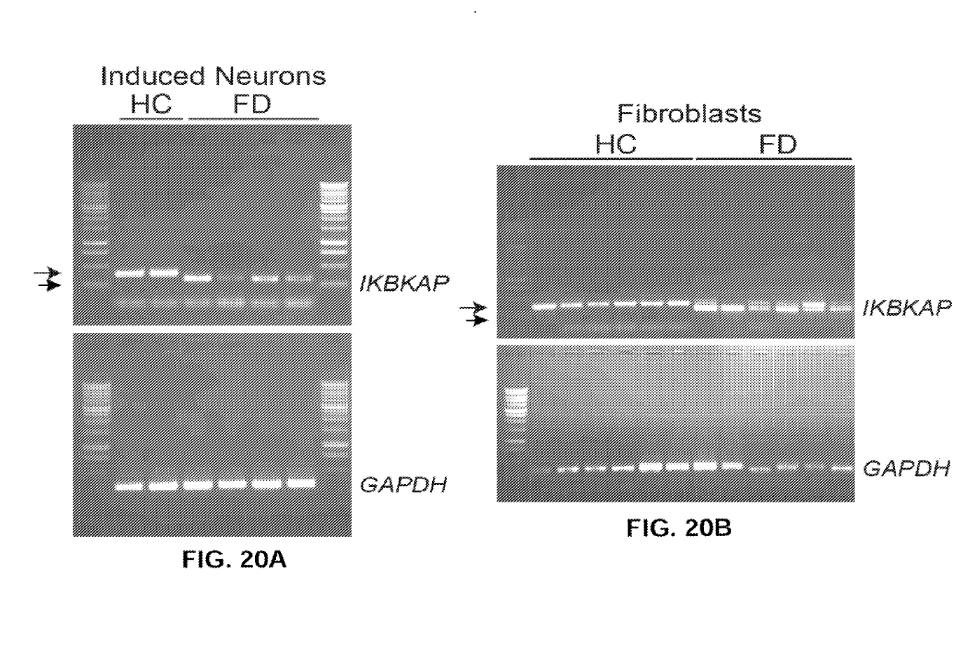

Figures show mean±SEM for all analyses; all tests are two-tailed using a significance threshold of 0.05. A paired t-test was used to evaluate baseline and PGE2-sensitized capsaicin responses in the induced mouse nociceptors (FIGS. 13a-c). This gave a T statistic of 4.61 with 20 degrees of freedom (21 cells) and a p-value of 1.7*10⁻⁴. For morphological analyses of human HC and FD-derived neurons, we performed analyses of cell number, axon length score and number of branch points (FIGS. 14a-f). Distribution of cell number was not normal (Shapiro-Wilk test) and the variance between lines was not equal. The inventors assumed that the distribution of means would be approximately normal based on the central limit theorem and sufficiently large sample size, and used a two-tailed Welch t-test for unequal variance. This gave a T statistic of 3.22 with DF 57.52 and p-value of 2.1*10⁻³. Because a score was used for axonal length, the inventors used a two-tailed Mann Whitney U Test, which gave W=3979.5 and p-value 0.041. Levene's test did not detect a difference in variance between the two lines (p=0.39). Median (IQR) values were 7.5 (6) and 6 (6) for HC and FD neurons. For branch point analysis, the inventors found unequal variance and again relied on central limit theorem and sample number for normality. Two-tailed Welch T-test statistic was 2.87 with DF 106.96 and gave a p-value of 0.005. All statistical analyses were performed in R.

### Example 1

### Selection and Optimization of Transcription Factors

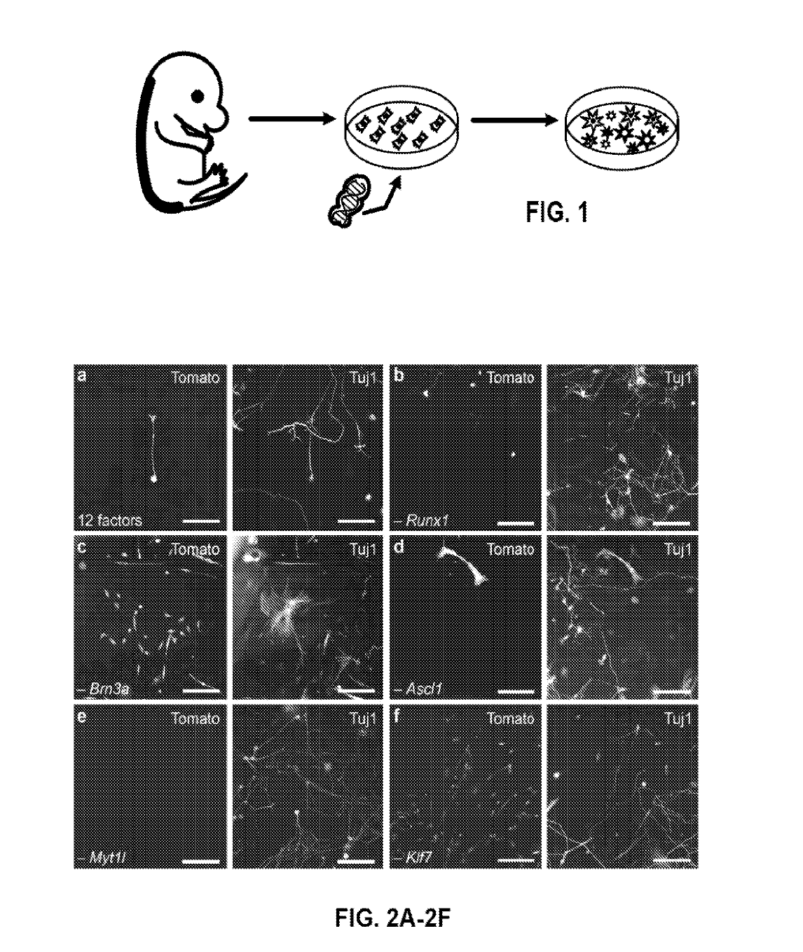

For this study, the inventors first developed nociceptor reporter mice by taking advantage of an existing TrpV1 Cre-driver²⁵ and foxed tdTomato mice to generate TrpV1-Cre^{+/-}:: tdTomato^{+/-} mice, from which we obtained mouse embryonic fibroblasts (MEFs). Thus, activation of the tdTomato reporter would signal the conversion of the MEFs to TrpV1-expressing cells (FIG. 1). To begin, a list of nine transcription factors selected to promote lineage conversion to nociceptors in combination with the BAM factors (12 total, Table 2). These factors were chosen using a combination of prior literature, transcription profiles of FACS-sorted adult mouse nociceptors (NaV1.8-postive) as compared to proprioceptors (parvalbumin-positive) (Chiu et al, submitted), expression profile similarity to NaV1.8 in the BioGPS database²⁶, and dorsal root ganglion (DRG) expression in the Allen Brain Atlas²⁷.

TABLE 2
Candidate transcriptions factors for lineage conversion to nociceptor neurons.

| | | | Role in Reprogramming/Sensory |
|---|---|---|---|
| Gene | Source | Family | System |
| Ascl1 | Lit^{a} | Basic helix-loop-helix/ | Neuronal lineage reprogramming⁷ |
| | | achaete-scute | |
| Drgx | Lit | Helix-turn-helix/Paired | Survival of peptidergic and non- |
| (Drg11) | | box | peptidergic nociceptors⁴⁸ |
| Ebf1 | Exp^{b} | Zinc-finger | Downstream of Ngn⁴⁹ |
| Etv1 | Exp | Helix-turn- | Proprioceptive marker²³ |
| | | helix/tryptophan clusters | |
| Isl2 | Exp, | Homeo-domain/LIM | Unknown |
| | BioGPS^{c} | region | |
| Klf7 | Lit, Exp, | Zinc-finger/Krueppel like | TrkA maintenance²⁴ |
| | BioGPS | | |
| Myt1L | Lit | Zinc-finger | Neuronal lineage reprogramming⁷ |
| Ngn1 | Lit | Basic helix-loop-helix | TrkA and subsequent TrpV1 |
| | | | expression⁴⁷ |
| Pknox2 | Exp | Homeo-domain/TALE | Unknown |
| Pou4fl | Lit | Homeo-domain/POU | Neuronal lineage reprogramming⁷ |
| (Brn3a) | | (Class IV) | |
| Runx1 | Lit | β-scaffold/Runt | Non-peptidergic identify and TrpV1 |
| | | | expression²² |
| Tlx3 | Lit | Helix-turn-helix/homeo- | Glutamatergic identity⁵⁰ |
| | | domain | |

^{a}= literature;
^{b}= transcriptome of sorted nociceptors compared to proprioceptors;
^{c}= BioGPS26.

As expected, there was no baseline activation of the tdTomato reporter in the mouse embryonic fibroblasts Staining of MEFs for neuronal precursor markers using antibodies to Nestin, Sox1, and Ki67, as well as for neuron-specific class III β-tubulin (Tuj1), were all negative (FIG. 16a-d) After transducing the fibroblasts with a combination of all 12 individual retroviruses containing the transcription factors, the inventors detected a small number of tdTomato-positive cells with neuronal morphology after two weeks (FIG. 2a). In order to identify transcription factors that were either critical or inhibitory to the lineage reprogramming into TrpV1-expressing cells, we sequentially eliminated each one at a time. Surprisingly, the elimination of some transcription factors strongly supported by the literature for a role in promoting TrpV1 expression, such as Runx1, did not eliminate TrpV1 reporter expression (FIG. 2b). In fact, elimination of Brn3a led to a marked increase in the number of tdTomato-positive neurons (FIG. 2c). The inventors also identified three factors that were critical to the TrpV1 lineage reprogramming process in that their omission led to a near complete elimination of tdTomato- and neuronal Class III β-tubulin (Tuj1)-positive cells bearing a neuronal morphology: Asc11, Myt11 and Klf7 (FIG. 2d-f).

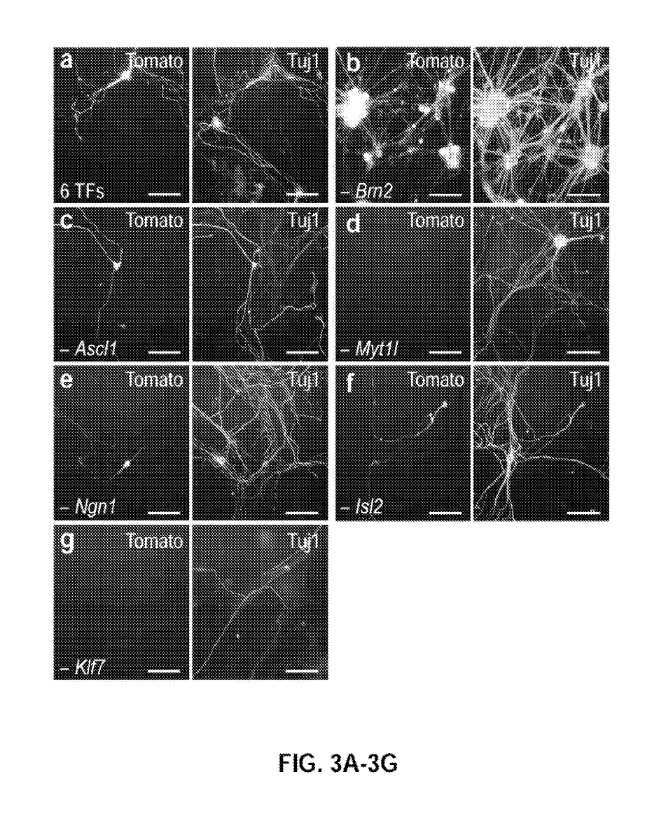

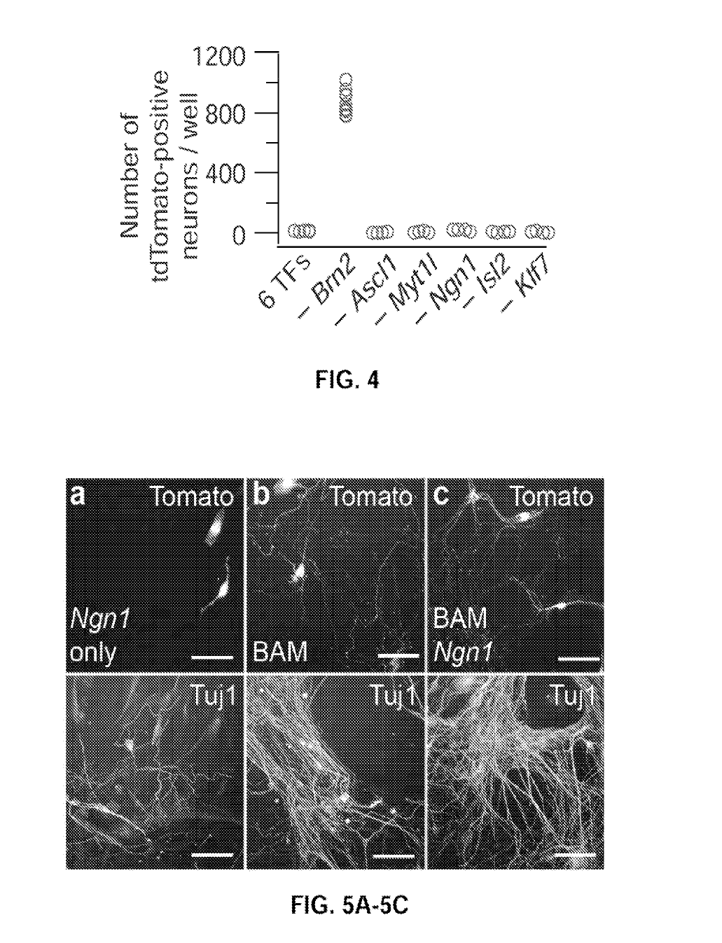

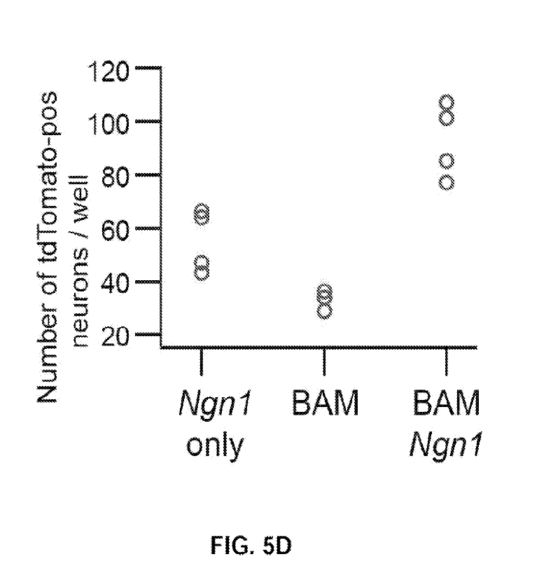

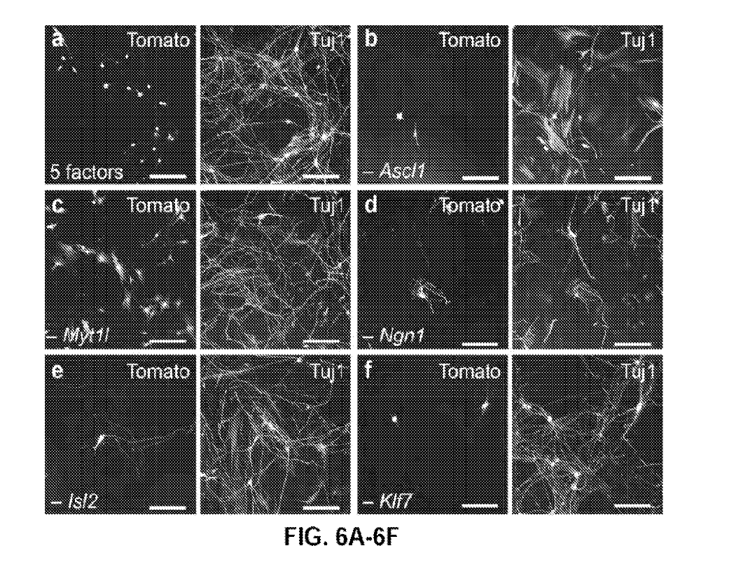

When the three BAM factors were combined with Isl2, Ngn1, and Klf7, only a small number of tdTomato- and Tuj1-postive cells were observed (FIG. 3a). As prior studies⁶ and the inventor's initial drop out experiments detected specific factors that could inhibit the lineage reprogramming process, single factor dropouts from these six factors were performed. The single factor drop out studies demonstrated that elimination of Brn2 led to a striking increase in the number of td-Tomato-positive neurons (FIG. 3b), giving a yield of approximately 14% of plated fibroblasts that were both tdTomato- and Tuj1-positive (less than 0.1% were tdTomato-positive but Tuj1-negative). Removal of any other factor from the six sharply reduced the number of td-Tomato-positive neurons (FIG. 3c-g and FIG. 4). As shown in FIG. 4, the removal of Brn2 markedly increases the number of tdTomato, Tuj1-positive neurons. Omission of Asc11, Myt11, Ngn1, Isl2 or Klf7 from the six factors disrupts the generation of nociceptor neurons. Next, the inventors evaluated Ngn1 alone and in combination with the BAM factors; however, the yield was much lower than with the optimized five factor combination (FIG. 5a-d). Alternative factor combinations generate low number of tdTomato, Tuj1-positve neurons. (FIG. 5a) Ngn1 alone produces a low number of tdTomato, Tuj1-positive cells. (FIG. 5b) the BAM factors produce large numbers of Tuj1-positive cells, a few of which are tdTomato-positive. (FIG. 5c) BAM factors and Ngn1 produce tdTomato, Tuj1-positive neurons, but much less efficiently than the five factors (see FIG. 3a-g and FIG. 4). FIG. 5d demonstrates provides a quantification of the number of tdTomato neurons per well. Indeed, further removal of any of the five factors resulted in a marked decrease in tdTomato, Tuj1-positive cells, demonstrating that the optimized combination of these factors was both necessary and sufficient for reprogramming fibroblasts to a nociceptor fate (FIG. 6a-f). Specifically, FIGS. 6a-f demonstrate that removal of any of the five factors disrupts nociceptor formation. (a) Transduction of MEFs with all 5 factors (Asc11, Myt11, Ngn1, Isl2, and Klf7) efficiently produce tdTomato, Tuj1-positive neurons. (b-f) Removal of Asc11 (b), Myt11 (c), Ngn1 (d), Isl2 (e), or Klf7 (f) dramatically reduces the number of tdTomato, Tuj1-positive neurons.

### Example 2

### Molecular Characterization of Induced Mouse Nociceptors

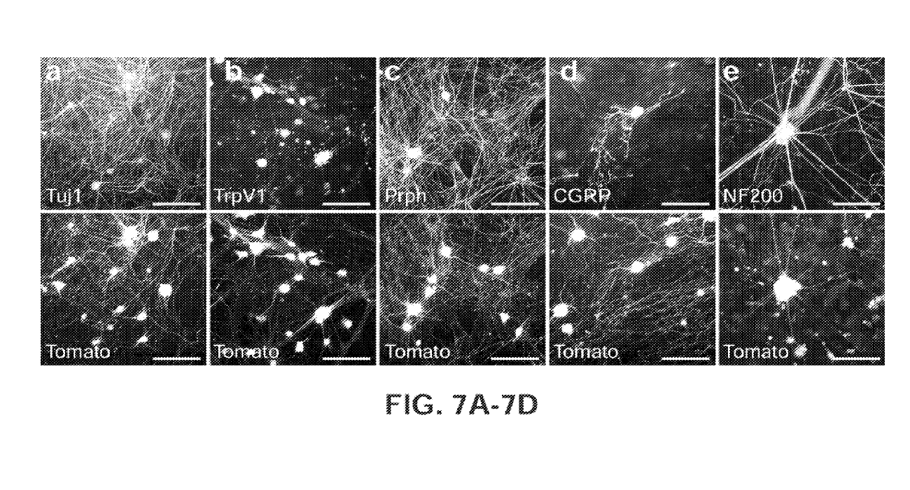

To determine the similarity of tdTomato-positive reprogrammed neurons and bona fide nociceptors, the inventors evaluated the expression of proteins specific for nociceptor neurons. Nearly all tdTomato-positive neurons stained for the pan-neuronal marker Tuj1 and had a neuronal-like morphology with many long branching axons, and most Tuj1-positive neurons were tdTomato-positive (FIG. 7a). Staining with an anti-TrpV1 antibody confirmed the translation of the TrpV1 protein in the vast majority of tdTomato-positive neurons (FIG. 7b). In mouse dorsal root ganglia, most TrpV1-expressing neurons are C-fibers that express the marker peripherin (Prph)²⁸, while a small percentage of A-δ fibers are also TrpV1-positive²⁵. In the induced neurons, most tdTomato-positive neurons expressed peripherin (66.9±4.1%, n=16 wells from 4 separate transductions) (FIG. 7c), and many CGRP (22.3±6.6%, n=4 wells from 2 separate transductions) (FIG. 7d), however a small number of cells stained for the intermediate filament NF200, a marker of A-δ nociceptors (FIG. 7e). In contrast, the derived nociceptors did not stain for smooth muscle actin (SMA), a marker of muscle, despite reports of TrpV1 expression in muscle30 (FIGS. 17a, b). Furthermore, neurons derived from the three BAM factors did not express nociceptor markers, consistent with their high specificity FIGS. 17c-g).

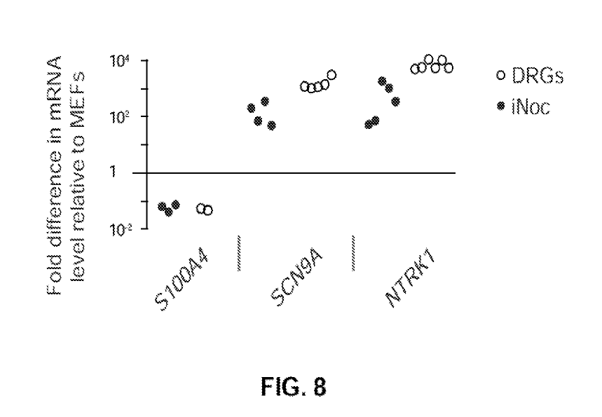

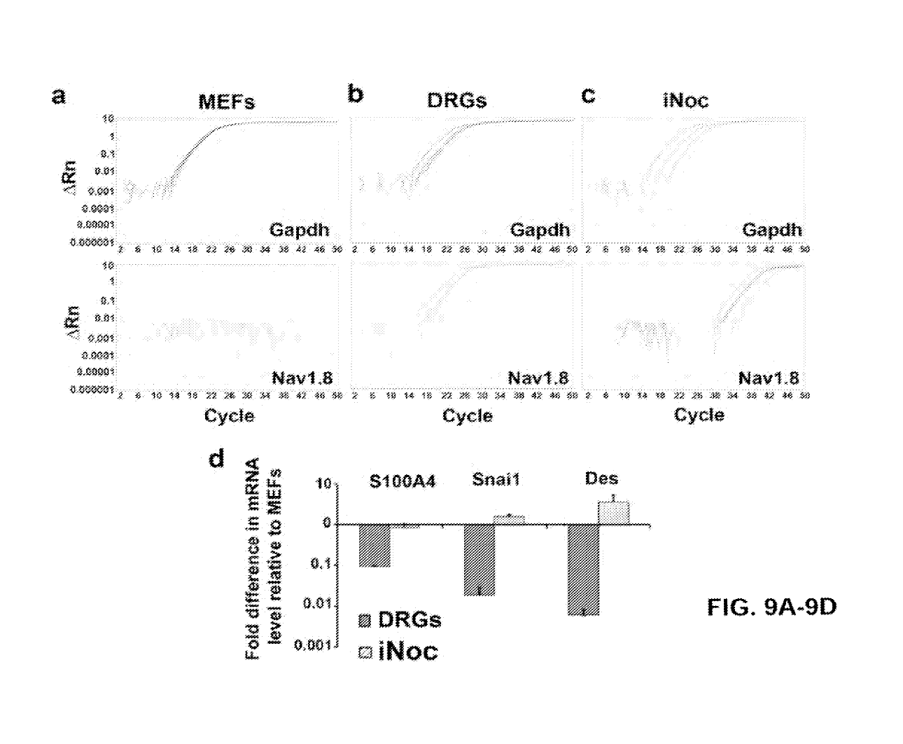

Because specific antibodies do not exist for many of the quintessential nociceptor proteins, we utilized quantitative RT-PCR to compare the mRNA levels in FACS-sorted tdTomato-positive induced nociceptors and FACS-sorted tdTomato-positive adult mouse nociceptors relative to levels in the TrpV1-Cre::tdTomato MEFs (FIG. 8). For this analysis, we used patch pipettes to pick tdTomato-positive induced and primary mouse neurons, as well as MEFs, and plotted the levels of specific transcripts in induced and primary nociceptors relative to MEFs. The fibroblast marker S100A4 was expressed at a similar very low level in both the induced and primary nociceptors, consistent with a non-fibroblast identity of the induced nociceptors. NaV1.7 (Scn9a), which is found in nociceptor and autonomic peripheral neurons, was present in both the induced and primary nociceptors, as was TrkA (NTRK1), which is turned on in developing nociceptors and persists in the peptidergic subset of mature nociceptors, although the expression of NaV1.7 and TrkA in the induced neurons was several fold less than in the primary DRGs. Transcripts for the C-fiber marker peripherin were present in the induced neurons, confirming the immunostaining results. TrkA, which is turned on in developing nociceptors and persists in the peptidergic subset of mature nociceptors, was detected as was the peptidergic transmitter CGRP. RNAs for the nociceptor-specific Trp channels, TrpV1, TrpA1 and TrpM8, were all present, as well as transcripts for the nociceptor-specific P2X3 purinergic receptor′. NaV1.7, which is found in nociceptor and autonomic peripheral neurons, was present, as was the NaV1.9 TTX-resistant sodium channel. In general, nociceptor-specific RNA transcript levels were consistently higher for the induced neurons than for MEFs, but not as high as for the primary nociceptors. The MEFs did not yield any appreciable NaV1.8 DNA signal, even after 50 RT-PCR cycles, and consequently we could not plot the relative levels; however, NaV1.8 transcripts were detected in the induced neurons (FIGS. 9a-c). Interestingly, the induced neurons did not down-regulate some key MEF genes, suggesting that they still maintain some MEF identity (FIG. 9d). Together, these immunohistochemistry and PCR data suggest that the induced neurons express a compliment of bona fide nociceptor-specific markers.

### Example 3

### Functional Properties of Induced Mouse Nociceptors

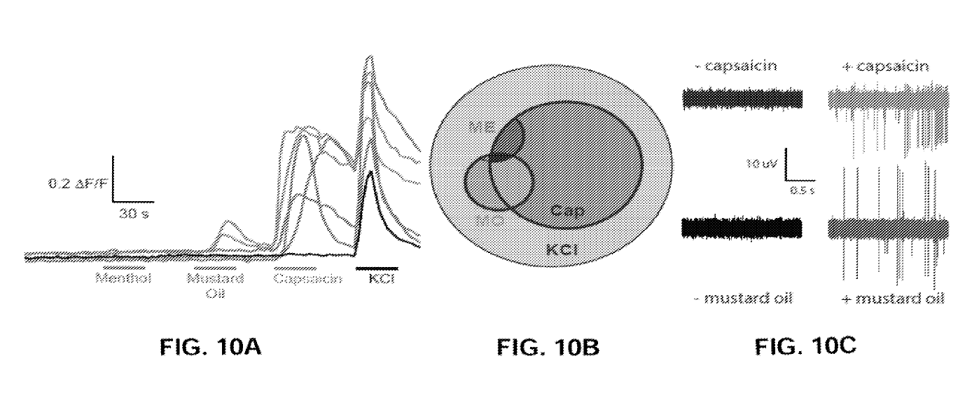

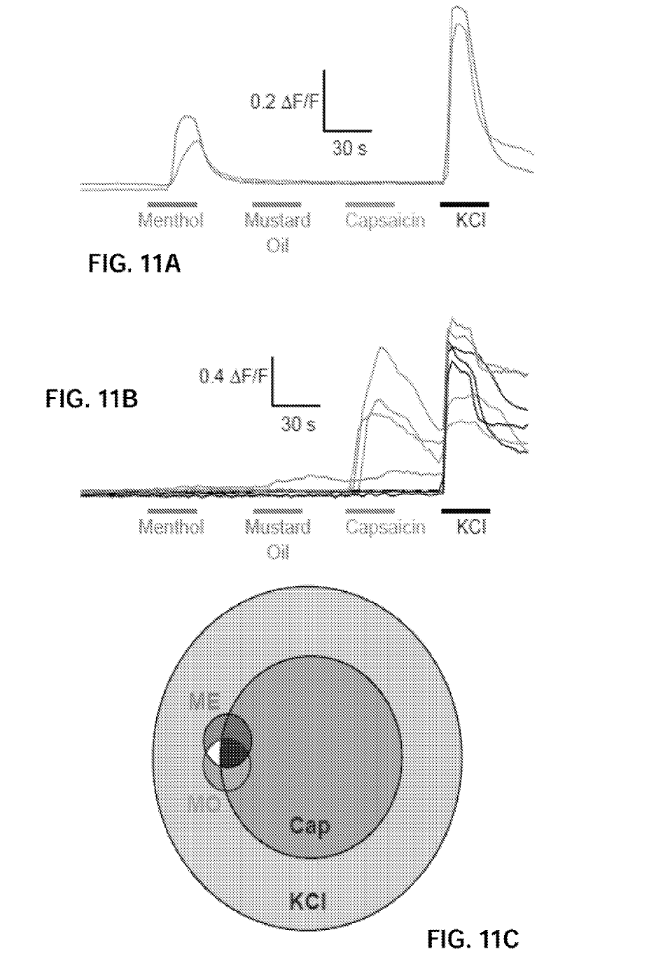

In order to investigate the functional properties of the induced nociceptors, the inventors performed calcium imaging with a battery of agonists and evaluated the number of responders within the tdTomato-positive population with a stable baseline and response to potassium chloride (KCl, which activates voltage-gated calcium channels through depolarization and serves as a measure of neuronal functional integrity) (FIG. 10a). The inventors chose concentrations of TrpM8 (250 μM menthol), TrpA1 (100 μM mustard oil) and TrpV1 (1 μM capsaicin) agonists that activated single receptors and did not exhibit receptor cross-reactivity³⁰. 39% of tdTomato-positive cells responded to capsaicin and 9% responded to mustard oil and 3% responded to menthol (FIG. 10a, b; n=227 tdTomato-positive cells that responded to KCl). The inventors observed occasional cells that responded to both mustard oil and capsaicin, a single cell that responded to menthol and mustard oil but not capsaicin, and one cell that responded to all three agonists. The inventors did not observe any tdTomato-positive cells that responded to menthol alone, but a small number of tdTomato-negative cells that responded to menthol but not the other Trp agonists were identified (FIG. 11a). In contrast, 0/50 KCl-responding neurons derived from the BAM factors alone responded to capsaicin (not shown). Using the same experimental procedure, the inventors then investigated then asked how the frequencies of the different combinations of receptors within individual neurons compared between induced nociceptors and adult mouse nociceptors. In tdTomato-positive primary DRG neurons dissected and cultured from adult TrpV1::tdTomato mice, 36% of the neurons responded to capsaicin, 2.5% to mustard oil and 2.5% to menthol (FIGS. 11b,c; n=249 tdTomato-positive cells that responded to KCl). Thus, the nociceptor lineage reprogramming not only yielded physiologically functional TrpV1, TrpA1 and TrpM8 proteins in the induced neurons, but the frequencies and combinations of the different receptors in the induced neurons closely mimicked those of adult mouse nociceptors.

While calcium imaging provides detailed information about calcium entry through Trp channels, it may not necessarily reflect the action potential firing properties of the neurons. By culturing the induced neurons on extracellular multi-electrode arrays, the inventors found that capsaicin and mustard oil application yielded robust action potential firing in the induced neurons (FIG. 10c; 3/3 arrays for capsaicin and 3/3 arrays for mustard oil).

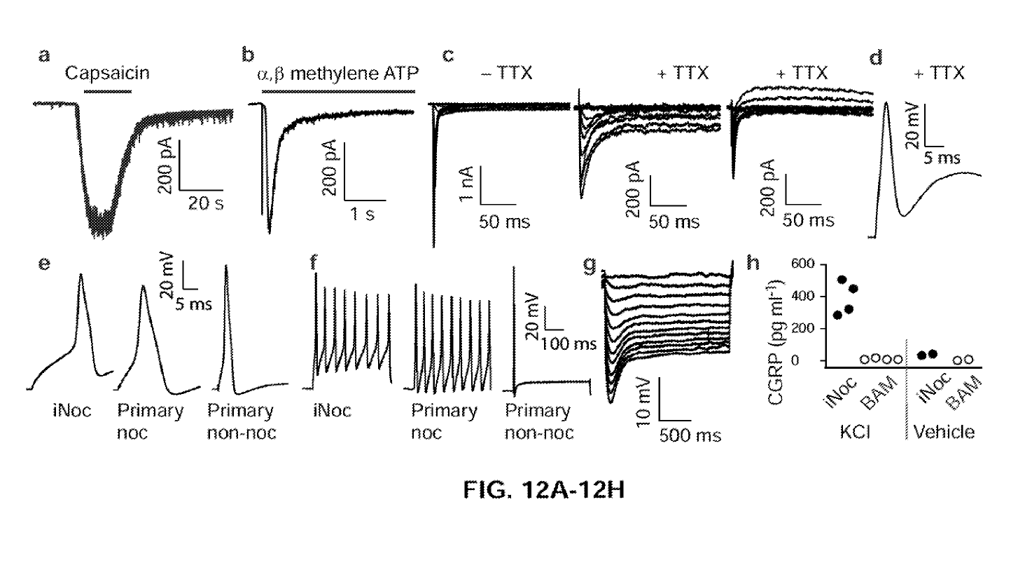

Next, whole-cell patch clamp was used to define the electrophysiological properties of the reprogrammed nociceptors. Using patch clamp, the inventors found that 1 μM capsaicin elicited inward currents in tdTomato-positive induced neurons in 6/11 neurons, consistent but somewhat higher than the responding percentage in the calcium imaging results (FIG. 12a). In addition to the different Trp channels, the P2X3 subtype of ionotropic purinergic receptors is expressed specifically in nociceptor neurons^{29,31,32}. Application of the P2X3-specific agonist α, β-methylene-ATP (30 μM) elicited rapidly-adapting inward currents in 8/16 neurons (FIG. 12b) that were blocked completely by A-397491, a specific P2X3 antagonist in 4/4 neurons³³.

Perhaps the most nociceptor-specific marker is the TTX-resistant NaV1.8 sodium channel, which produces the majority of the current in the nociceptor action potential upstroke³⁴. In voltage-clamp, depolarizing voltage steps elicited inward sodium currents before and after the application of 300 nM TTX (FIG. 12c; 14/15 recorded induced nociceptors had TTX-resistant sodium currents greater than 50 pA). Consistent with the expression studies, the slow channel kinetics of the TTX-resistant currents are typical for NaV1.8 as opposed to the fast NaV1.6 cardiac sodium channel, which has been found to be present in developing embryonic nociceptors³⁵. Furthermore, five of the 14 neurons with TTX-resistant sodium currents also exhibited a persistent sodium component, which previous studies have found to be due to NaV1.9 (FIG. 4c). The ability to generate action potentials in the presence of TTX is a feature of nociceptors but not of other DRG or central neurons. The induced neurons fired single TTX-resistant action potentials that overshot 0 mV in 8/16 neurons (FIG. 12d). NaV1.8 is responsible for the characteristic broad action potential shape of the nociceptor action potential¹², which we found to be a property of a subset of induced neurons (mean action potential width 3.32±0.33 ms; n=13) and adult primary tdTomato-positive nociceptors compared to large tdTomato-negative non-nociceptor DRG neurons (FIG. 12e). In addition to differences in action potential morphology, the firing pattern of nociceptor neurons is tonic, compared to the phasic firing of most large A-β DRG neurons³⁶. Induced nociceptors fired tonic action potential trains in response to depolarizing current steps, consistent with the tonic firing in tdTomato-positive primary mouse nociceptors, but in contrast to the single action potentials elicited in non-nociceptor, large tdTomato-negative adult DRG neurons (FIG. 12f).

While hyperpolarization-activated cyclic nucleotide-sensitive (HCN) currents are not specific for nociceptor neurons, they play an important role in neuropathic and inflammatory pain³⁷, and thus their presence may be important for disease-modeling. We found that the induced nociceptors produced typical sag depolarizations in response to hyperpolarization (FIG. 12g) in 11/17 tdTomato-positive induced neurons, consistent with ZD7288-sensitive HCN currents recorded in voltage clamp (2/2, not shown).

A critical function of peptidergic neurons, most of which express TrpV17, is to release neuropeptides such as CGRP and Substance P. To assess the fidelity of the induced nociceptors in this capacity, we measured CGRP levels in supernatant following a depolarizing stimulus and found that induced nociceptors, but not BAM-derived neurons, released CGRP after KCl stimulation (FIG. 12h; n=4; Mann-Whitney U-test p=0.03). The concentrations of CGRP released by the induced neurons were comparable to those released by primary DRG neurons (FIG. 18), thus indicating that the induced neurons have synaptic vesicle release mechanisms in place.

### Example 4

### Induced Nociceptors Model Inflammatory Sensitization

The transition from high-threshold baseline nociception to low-threshold clinical pain hypersensitivity commonly involves peripheral sensitization of nociceptors. For the induced nociceptors to be valuable in vitro models of in vivo pathophysiology, they must replicate not only the specific functional molecular channels and receptors of the cells but also the process of sensitization that leads to pathological pain. Prostaglandin E2 (PGE2) activates the PKA pathway and sensitizes the TrpV1 receptor^{38,39}. In the tdTomato-positive induced neurons, a lower concentration (300 nM) of capsaicin rarely yielded a detectable response (mean change in fluorescence absorption ratio of 0.028±3.0*10⁻³) (FIG. 13a, b). However, after treatment with 1 μM PGE2 for two minutes, a second 300 nM capsaicin application yielded a mean response of 0.18±6.0*10⁻³ (n=41; paired t-test p=1.7×10⁻⁴). Plotting the magnitudes of the initial capsaicin and PGE2-sensitized capsaicin responses revealed that the majority of neurons exhibited small or undetectable initial responses to capsaicin but robust signals after PGE2 sensitization (FIG. 13c).

TrpV1 sensitization also may contribute to painful chemotherapy-induced neuropathy due to oxaliplatin. Using MEA recording, we compared capsaicin responses in induced nociceptors treated with either 50 μM oxaliplatin or vehicle control, and found marked sensitization in the oxaliplatin-treated nociceptors (FIGS. 13d, e)

### Example 5

### Induction of Human Nociceptors

To derive nociceptors from human fibroblasts, the inventors included NeuroD1 in the nociceptor induction protocol, as this transcription factor was important for prior human lineage reprogramming studies⁴⁰. However, the inventors found that the reprogramming efficiency, was greater without NeuroD1 (five factors) than with NeuroD1 (six factors) (20.7±1.4 cells per field for five factors; 9.7±1.1 cells per field for six factors, n=6 wells/group; t-test p=1.0×10-4) (FIG. 19a-d). Furthermore, more neurons exhibited larger sodium currents (67% of patched five factor neurons had peak transient sodium currents greater than 500 pA, versus 29% of six factor neurons) and five factor neurons were healthier (resting Vm −49.3±2.2 mV, n=33 five factor neurons; Vm −37.3±3.2, n=20 six factor neurons; Mann-Whitney U-test p-value=0.001). Using healthy control (HC) subject fibroblasts, the 5 factors yielded Tuj1-positive neurons at an efficiency of 5% of plated fibroblasts, and 16% of the Tuj1-positive neurons were also peripherin-positive (FIG. 14a,b), efficiencies that were somewhat lower than the mouse induced nociceptors. A small number of the Tuj1-positive neurons were NF200-positive (FIG. 14c). We recorded from the neurons using whole-cell patch clamp. Although we did not have a reporter for a particular neuronal subtype, the induced human neurons fired broad action potentials (mean action potential width 3.88±0.41 ms; n=17; FIG. 15d), consistent with functional nociceptors. In 38 voltage clamp recordings, we applied TTX to neurons with a large total sodium current (greater than 1 nA) and detected TTX-resistant sodium currents in 10/10 neurons (FIG. 14e). As in both our mouse induced nociceptors and primary mouse and human nociceptors³⁴, the induced human neurons had different combinations of slow- and persistent TTX-resistant sodium currents, consistent with NaV1.8 and NaV1.9 contributions, respectively (FIG. 14e).

In order to evaluate the potential of the human neurons for disease modeling, the reprogrammed fibroblasts from three HC and three unrelated, age-matched subjects with familial dysautonomia (FD, hereditary sensory and autonomic neuropathy type III, Riley-Day syndrome), due to a homozygous donor splice site mutation that results in deletion of intron 20 from the I-κ-β kinase complex-associated protein (IKBKAP) RNA′. Single FD-derived neurons picked using patch pipettes exclusively expressed the abnormally spliced transcript, something not previously identified, while the HC-derived neurons expressed only the normal transcript (FIG. 14f). FD fibroblasts expressed a mixture of abnormally spliced and normal transcripts, consistent with prior studies^{51,52}, while HC fibroblasts expressed only the normal transcript (FIG. 14f; FIG. 20).

Although we detected peripherin-positive, Tuj1-positive neurons from all HC and FD subjects (FIG. 14g,h), the neurons from FD subjects showed a trend toward decrease in number (FIG. 16a; 16.5±1.1 HC neurons/well, n=60 wells; 14.1±1.1 FD neurons/well, n=60 wells; difference between HC neurons/well and FD neurons/well 2.3±1.5, n=60 wells; random intercept mixed-effects model p=0.26) and a robust reduction in neurite outgrowth per cell (FIG. 16b; 725±24 μm per HC neuron, n=60 wells; 433±25 μm per FD neuron, n=60 wells; difference between HC neuron outgrowth per cell/well and FD neuron outgrowth per cell/well 291.3±32.6 μm, n=60 wells; random intercept mixed-effects model p=0.012), as well as number of branches per neuron (FIG. 16c; 7.9±0.3 branches per HC neuron, n=60; 4.7±0.3 branches per FD neuron, n=60 wells; difference between HC braches per neuron/well and FD branches per neuron/well 3.3±0.4, n=60 wells; random intercept mixed-effects model p=0.017) compared to HC-derived neurons.

The examples above demonstrate that small number of transcription factors can relatively efficiently convert fibroblasts into neurons that express the key specific functional receptors found in bona fide adult nociceptors. While TrpV1 is expressed in a tiny fraction of central neurons45, NaV1.8 and TrpA1 are not expressed within the central nervous system. The collective expression of subsets of these markers defines specific subpopulations, and indeed to a first approximation the neurons generated above recreate the combinatorial patterns that define the diversity of TrpV1-expressing nociceptive neuronal cohorts. The examples appear not to have derived a single nociceptor type but instead engineered cells of multiple subtypes similar to those found in vivo.

The intricate physiology of primary nociceptor neurons and the fortunate ability to culture adult sensory neurons provide an unusual and well-controlled opportunity to evaluate how closely lineage-reprogrammed neurons model the functional receptors and channels of primary adult neurons. The inventors have found that the reprogrammed neurons produced functional TrpV1, TrpA1 and TrpM8-expressing neurons in similar percentages as compared to primary tdTomato-positive adult mouse neurons. Similarly, the induced neurons produce not only functional TTX-resistant action potentials, but the broad action potential morphology and phasic firing pattern that are characteristic of nociceptors, as opposed to non-nociceptor DRG neurons.

Patient-derived neurons will have great utility as a drug screening tool if the derived neurons model not only disease-relevant cell types but also the sequence of pathophysiological changes that underlie specific clinical diseases. The reprogrammed nociceptors may be particularly useful as an in vitro model for chronic pain, because the pain sensitization process mimicked by the induced nociceptors drives the transition from baseline nociception to pathological chronic pain. The examples provided herein illustrate how the derived neurons may be generated and employed as an in vitro model for pain in a dish and in the future for a screening platform to identify new analgesics.

Throughout this application, various publications are referenced. The disclosures of all of the publications and those references cited within those publications in their entireties are hereby incorporated by reference into this application in order to more fully describe the state of the art to which this invention pertains.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.
1 Qiang, L., Fujita, R. & Abeliovich, A. Remodeling neurodegeneration: somatic cell reprogramming-based models of adult neurological disorders. Neuron 78, 957-969 (2013).
2 Sandoe, J. & Eggan, K. Opportunities and challenges of pluripotent stem cell neurodegenerative disease models. Nat Neurosci 16, 780-789 (2013).
3 Zhou, Q., Brown, J., Kanarek, A., Rajagopal, J. & Melton, D. A. In vivo reprogramming of adult pancreatic exocrine cells to beta-cells. Nature 455, 627-632 (2008).
4 Liu, M. L. et al. Small molecules enable neurogenin 2 to efficiently convert human fibroblasts into cholinergic neurons. Nat Commun 4, 2183 (2013).
5 Liu, X. et al. Direct reprogramming of human fibroblasts into dopaminergic neuron-like cells. Cell Res 22, 321-332 (2012).
6 Son, E. Y. et al. Conversion of mouse and human fibroblasts into functional spinal motor neurons. Cell Stem Cell 9, 205-218 (2011).
7 Vierbuchen, T. et al. Direct conversion of fibroblasts to functional neurons by defined factors. Nature 463, 1035-1041 (2010).
8 Basbaum, A. I., Bautista, D. M., Scherrer, G. & Julius, D. Cellular and molecular mechanisms of pain. Cell 139, 267-284 (2009).
9 Hucho, T. & Levine, J. D. Signaling pathways in sensitization: toward a nociceptor cell biology. Neuron 55, 365-376 (2007).
10 Costigan, M., Scholz, J. & Woolf, C. J. Neuropathic pain: a maladaptive response of the nervous system to damage. Annu Rev Neurosci 32, 1-32 (2009).
11 Hughes, J. P. et al. Understanding chronic inflammatory and neuropathic pain. Ann N Y Acad Sci 1255, 30-44 (2012).
12 Blair, N. T. & Bean, B. P. Roles of tetrodotoxin (TTX)-sensitive Na+ current, TTX-resistant Na+ current, and Ca2+ current in the action potentials of nociceptive sensory neurons. J Neurosci 22, 10277-10290 (2002).
13 Chambers, S. M. et al. Combined small-molecule inhibition accelerates developmental timing and converts human pluripotent stem cells into nociceptors. Nat Biotechnol 30, 715-720 (2012).
14 Novella, S. P., Hisama, F. M., Dib-Hajj, S. D. & Waxman, S. G. A case of inherited erythromelalgia. Nat Clin Pract Neurol 3, 229-234 (2007).
15 Huang, J. et al. Small-fiber neuropathy Nav1.8 mutation shifts activation to hyperpolarized potentials and increases excitability of dorsal root ganglion neurons. J Neurosci 33, 14087-14097 (2013).
16 Han, C. et al. Nav1.7-related small fiber neuropathy: impaired slow-inactivation and DRG neuron hyperexcitability. Neurology 78, 1635-1643 (2012).
17 Faber, C. G. et al. Gain of function Nanu1.7 mutations in idiopathic small fiber neuropathy. Ann Neurol 71, 26-39 (2012).
18 Lee, K. J. & Jessell, T. M. The specification of dorsal cell fates in the vertebrate central nervous system. Annu Rev Neurosci 22, 261-294 (1999).
19 Knecht, A. K. & Bronner-Fraser, M. Induction of the neural crest: a multigene process. Nat Rev Genet 3, 453-461 (2002).
20 Woolf, C. J. & Ma, Q. Nociceptors—noxious stimulus detectors. Neuron 55, 353-364 (2007).
21 Dykes, I. M., Lanier, J., Eng, S. R. & Turner, E. E. Brn3a regulates neuronal subtype specification in the trigeminal ganglion by promoting Runx expression during sensory differentiation. Neural Dev 5, 3 (2010).
22 Chen, C. L. et al. Runx1 determines nociceptive sensory neuron phenotype and is required for thermal and neuropathic pain. Neuron 49, 365-377 (2006).
23 Sun, Y. et al. A central role for Islet1 in sensory neuron development linking sensory and spinal gene regulatory programs. Nat Neurosci 11, 1283-1293 (2008).
24 Lei, L. et al. The zinc finger transcription factor Klf7 is required for TrkA gene expression and development of nociceptive sensory neurons. Genes Dev 19, 1354-1364 (2005).
25 Cavanaugh, D. J. et al. Restriction of transient receptor potential vanilloid-1 to the peptidergic subset of primary afferent neurons follows its developmental downregulation in nonpeptidergic neurons. J Neurosci 31, 10119-10127 (2011).
26 Wu, C., Macleod, I. & Su, A. I. BioGPS and MyGene.info: organizing online, gene-centric information. Nucleic Acids Res 41, D561-565 (2013).
27 Website: ©2012 Allen Institute for Brain Science. Allen Spinal Cord Atlas [Internet]. Available from: http://mousespinal.brain-map.org/.
28 Fornaro, M. et al. Neuronal intermediate filament expression in rat dorsal root ganglia sensory neurons: an in vivo and in vitro study. Neuroscience 153, 1153-1163 (2008).
29 North, R. A. Molecular physiology of P2X receptors. Physiol Rev 82, 1013-1067 (2002).
30 Bautista, D. M. et al. TRPA1 mediates the inflammatory actions of environmental irritants and proalgesic agents. Cell 124, 1269-1282 (2006).
31 Cook, S. P., Vulchanova, L., Hargreaves, K. M., Elde, R. & McCleskey, E. W. Distinct ATP receptors on pain-sensing and stretch-sensing neurons. Nature 387, 505-508 (1997).
32 Chen, C. C. et al. A P2X purinoceptor expressed by a subset of sensory neurons. Nature 377, 428-431 (1995).
33 Jarvis, M. F. et al. A-317491, a novel potent and selective non-nucleotide antagonist of P2X3 and P2X2/3 receptors, reduces chronic inflammatory and neuropathic pain in the rat. Proc Natl Acad Sci USA 99, 17179-17184 (2002).
34 Dib-Hajj, S. D., Cummins, T. R., Black, J. A. & Waxman, S. G. Sodium channels in normal and pathological pain. Annu Rev Neurosci 33, 325-347 (2010).
35 Renganathan, M., Dib-Hajj, S. & Waxman, S. G. Na(v)1.5 underlies the ‘third TTX-R sodium current’ in rat small DRG neurons. Brain Res Mol Brain Res 106, 70-82 (2002).
36 Nagy, I., Urban, L. & Woolf, C. J. Morphological and membrane properties of young rat lumbar and thoracic dorsal root ganglion cells with unmyelinated axons. Brain Res 609, 193-200 (1993).
37 Emery, E. C., Young, G. T., Berrocoso, E. M., Chen, L. & McNaughton, P. A. HCN2 ion channels play a central role in inflammatory and neuropathic pain. Science 333, 1462-1466 (2011).
38 Pitchford, S. & Levine, J. D. Prostaglandins sensitize nociceptors in cell culture. Neurosci Lett 132, 105-108 (1991).
39 Lopshire, J. C. & Nicol, G. D. Activation and recovery of the PGE2-mediated sensitization of the capsaicin response in rat sensory neurons. J Neurophysiol 78, 3154-3164 (1997).
40 Pang, Z. P. et al. Induction of human neuronal cells by defined transcription factors. Nature 476, 220-223 (2011).
41 Slaugenhaupt, S. A. et al. Tissue-specific expression of a splicing mutation in the IKBKAP gene causes familial dysautonomia. Am J Hum Genet 68, 598-605 (2001).
42 Woolf, C. J. Overcoming obstacles to developing new analgesics. Nat Med 16, 1241-1247 (2010).
43 Cox, J. J. et al. An SCN9A channelopathy causes congenital inability to experience pain. Nature 444, 894-898 (2006).
44 Nassar, M. A. et al. Nociceptor-specific gene deletion reveals a major role for Nav1.7 (PN1) in acute and inflammatory pain. Proc Natl Acad Sci USA 101, 12706-12711 (2004).
45 Cavanaugh, D. J. et al. Trpv1 reporter mice reveal highly restricted brain distribution and functional expression in arteriolar smooth muscle cells. J Neurosci 31, 5067-5077 (2011).
46 Li, Y. et al. Cloning and expression of a novel human gene, Isl-2, encoded a LIM-homeodomain protein. Mol Biol Rep 34, 19-26 (2007).
47 Ma, Q., Fode, C., Guillemot, F. & Anderson, D. J. Neurogenin1 and neurogenin2 control two distinct waves of neurogenesis in developing dorsal root ganglia. Genes Dev 13, 1717-1728 (1999).
48 Rebelo, S., Chen, Z. F., Anderson, D. J. & Lima, D. Involvement of DRG11 in the development of the primary afferent nociceptive system. Mol Cell Neurosci 33, 236-246 (2006).
49 Anderson, D. J. Lineages and transcription factors in the specification of vertebrate primary sensory neurons. Curr Opin Neurobiol 9, 517-524 (1999).
50 Cheng, L. et al. Tlx3 and Tlx1 are post-mitotic selector genes determining glutamatergic over GABAergic cell fates. Nat Neurosci 7, 510-517 (2004).
51. Cuajungco, M. P. et al. Tissue-specific reduction in splicing efficiency of IKBKAP due to the major mutation associated with familial dysautonomia. Am J Hum Genet 72, 749-758 (2003).
52 Lee, G. et al. Modelling pathogenesis and treatment of familial dysautonomia using patient-specific iPSCs. Nature 461, 402-406 (2009).

1. A method for transdifferentiation of a first somatic cell into a nociceptor cell, the method comprising increasing the protein expression of five nociceptor inducing factors selected from the group consisting of Asc11, Myt11, Ngn1, Isl2, Klf7, or a functional fragment thereof, wherein the nociceptor cell exhibits at least two characteristics of an endogenous nociceptor cell.

2. The method of claim 1, wherein a characteristic of the nociceptor cell is expression of at least two nociceptor specific genes selected from the group consisting of TrpA1, TrpV1, P2X3, NaV1.8, Prph and CGRP.

3. The method of claim 1, wherein the somatic cell is a fibroblast cell.

4. The method of claim 1, wherein the somatic cell is in vitro.

5. The method of claim 1, wherein the somatic cell is ex vivo.

6. The method of claim 1, wherein the somatic cell is a mammalian somatic cell.

7. The method of claim 6, wherein the mammalian somatic cell is a human somatic cell.

8. The method of claim 1, wherein the somatic cell is obtained from a subject.

9. The method of claim 8, wherein the subject is a human subject.

10. The method of claim 1, wherein the protein expression of a nociceptor inducing factor is increased by contacting the somatic cell with an agent which increases the expression of the nociceptor inducing factor.

11. The method of claim 10, wherein the agent is selected from the group consisting of: a nucleotide sequence, a protein, an aptamer, a small molecule, a ribosome, a RNAi agent, a peptide-nucleic acid (PNA), or analogues or variants thereof.

12. The method of claim 1, wherein protein expression is increased by introducing at least one nucleic acid sequence encoding nociceptor inducing factor protein selected from Asc11, Myt11, Ngn1, Isl2 or Klf7, or encoding a functional fragment thereof, in the somatic cell.

13. The method of claim 1, wherein the protein expression of Asc11 is increased by introducing a nucleic acid sequence encoding Asc11 polypeptide comprising SEQ ID NO: 1 or 11, a functional fragment thereof.

14. The method of claim 1, wherein the protein expression of Myt11 is increased by introducing a nucleic acid sequence encoding Myt11 polypeptide comprising SEQ ID NO: 3 or 13, a functional fragment thereof.

15. The method of claim 1, wherein the protein expression of Ngn1 is increased by introducing a nucleic acid sequence encoding
Ngn1 polypeptide comprising SEQ ID NO: 5 or 15, a functional fragment thereof.

16. The method of claim 1, wherein the protein expression of Isl2 is increased by introducing a nucleic acid sequence encoding Isl2 polypeptide comprising SEQ ID NO: 7 or 17, a functional fragment thereof.

17. The method of claim 1, wherein the protein expression of Klf7 is increased by introducing a nucleic acid sequence encoding Klf7 polypeptide comprising SEQ ID NO: 9 or 19, a functional fragment thereof.

18. The method of claim 11, wherein the nucleic acid sequence is in a vector.

19. The method of claim 18, wherein the vector is a viral vector or a non-viral vector.

20. The method of claim 19, wherein the viral vector comprises a genome which does not integrate into the somatic cell genome.

21. The method of claim 9, wherein the subject has, or is at risk of developing inflammatory and neuropathic pain.

22. The method of claim 9, wherein the subject has, or is at risk of developing a nociceptive pain related disease or disorder.

23. The method of claim 9, wherein the subject has, or is at risk of developing nociceptive pain.

24. The method of claim 22, wherein the nociceptive pain is pain accompanying a disease selected from the group consisting of rheumatoid arthritis, rheumatoid spondylitis, osteoarthritis, spondylosis deformans, gouty arthritis, juvenile arthritis, scapulohumeral periarthritis, fibromyalgia, and cervical syndrome; lumbago; lumbago accompanying spondylosis deformans; menalgia; pain and tumentia after inflammation, surgery or injury; pain after odontectomy; and cancer pain or pain or peripheral neuropathy on exposure to cancer chemotherapeutic agents.

25. An isolated population of nociceptors obtained from a population of somatic cells by a process of increasing the protein expression of five nociceptor inducing factors selected from the group consisting of Asc11, Myt11, Ngn1, Isl2 and Klf7, or a functional fragment thereof, in a somatic cell.

26. The isolated population of nociceptors of claim 25, wherein the nociceptor cell exhibits at least two characteristics of an endogenous nociceptor cell.

27. The isolated population of nociceptors of claim 25, wherein the somatic cell is a fibroblast.

28. The isolated population of nociceptors of claim 25, produced by the method of claim 1.

29. The isolated population of nociceptors of claim 25, wherein the somatic cell is a mammalian somatic cell.

30. The isolated population of nociceptors of claim 29, wherein the mammalian somatic cell is a human somatic cell.

31. The isolated population of nociceptors of claim 30, wherein the human somatic cell is obtained from a subject risk of developing a nociceptive pain related disease or disorder.

32. A method for treating a subject with nociceptive pain related disease or disorder, comprising administering a composition comprising an isolated population of nociceptors according to claim 25.

33. The method of claim 32, wherein the nociceptors are produced from a somatic cell obtained from the same subject as the composition is administered to.

34. An assay comprising an isolated population of nociceptors according to claim 25.

35. A kit comprising:
a nucleic acid sequence encoding a Asc11 polypeptide or a functional fragment thereof,
b. a nucleic acid sequence encoding a Myt11 polypeptide or a functional fragment thereof,
c. a nucleic acid sequence encoding a Ngn1 polypeptide or a functional fragment thereof,
d. a nucleic acid sequence encoding a Isl2 polypeptide or a functional fragment thereof, and
e. a nucleic acid sequence encoding a Klf7 polypeptide or a functional fragment thereof.

36. The kit of claim 35, further comprising instructions for direct transdifferentiation of a somatic cell into a nociceptor comprising at least two characteristics of an endogenous nociceptor cell.

37. A composition comprising at least one somatic cell and five nociceptor inducing factors selected from the group consisting of Asc11, Myt11, Ngn1, Isl2, Klf7, or a functional fragment thereof.

38. The composition of claim 37, wherein the somatic cell is a fibroblast cell.

39. A method for transdifferentiation of a first somatic cell into a nociceptor cell, the method comprising increasing the protein expression of one or more nociceptor inducing factors selected from the group consisting of Asc11, Myt11, Ngn1, Isl2, Klf7, Drgx, Ebf1, Etv1, Isl2, Pknox2, Brn3a, Runx1, Tlx3, or a functional fragment thereof, wherein the nociceptor cell exhibits at least two characteristics of an endogenous nociceptor cell.

40. The method of claim 39, wherein a characteristic of the nociceptor cell is expression of at least two nociceptor specific genes selected from the group consisting of TrpA1, TrpV1, P2X3, NaV1.8, Prph and CGRP.


SEQUENCE LISTING (20 sequences)

SEQ ID NO: 1 - protein, 236 aa, Homo sapiens
MESSAKMESGGAGQQPQPQPQQPFLPPAACFFATAAAAAAAAAAAAAQSAQQQQQQQQQQQQAPQLRPAADGQPSGGGHKSAPKQVKRQRSSSPELMRCKRRLNFSGFGYSLPQQQPAAVARRNERERNRVKLVNLGFATLREHVPNGAANKKMSKVETLRSAVEYIRALQQLLDEHDAVSAAFQAGVLSPTISPNYSNDLNSMAGSPVSSYSSDEGSYDPLSPEEQELLDFTNWF

SEQ ID NO: 2 - DNA, 2490 bp, Homo sapiens
agcactctctcacttctggccagggaacgtggaaggcgcaccgacagggatccggccagggagggcgagtgaaagaaggaaatcagaaaggaagggagttaacaaaataataaaaacagcctgagccacggctggagagaccgagacccggcgcaagagagcgcagccttagtaggagaggaacgcgagacgcggcagagcgcgttcagcactgacttttgctgctgcttctgcttttttttttcttagaaacaagaaggcgccagcggcagcctcacacgcgagcgccacgcgaggctcccgaagccaacccgcgaagggaggaggggagggaggaggaggcggcgtgcagggaggagaaaaagcattttcactttttttgctcccactctaagaagtctcccggggattttgtatatattttttaacttccgtcagggctcccgcttcatatttccttttctttccctctctgttcctgcacccaagttctctctgtgtccccctcgcgggccccgcacctcgcgtcccggatcgctctgattccgcgactccttggccgccgctgcgcatggaaagctctgccaagatggagagcggcggcgccggccagcagccccagccgcagccccagcagcccttcctgccgcccgcagcctgtttctttgccacggccgcagccgcggcggccgcagccgccgcagcggcagcgcagagcgcgcagcagcagcagcagcagcagcagcagcagcagcaggcgccgcagctgagaccggcggccgacggccagccctcagggggcggtcacaagtcagcgcccaagcaagtcaagcgacagcgctcgtcttcgcccgaactgatgcgctgcaaacgccggctcaacttcagcggctttggctacagcctgccgcagcagcagccggccgccgtggcgcgccgcaacgagcgcgagcgcaaccgcgtcaagttggtcaacctgggctttgccacccttcgggagcacgtccccaacggcgcggccaacaagaagatgagtaaggtggagacactgcgctcggcggtcgagtacatccgcgcgctgcagcagctgctggacgagcatgacgcggtgagcgccgccttccaggcaggcgtcctgtcgcccaccatctcccccaactactccaacgacttgaactccatggccggctcgccggtctcatcctactcgtcggacgagggctcttacgacccgctcagccccgaggagcaggagcttctcgacttcaccaactggttctgaggggctcggcctggtcaggccctggtgcgaatggactttggaagcagggtgatcgcacaacctgcatctttagtgctttcttgtcagtggcgttgggagggggagaaaaggaaaagaaaaaaaaaagaagaagaagaagaaaagagaagaagaaaaaaacgaaaacagtcaaccaaccccatcgccaactaagcgaggcatgcctgagagacatggctttcagaaaacgggaagcgctcagaacagtatctttgcactccaatcattcacggagatatgaagagcaactgggacctgagtcaatgcgcaaaatgcagcttgtgtgcaaaagcagtgggctcctggcagaagggagcagcacacgcgttatagtaactcccatcacctctaacacgcacagctgaaagttcttgctcgggtcccttcacctcctcgccctttcttaaagtgcagttcttagccctctagaaacgagttggtgtctttcgtctcagtagcccccaccccaataagctgtagacattggtttacagtgaaactatgctattctcagccctttgaaactctgcttctcctccagggcccgattcccaaaccccatggcttccctcacactgtcttttctaccattttcattatagaatgcttccaatcttttgtgaattttttattataaaaaatctatttgtatctatcctaaccagttcggggatatattaagatatttttgtacataagagagaaagagagagaaaaatttatagaagttttgtacaaatggtttaaaatgtgtatatcttgatactttaacatgtaatgctattacctctgcatattttagatgtgtagttcaccttacaactgcaattttccctatgtggttttgtaaagaactctcctcataggtgagatcaagaggccaccagttgtacttcagcaccaatgtgtcttactttatagaaatgttgttaatgtattaatgatgttattaaatactgttcaagaagaacaaagtttatgcagctactgtccaaactcaaagtggcagccagttggttttgataggttgccttttggagatttctattactgcctttttttttcttactgttttattacaaacttacaaaaatatgtataaccctgttttatacaaactagtttcgtaataaaactttttcctttttttaaaatgaaaataaaaaaaa

SEQ ID NO: 3 - protein, 1186 aa, Homo sapiens
MEVDTEEKRHRTRSKGVRVPVEPAIQELFSCPTPGCDGSGHVSGKYARHRSVYGCPLAKKRKTQDKQPQEPAPKRKPFAVKADSSSVDECDDSDGTEDMDEKEEDEGEEYSEDNDEPGDEDEEDEEGDREEEEEIEEEDEDDDEDGEDVEDEEEEEEEEEEEEEEEENEDHQMNCHNTRIMQDTEKDDNNNDEYDNYDELVAKSLLNLGKIAEDAAYRARTESEMNSNTSNSLEDDSDKNENLGRKSELSLDLDSDVVRETVDSLKLLAQGHGVVLSENMNDRNYADSMSQQDSRNMNYVMLGKPMNNGLMEKMVEESDEEVCLSSLECLRNQCFDLARKLSETNPQERNPQQNMNIRQHVRPEEDFPGRTPDRNYSDMLNLMRLEEQLSPRSRVFASCAKEDGCHERDDDTTSVNSDRSEEVFDMTKGNLTLLEKAIALETERAKAMREKMAMEAGRRDNMRSYEDQSPRQLPGEDRKPKSSDSHVKKPYYGKDPSRTEKKESKCPTPGCDGTGHVTGLYPHHRSLSGCPHKDRVPPEILAMHESVLKCPTPGCTGRGHVNSNRNSHRSLSGCPIAAAEKLAKAQEKHQSCDVSKSSQASDRVLRPMCFVKQLEIPQYGYRNNVPTTTPRSNLAKELEKYSKTSFEYNSYDNHTYGKRAIAPKVQTRDISPKGYDDAKRYCKDPSPSSSSTSSYAPSSSSNLSCGGGSSASSTCSKSSFDYTHDMEAAHMAATAILNLSTRCREMPQNLSTKPQDLCATRNPDMEVDENGTLDLSMNKQRPRDSCCPILTPLEPMSPQQQAVMNNRCFQLGEGDCWDLPVDYTKMKPRRIDEDESKDITPEDLDPFQEALEERRYPGEVTIPSPKPKYPQCKESKKDLITLSGCPLADKSIRSMLATSSQELKCPTPGCDGSGHITGNYASHRSLSGCPRAKKSGIRIAQSKEDKEDQEPIRCPVPGCDGQGHITGKYASHRSASGCPLAAKRQKDGYLNGSQFSWKSVKTEGMSCPTPGCDGSGHVSGSFLTHRSLSGCPRATSAMKKAKLSGEQMLTIKQRASNGIENDEEIKQLDEEIKELNESNSQMEADMIKLRTQITTMESNLKTIEEENKVIEQQNESLLHELANLSQSLIHSLANIQLPHMDPINEQNFDAYVTTLTEMYTNQDRYQSPENKALLENIKQAVRGIQV

SEQ ID NO: 4 - DNA, 7244 bp, Homo sapiens
gcgtgtgcatgggggtgtgtggaagtcaaacagatccctgaggcctgcgagcctatcgcgctgcatttgtcattcctgcccgggcgacattttttttttccctaagctactgcacactaaacagtgagagagcttttccctgcagtcttgttgaagcaccccgggttttttgctcattgttggtgggtgcattttaattttttcattccctggactatgggttatgatatccatactcactgaagacaaaaagccaccttttctgcgtcttggtggcatgcatgtgtctcatcatcctttcaaacttgtggtggaacagggttttcttccctgtctgtgtattttgagccagcacagttaccaaaattgaacttgtctttcgcttgtgagcggttgtggtcattgtgagggcgggtcatgaggaggctgtagccaaggacgaggtgtgtgcggctgttgcctggacgtttgtccaatccacgttgacatttgagggatcacagcgtgtgaaaatgaactcagaggagaattggtgaattcctatccagtgggcatcttcaaaccctggtcgacggcggaagaatatcaggtcctgagatcacccacccggcgcggcaacagtgcagagtggccacatctggtggaagaagaaaaaaatgtagttattgaattcaatcaagtgtttgcatctttcaagctatcaacaaaattccatcaagaaaggttccagttggtctcacagacgtatggatatccgaggagccacctaaagatggagaaatcaaggcatagagagattaagtgactttgccacagtcacaagctggagaggaccaggagtagagcttagagcgagcccctgactctgggcctgcgtcctgccaggagtcacgctgcctccgttcctaggagagaagacttcctgtaagatggaggtggacaccgaggagaagcggcatcgcacgcggtccaaaggggttcgagttcccgtggaaccagccatacaagagctgttcagctgtcccacccctggctgtgacggcagtggtcatgtcagtggcaaatatgcaagacacagaagtgtatatggttgtcccttggcgaaaaaaagaaaaacacaagataaacagccccaggaacctgctcctaaacgaaagccatttgccgtgaaagcagacagctcctcagtggatgagtgtgacgacagtgatgggactgaggacatggatgagaaggaggaggatgagggggaggagtactccgaggacaatgatgagccaggggatgaggacgaggaggacgaggagggggaccgggaggaggaggaggagatcgaggaggaggatgaggacgatgacgaggatggagaagatgtggaggatgaagaagaggaagaggaggaggaggaggaggaggaagaggaagaagaaaacgaagaccatcaaatgaattgtcacaatactcgaataatgcaagacacagaaaaggatgataacaataatgacgaatatgacaattacgatgaactggtggccaagtcattgttaaacctcggcaaaatcgctgaggatgcagcctaccgggccaggactgagtcagaaatgaacagcaatacctccaatagtctggaagacgatagtgacaaaaacgaaaacctgggtcggaaaagtgagttgagtttagacttagacagtgatgttgttagagaaacagtggactcccttaaactattagcccaaggacacggtgttgtgctctcagaaaacatgaatgacagaaattatgcagacagcatgtcgcagcaagacagtagaaatatgaattacgtcatgttggggaagcccatgaacaacggactcatggaaaagatggtggaggagagcgatgaggaggtgtgtctgagcagtctggagtgtttgaggaatcagtgcttcgacctggccaggaagctcagtgagaccaacccgcaggagaggaatccgcagcagaacatgaacatccgtcagcatgtccggccagaagaggacttccccggaaggacgccggacagaaactactcggacatgctgaacctcatgcggctggaggagcagttgagcccccggtcgagagtgtttgccagctgtgcgaaggaggatgggtgtcatgagcgggacgacgataccacctctgtgaactcggacaggtctgaagaggtgttcgacatgaccaaggggaacctgaccctgctggagaaagccatcgctttggaaacggaaagagcaaaggccatgagggagaagatggccatggaagctgggaggagggacaatatgaggtcatatgaggaccagtctccgagacaacttcccggggaggacagaaagcctaaatccagtgacagccatgtcaaaaagccatactatggtaaagatccctcaagaacagaaaagaaagagagcaagtgtccaacccccgggtgtgatggaaccggccacgtaactgggctgtacccacatcaccgcagcctgtccggatgcccgcacaaagatagggtccctccagaaatccttgccatgcatgaaagtgtcctcaagtgccccactccgggctgcacggggcgcgggcatgtcaacagcaacaggaactcccaccgaagcctctccggatgcccgatcgctgcagcagagaaactggccaaggcacaggaaaagcaccagagctgcgacgtgtccaagtccagccaggcctcggaccgcgtgctcaggccaatgtgctttgtgaagcagctggagattcctcagtatggctacagaaacaatgtccccacaactacgccgcgttccaacctggccaaggagctcgagaaatattccaagacctcgtttgaatacaacagttacgacaaccatacttatggcaagcgagccatagctcccaaggtgcaaaccagggatatatcccccaaaggatatgatgatgcgaagcggtactgcaaggaccccagccccagcagcagcagcaccagcagctacgcgcccagcagcagcagcaacctgagctgcggcgggggcagcagcgccagcagcacgtgcagcaagagcagcttcgactacacgcacgacatggaggcggcccacatggcggccaccgccatcctcaacctgtccacgcgctgccgcgagatgccgcagaacctgagcaccaagccgcaggacctgtgcgccacgcggaaccctgacatggaggtggatgagaacgggaccctggacctcagcatgaacaagcagaggccgcgggacagctgctgccccatcctgacccctctggagcccatgtccccccagcagcaggcagtgatgaacaaccggtgtttccagctgggcgagggcgactgctgggacttgcccgtagactacaccaaaatgaaaccccggaggatagacgaggacgagtccaaagacattactccagaagacttggacccattccaggaggctctagaagaaagacggtatcccggggaggtgaccatcccaagtcccaaacccaagtaccctcagtgcaaggagagcaaaaaggacttaataactctgtctggctgccccctggcggacaaaagcattcgaagtatgctggccaccagctcccaagaactcaagtgccccacgcctggctgtgatggttctggacatatcaccggcaattatgcttctcatcggagcctttcaggttgcccaagagcaaagaaaagtggtatcaggatagcacagagcaaagaagataaagaagatcaagaacccatcaggtgtccggtccccgggtgcgacggccagggccacatcactgggaagtacgcgtcccatcgcagcgcctccgggtgccccttggcggccaagaggcagaaagacgggtacctgaatggctcccagttctcctggaagtcggtcaagacggaaggcatgtcctgccccacgccaggatgcgacggctcaggccacgtcagcggcagcttcctcacacaccgcagcttgtcaggatgcccgagagccacgtcagcgatgaagaaggcaaagctttctggagagcagatgctgaccatcaaacagcgggccagcaacggtatagaaaatgatgaagaaatcaaacagttagatgaagaaatcaaggagctaaatgaatccaattcccagatggaagccgatatgattaaactcagaactcagattaccacgatggagagcaacctgaagaccatcgaagaggagaacaaagtgattgagcagcagaacgagtctctcctccacgagctggcgaacctgagccagtctctgatccacagcctggctaacatccagctgccgcacatggatccaatcaatgaacaaaattttgatgcttacgtgactactttgacggaaatgtatacaaatcaagatcgttatcagagtccagaaaataaagccctactggaaaatataaagcaggctgtgagaggaattcaggtctgaacagctgctgtagtgatgaaactcttgcttaaaaaggatgcctcttgttttttgctgctgtaacttaccagaaagtgttctatatttatttctgtttgaatttgaaacagtgttatgcttacaagacttcataatgattttatgtcttgctttaaagatagtacctgcagaatagtttttgaatacacccacattttgtacgtttccatgtaagctgacatagtgttctgccatgtaatgtttatagctgctgatgtatgcacatttgggggtatatctatttctgaagaggtaagctgatcaaaataaatagagtgtaaattctttttaatgctttagtgattaaatgttttagtattttgaactgaaatggacacacaaacacacacacgcacacacagacccacagctttgaatgatcatgttgtggctgagcagccgctttttagacgttatcattttgcctcatgttggaggactttatggaatttaagaaatacattttgtgtgcatattgtttcatagcaagaattcgttgcaaaaatgctttatttttgaacaatgcttggaaatattatgtgacttttttgtttgtttgttttaggaggatggtgtatggtgggggcaataaatgaggttttttgcattccaaggaaatggcatatggattaactgtaagaaatgaaataagtaatttattgtaagacaacatcaagccatggaaacttggcagaagattcaaagcagcttaaacagcacttttaaattaactcctaagcgttacatggttgtgactatggaaactccagttaagacaggatcttatcagaggtggacaacgtgaagatttccttttccattttcaataaactttggaacaaccttctcgtatctcccctagagtttcgtgcccctctgaactgtctgttattgcaatgtagtttatcaacagaatttgtgtgttttcgatttaagctaaaagataatttaagaacatttatttccccttttcactttaaaaaattatgattattcctattattgttatgaaccttcttattttacatttgagggataaaggcaaatgatttgtgagtcttctagttactggaccgagttttctgctggatctggtgggaaggcagctcggtaaagtttccctcctgctccccccgcccgactttgactctgaatcagcatttggtcctattcagaggactcttaccacgacgtttctgttctacacttgggtggagaccagttgaccatagagcatttgcagagcctcattgtttgatttcttgtgactattctaagaatgaatgcaatcagattttaaaagtaactaaatatacttcagcacttttttgctttaaactagatcatcttagacttgtttataccttccagatttgattgttttactcccaatgactgcactatatgtatgcataagaccacttttgagcgctgtgttcccccttctgagtagtcctttgacgacgtgttgtgttttctgatgttgacttgagttccatttagtagcatctcttccttccatgtcttgatgttatgcaggaagtacagacgtactttaaatttttgttatgaaataaaaaaaagatgggttttgtaaaaataaaaaaaaaatatttttagcagaacaggacttacagggtcattgtccccacaatgtgccagtcgactatttgcacttaccttgtcctatatatccgtacggaggtgtgcaattcctcgtgtcagtagccttgtgacactgaacctggatggattatagaggagccctcacggctgatcaataatgttgcaaagggagactacagggatctcacgacgaatattctgatacaatactcaacctcggtatatatatatgtgtataaatatatgtatatcccagcggcactttatactgttcactgtacaaaagcttacagttttccacaaggactttaataactagctgggaaaagacgatgtaattatttcggggctctgcggaaccttctctgtacagcgccccctttctgttgtgctattggttgcagctgccatgctcagaatgcgttttgagagctgaagcaaggtgcttgcagtcacctgaggccgtccgtgtggcccagggccccagctgcctttagggcccccattgttcataacagcatatgcatttccccaccgcgttgtgtctgcagcttctttgccaatatagtaatgcttttagtagagtactagatagtatcagttttggattcttattgttatcacctatgtacaatggaaagggattttaagcacaaacctgctgctcatctaacgttggtacataatctcaaatcaaaagttatctgtgactattatatagggatcacaaaagtgtcacatattagaatgctgacctttcatatggattattgtgagtcatcagagtttattataacttattgttcatattcatttctaagttaatttaagtaatcatttattaagacagaattttgtataaactatttattgtgctctctgtggaactgaagtttgatttatttttgtactacacggcatgggtttgttgacactttaattttgctataaatgtgtggaatcacaagttgctgtgatacttcatttttaaattgtgaactttgtacaaattttgtcatgctggatgttaacacatcttactctaaataaacaaggtgttgccacatttgtagcacgaaggatctctaa

SEQ ID NO: 5 - protein, 237 aa, Homo sapiens
MPARLETCISDLDCASSSGSDLSGFLTDEEDCARLQQAASASGPPAPARRGAPNISRASEVPGAQDDEQERRRRRGRTRVRSEALLHSLRRSRRVKANDRERNRMHNLNAALDALRSVLPSFPDDTKLTKIETLRFAYNYIWALAETLRLADQGLPGGGARERLLPPQCVPCLPGPPSPASDAESWGSGAAAASPLSDPSSPAASEDFTYRPGDPVFSFPSLPKDLLHTTPCFIPYH

SEQ ID NO: 6 - DNA, 1717 bp, Homo sapiens
cacacgagctgatctgatcgccggcgacatcactcaggagaccggccgggcgcgtggcccctgcaggcgaggcgaggaggccaggccaagttctccgtgcgcccctgcacccttccaggctctcgcacccgcaactggcacagagtaacaaccccaggctgttgggaacgtaagtgcgccctggcggctctgccctcagtccgggctgcagcgctctgagcgcctttctatctgtccgtcggtcctgcacagcgcaacgatgccagcccgccttgagacctgcatctccgacctcgactgcgccagcagcagcggcagtgacctatccggcttcctcaccgacgaggaagactgtgccagactccaacaggcagcctccgcttcggggccgcccgcgccggcccgcaggggcgcgcccaatatctcccgggcgtctgaggttccaggggcacaggacgacgagcaggagaggcggcggcgccgcggccggacgcgggtccgctccgaggcgctgctgcactcgctgcgcaggagccggcgcgtcaaggccaacgatcgcgagcgcaaccgcatgcacaacttgaacgcggccctggacgcactgcgcagcgtgctgccctcgttccccgacgacaccaagctcaccaaaatcgagacgctgcgcttcgcctacaactacatctgggctctggccgagacactgcgcctggcggatcaagggctgcccggaggcggtgcccgggagcgcctcctgccgccgcagtgcgtcccctgcctgcccggtcccccaagccccgccagcgacgcggagtcctggggctcaggtgccgccgccgcctccccgctctctgaccccagtagcccagccgcctccgaagacttcacctaccgccccggcgaccctgttttctccttcccaagcctgcccaaagacttgctccacacaacgccctgtttcattccttaccactaggccctttgtagacactgttactttccccctcccctagtcagcaggcaatagatggggccccagctgccgcctcgggaccccctctccaggcggagggaggaagcgggagctttaaagcagtcggggatacctgagccgcttgttaggtcgccgcaccctcgcggcggatgtctcttggtctgtttctccggccctcagcccagcgcccctcctgcccgcccctagacggcctttccttttgcactttctgaactccacaaaacctcctttgtgactggctcagaactgaccccagccaccacttcagtgtgatttagaaaagggacagatcagcccctgaagacgaggtgaaaagtcaattttacaatttgtagaactctaatgaagaaaaacgagcatgaaaattcggtttgagccggctgacaatacaatgaaaaggcttaaaaagcagagacaaggagtgggcttcatgcattatggatcccgacccccaccactgcagactcgctctaggaagaactggagactcttgcttagctattcaggcacagggctggagagtactttaatttattcaagatgcttcattcatatgaaaatgtatttttgtacataaagagtttattctattatgagctatcaaagtttacatttttgtactgcagacgcttcatgtaaataaaaactaaaaataaaaaaaaaaaaaaaaaaaaaaaaaaaaaaaaaaaaaaaaaaaaaaaaaaa

SEQ ID NO: 7 - protein, 359 aa, Homo sapiens
MVDIIFHYPFLGAMGDHSKKKPGTAMCVGCGSQIHDQFILRVSPDLEWHAACLKCAECSQYLDETCTCFVRDGKTYCKRDYVRLFGIKCAKCQVGFSSSDLVMRARDSVYHIECFRCSVCSRQLLPGDEFSLREHELLCRADHGLLLERAAAGSPRSPGPLPGARGLHLPDAGSGRQPALRPHVHKQTEKTTRVRTVLNEKQLHTLRTCYAANPRPDALMKEQLVEMTGLSPRVIRVWFQNKRCKDKKKSILMKQLQQQQHSDKTSLQGLTGTPLVAGSPIRHENAVQGSAVEVQTYQPPWKALSEFALQSDLDQPAFQQLVSFSESGSLGNSSGSDVTSLSSQLPDTPNSMVPSPVET

SEQ ID NO: 8 - DNA, 1836 bp, Homo sapiens
gcaaagagccgaggccgggcgcgcgaccctcgtccttctgcccctggccgcacactttgcgcacatctctttttctgcatggtggatattatttttcattatccttttctgggtgctatgggtgatcattccaagaagaagcccgggacggccatgtgcgtgggctgcgggagtcagatccacgaccagtttatcctgcgggtgtcgcccgacctcgagtggcacgcggcctgcctcaagtgtgccgagtgcagccagtacctggacgagacgtgcacgtgcttcgtgagagacgggaagacctactgcaagcgggactacgtcaggctgttcggcatcaagtgcgccaagtgccaggtgggcttcagcagcagcgacctggtgatgagggcgcgggacagcgtgtaccacatcgagtgcttccgctgctccgtgtgcagccgccagctgctgcctggggacgagttctcgctgcgggagcacgagctgctctgccgcgccgaccacggcctcctgctcgagcgcgccgcggccggcagcccgcgcagccccggcccgcttcccggcgcccgcggcctgcatctgcccgacgctgggtcgggccggcagcccgcgttgcgcccgcacgtgcacaagcagacggagaagacgacccgcgtgcggactgtgctgaacgagaagcagctgcacactctgcggacctgctacgccgccaacccgcggcccgacgctctcatgaaggagcagctggtggagatgaccggcctgagcccgcgggtcatccgcgtctggttccagaacaagcgctgcaaggacaagaagaaatccattctcatgaagcagctgcagcagcagcagcacagcgacaagacgagccttcagggactgactgggacgcccctggtggcgggcagtcccatccgccatgagaacgccgtgcagggcagcgcagtggaggtgcagacgtaccagccgccgtggaaggcgctcagcgagtttgccctccagagcgacctggaccaacccgccttccaacagctggtctccttctccgagtccggctccctaggcaactcctccggcagcgacgtgacctccctgtcctcgcagctcccggacacccccaacagtatggtgccgagtcccgtggagacgtgagggggacccctccctgccagcccgcggacctcgcatgctccctgcatgagactcacccatgctcaggccattccagttccgaaagctctctcgccttcgtaattattctattgttatttatgagagagtaccgagagacacggtctggacagcccaaggcgccaggatgcaacctgctttcaccagactgcagacccctgctccgaggactcttagtttttcaaaaccagaatctgggacttaccagggttagctctgccctctcctctcctctctacgtggccgccgctctgtctctccacgccccacctgtgtccccatctcggccggcccggagctcgcccacgcggacccccgccctgccccagctcagcgctccctggcggcttcgcccgggctcctagcggggaaaaggaaggggataactcagaggaacagacactcaaactcccaaagcgcatgattgctgggaaacagtagaaaccagacttgccttgaaagtgtttaagttattcgacggaggacagagtatgtgagcctttgccgaacaaacaaacgtaagttattgttatttattgtgagaacagccagttcatagtgggacttgtattttgatcttaataaaaaataataacccgggaaaaaaaaaaaaaaaaaaaaaaaaaaaaaaaaaaaaaa

SEQ ID NO: 9 - protein, 302 aa, Homo sapiens
MDVLASYSIFQELQLVHDTGYFSALPSLEETWQQTCLELERYLQTEPRRISETFGEDLDCFLHASPPPCIEESFRRLDPLLLPVEAAICEKSSAVDILLSRDKLLSETCLSLQPASSSLDSYTAVNQAQLNAVTSLTPPSSPELSRHLVKTSQTLSAVDGTVTLKLVAKKAALSSVKVGGVATAAAAVTAAGAVKSGQSDSDQGGLGAEACPENKKRVHRCQFNGCRKVYTKSSHLKAHQRTHTGEKPYKCSWEGCEWRFARSDELTRHYRKHTGAKPFKCNHCDRCFSRSDHLALHMKRHI

SEQ ID NO: 10 - DNA, 8433 bp, Homo sapiens
agagaagcgatcgcgagagaaaaaaatgcaacctcccaaaataaagagcaaagattgcattaggagcgaacagcgctgcagaaatagatggcagcttcgtgtcagtgagtttgcatcccccttcctgatccacgagctggagtgattagagccctggaagggaattgttactcccgtggagaagtccccttttcctggcagtcgtctgcactgtacacgctggatgcctctctccatccaccccactcactcgctcctctctcacctcctctctccctctcctgcattgatttttttttttcctttttagttgactgaaacaaaacaaaacaaaagggccactggatgtctgccttcttggggggtgagccagacagactgacaaacaaacagccccaactgtgttcgggggagggtttcgcctcccgttttgcccggcagcagcagcatggacgtgttggctagttatagtatattccaggagctacaacttgtccacgacaccggctacttctcagctttaccatccctggaggagacctggcagcagacatgccttgaattggaacgctacctacagacggagccccggaggatctcagagacctttggtgaggacttggactgtttcctccacgcttcccctcccccgtgcattgaggaaagcttccgtcgcttagaccccctgctgctccccgtggaagcggccatctgtgagaagagctcggcagtggacatcttgctctctcgggacaagttgctatctgagacctgcctcagcctccagccggccagctcttctctagacagctacacagccgtcaaccaggcccagctcaacgcagtgacctcattaacgcccccatcgtcccctgagctcagccgccatctggtcaaaacctcacaaactctctctgccgtggatggcacggtgacgttgaaactggtggccaagaaggctgctctcagctccgtaaaggtgggaggggtcgcaacagctgcagcagccgtgacggctgcgggggccgttaagagtggacagagcgacagtgaccaaggagggctaggggctgaagcatgtcccgaaaacaagaagagggttcaccgctgtcagtttaacgggtgccggaaagtttatacaaaaagctcccacttaaaggcccaccagaggactcacacaggtgagaagccttataagtgctcatgggagggatgtgagtggcgttttgcacgaagcgatgagctcacgaggcactacaggaaacacacaggtgcaaagcccttcaaatgcaaccactgcgacaggtgtttttccaggtctgaccatcttgccctccacatgaagagacatatctaaaaaaccgaaaggccagagttgccatggcatcggctagtgtctaaaggaaacgccatgaggcagggggctggacttcaggcggggacccattgcctcgcagaagaaagttctcacttataaacctctgtacacacacacacacacacacacatatacacacactcacagacccacacacatacacactgtcatgcactcaactatatttaaaatatatacgtctattctttatgccttgccctagccagatggaagaagatgaagaaggaaaccaggtgaactcagcaaggcagactggctgcttacttcagcactattggaattatttcccgctgttgccaatggaaatcaaagaaaatggatgtgacgtctgtgcaggtggacggcagtccgaggggcttatttcacttgcttctcagtgcaacttgataggagaatccagcatcttaaagttgcatatgtgtagcactaatgtttctttttaaatagttgggggaaaatgacctagaaaaccaaattgcagtttggtagccaaaattaactcttggtttatttgtcctttgtgtgtgaaaagtcctactattccgtgcgtcagacttcctcacagaactgttgactggttttggttcttagtactattgagatctttcgcgtcgatcccaacggccttagcggcggcagactggaataacaccttacacctttctggcctgcatttctgtagacttcactctcaagggaggagttttcttttcttacgttttgacttttgcacaccatatgcactagggattctggaaacttctagcatgactgcaaagtggccaagagaataaagtccttgatgataaatcacagtatatcccttgagcctcaccttattgccagtgctagattttttctttttaatctctccgtttttgctaacgaaaacttgaaaagcttatttggaagcttaaatgttttatcttttctccatggactaaacctctccaggactctctcggcacctggatgtccagctctcgaagcagccagtcagatgggacatcacagttctctcatcctccttgaggcatgatgacctcagctcatagtgatcaaccgttgtgctgtgtgtcattgctaccccataaccagttacagcatagatgtcgctagtctcagagggcagctgcgtatttaatttaactctggtttatgacctgacaaaaagccaaaaatatcactctttccaggagtggggaaaactgaggatgcctcccaagtctagtggcttcacaaaagatcatcctgtcttctctgtcatgcccactgagctcctattcccctacgtgttacaatacacaatttaaaacgccattgtgggagtgaagggttgacatttaaggaaaaggttgaggtgtttctctcatgggctgtctaaaaggagagacacgtttctttctttccttttttttttggctaggcccaccatgacttgtgacctagaacccccaggattaacagaggcctcacatttactctgcaagctgactccaaaggagtctacagtccttacttgtcatgccacactcacacatccagtagtggtctctatctacccgcattcctagctagctggcactggcctcaactccaaagactgcctttaggaccatcaaatggcctatgcaagcaagcggggtggttattaggacagattgtatattttgtatattctgggaccatcccttcaagacacgtctataaaacaaaaatggcgcttggtccacacacggttgctgctccctcctaccagctggctcccctcctgtcctcctttgactgtttgactcattgactgttaaaatgccaccccatacatatttgggatgcaaaactgaagtcaaaaggaaataatataagaaacacaaacacatatatgacagcaaccttcaagatctgggttttcagctttctgcaacctttgttttcactgaaatgttgaaactactcgtctgagggcaaaggaacctcctcacaaatgctatagctgccaattggacacttggggcatttcgaggtctggccctaagaatttactttctccttttccttttttctatttagaccaaaaaaaacaaaaacaaaaacaaaaaaaaaaacaaaataatacaaaacgaaaaaaaaagaaagaacacccgttaacacacacgcgcacacacacacaaaatctgtccatttgccggaggcaattgtatgtatgttagttggagggtattaaaaatcagttttattccaaagatttaaaactagacatgacttaaaaacaatttctggagcactgcttgctgacaatctcgtagttctctgctgcatttgagtgcattttgtggccagtccatcagggcgtaccatgggattatatttgaatgtgtggtgcatccttcctggatgaaggatgtgtgagggaccttgaacctcagctgtattaaactgtagcgcctccagtcagtgcactagatgaaacttttagacaccctgaattctgttggttcctttcttttcctttatgtagcagcctccagcatgaatgcacgcacacgccagtgatggcattaagccatggccaccacgatttgcaaatgttctctcccaagctggagctgctcttgcctctcgaatgctattattaagggtttataatacttaatttaattttcgaactgaccaatgcaaggctctattaaaaagaaagtttaaaaaatgcaaaagagtaatcattgcttgtttgctccctattttcatctgtggtctcatttgaatgtggcagaacaaaggccctttggtcctcatcagtgtctgaaatgttcagtaatttctctctcttttgtatcagtgaggtcctttgtaatctgctcctgacctttcttggagcagggtgcattgaaactcaatggtggtgcttgcttgcttcagagtcatttgttgactgtgagaattggcctaagaatttggtgggtgctaagtggatggctttgaaactgttcttctttagccgagttgacacctgtgaatgatgaccagtcctgatcattttggaaatggatttgtaataaaacgtccatcacctctgcagtggcagagatggttactaagagccgctagagcgagcaggttttccaagaagtaacctgaagacattttgctcccaagaggactggttatttaaaacagtgcattaatggacatttgaaacacattaaacccctttctcatttcagttgttacctcctaaccctccaggggatcccaaatttgaaaggaaaaacccggcctggtgtttctggtggtgtcctaacaagcacgcttttatccagggttcagatttgttcatgtagaaaaagagtttctaagccactgacaattttttttttttgtaatttcaaattatacttctttctcctgccacatgactgtaagtcatagacatggaaacctgaaattataatgctgctcctagctactggcctcctgccccacccatggttaatggctcagctcaatgcctggtggtaatgagtattatgtccagaaaaagagatgttcagattccatgacaaagctgcatttttgtaaaaatattggagaccccaaaatgaacttcatgctgaccatttcctcctctctgtgtgctttcccttgcaaagcccttcaaatatcctcttctctcgacgccatctcctctccacctgcacctcttgtgccctttgtacatctttgattgcctgatgataacagggtaaaaggacagccaacctcatgcctgattagcagaactgaatcctagttttaaaaaatcttctctggcttcagagaagattttataaggacttttgtttgggataagctttccagattatccatgtctatttgcatcaaaggggaaagaaatggggcttttggatggctcttccagtgcattcggaacattgcctcttgcctttattcctgcattttatggcaaagccaaaagaaactcaagttgcaagaacaaaacccagtgactcgttttgatggttcaaaatggtttcctttatggaagtcacttcataaaatgttaagtaaaaagtgggaagtgcttctgtcttctcttttgcatgagttgcttttaggagcaggaagaaggtaggcaaagtaagataaagatgcaacacatttaactacaaaaatcaggttcattttttagtttattagaatttttttgaaatcttaagagggccagcatttctggctacaattttgcacccagaacattgccaaaatgaacattcagtaaatagaacctgattgaaatttactcctggaagctttcctttgcattttcgggaagtggccacctgccaagcgcaagagttggggggcaggaggggaggactcaaattcagggtgtctggattaaatttcggtgaacatggtgatatctcagtttgaaaactagagggcctatcctgagtatacatcaatgtctctttgatggcctactttcctcagtgaggatctttgggaatacttgagatggaacaacagaaatgtgtgaaaggaagcagaaacttcttgtaaataacgtgacctcccacgacgaactgcctgaggcttcagggttttttcttgcttttaacactcttaaatctcctctgttggttcctaatagatcccagaaaagggaaaaataaagctgcagttaactttcttatgtgcatccttccaatagagtactgtatttttcaggtgttttgcatttaacataaaagtcctcgggaaacaggtgtcaaaaacagagagagaaatcctgggccatcacttcacaaatatcccaaacaagatattcttttcaaacagggctccctctcagtggtcatgagggaaggttgataatgttctttgttggggactgtttatacaattttttttcaactgtgagctttggaatcgtaacttgctgtgagtccagcttctgtctactgccataagatggaccccacgtcagcataatgagggtggtatatatgctcgcacctagacatgcgcatatgtacctgtcgtaccttcacggaaggaaaacaggctactgacgtttcggaggagtagccaccagtgcctaatatcttttgggggggatggatgcttataattgccagtatatcgaaaccacactgggagttccacatagcggggaggggttgggggtgggcagaggggacattttaaacctaggcctttggactggaggcagaacgatttctgcaaacctaggtcctgaaggctttggggcttattggctggttctcaacctttttgttttttcttcccagcatgcatttcctatctaaacccagacttagtttaatttccttatctttcacttctgcttcattccagggaggaaaaatacacctgttaatggccaagatctccttgctaacacagaggcaaaaataaatgtctaatgtttttgaagcctccccttcctttccacaagcccccacccgcccccgcgtcaagctccttctcccacttcctactcccacacaacttcccagccactgaaacttttctttcaaatctctattatcctcttaacagttgcttgaataaatttatttttgcactatacattttctttttgccagatgtgtctaacaagtgtgtttggagagacctactcccagccccgtctccttccccgcctccccccgtcacattctctcaggccttctctggtatttataatatatcacagaagtacccagtcttatagccctcggttatgcctttttttgacattttattttttttaagctttttatatatatatatatataaatatattactttgtcaagtttttttgctgtacaaaagtcttaagatttaaaactattatttgtattatatgatggtggtatgttaatgttacaaaattattaatgaagaaaaaatttatttttgttactggtctgtttcataattcttttttaaattggtatattgtaagatatctatgcaaaaaatgttatgtgacgcatttttatttaagaatgtaatatgtgtaataaacagtagaatgtgtttggccttggaatactttactgtatttctccttagcttgtttcactggggaaaaaaatcttcgaaagacgcaagtgggtacttacatacttcgtgaaagttttctttcttggagaaagggaaagcaaaaggttgtattaggttatcttcgtttgggaagttgtgtgtgtgtgtgcgtatgtgtgtattttatagtttcattgaggcagctcaatgcccaaataagggtcactgagtttatttcttcaaggggaaaaagggagccaattgttggagattatgaaaagcaatattttagaatgatagagattacaagatgttatttgtttagggggttgggagaggcttattgaaagcggtttatttggcgagagaaggaggcagtttgttctgggatggtgtttaataggaacctattgggaaaggatctttgaagcagtctgtgaaggaggaagggtaaagatcagagggaataatttaggtgaggggtagggggcagtaaaatgacaggaggtggttggagtggggaggaaatgggtaaccggaagccaggaaatccagctggctgtggagagtacaaaaaactagatggaaatacaagcagcttcagacccagagaagagagggagatgaaagccccagggaaaattctcagaactgaaaagaaaagtactaaaatctctgccacacacgacttccaggaaagagcatcaccagtaaggaggaaggtagagaacccagctggtggtgtcgcctcagcatcccgagctcagcgattccccgagagaagtggtgtcattcacaggaaacagcagtaaaacacatttgtcacatgggacacagcagtagtcaagctttctttgcattctttggacttacagaagtggatacggtggtgaataacctctatccctaatcaaatgaatctgacaagaaactttccaataaatgtttactttagaaa

SEQ ID NO: 11 - protein, 231 aa, Murine spp.
MESSGKMESGAGQQPQPPQPFLPPAACFFATAAAAAAAAAAAAQSAQQQQPQAPPQQAPQLSPVADSQPSGGGHKSAAKQVKRQRSSSPELMRCKRRLNFSGFGYSLPQQQPAAVARRNERERNRVKLVNLGFATLREHVPNGAANKKMSKVETLRSAVEYIRALQQLLDEHDAVSAAFQAGVLSPTISPNYSNDLNSMAGSPVSSYSSDEGSYDPLSPEEQELLDFTNWF

SEQ ID NO: 12 - DNA, 2259 bp, Murine spp.
agcagtctctcacttctggccagggaacgtggaaggcgtaccggctgggagccggttagggagggcgaattggggggaacgagagagcaattagaaagaaagggggttcaaccaaataatcccagaagcaggctcaagcccaggctggagcaagggagagcgggcgcaagaaagcgcagccccggagcagctccacctggcagagtgcgctcggcactgacttttgcggctgctttccttttccctttcctcttttaaaaccgagaaggcgccggcggcggccgcacacgcgagcgccacgcgaggctcccgaagccaaccgcggcgggaggaggggagggaggaggcggcgcagagggaagacgatcgcccaggcaccttcctccgctgcagcctgacaactctgcctccttctgcgcgtttcttcccctttaactttcctccggggctcgtttctcccctctcctttttcttcgtccccctttgatcgtgcttcgcagccccgcttccttcaagggctctgcgcaccctgcgtccccaactcgttctcccccgcgacagtttggcccggcatggagagctctggcaagatggagagtggagccggccagcagccgcagcccccgcagcccttcctgcctcccgcagcctgcttctttgcgaccgcggcggcggcggcagcggcggcggccgcggcagctcagagcgcgcagcagcaacagccgcaggcgccgccgcagcaggcgccgcagctgagcccggtggccgacagccagccctcagggggcggtcacaagtcagcggccaagcaggtcaagcgccagcgctcgtcctctccggaactgatgcgctgcaaacgccggctcaacttcagcggcttcggctacagcctgccacagcagcagccggccgccgtggcgcgccgcaacgagcgcgagcgcaaccgggtcaagttggtcaacctgggttttgccaccctccgggagcatgtccccaacggcgcggccaacaagaagatgagcaaggtggagacgctgcgctcggcggtcgagtacatccgcgcgctgcagcagctgctggacgagcacgacgcggtgagcgctgcctttcaggcgggcgtcctgtcgcccaccatctcccccaactactccaacgacttgaactctatggcgggttctccggtctcgtcctactcctccgacgagggatcctacgaccctcttagcccagaggaacaagagctgctggactttaccaactggttctgaggacctgccaggctctcctgggaatggactttggaagcaggatggcagcagatcctgcatctttagtgtttctcgccaacgacgtcaaatggggaggcagaaaaacaaggggaaaaaagaagaagaaatgaaacaaacaaaccagacagccaacctacaggggcaccttcactaagatgcaatgttctcagcaaacaggggtgggctccaacagtgtctctgcattccaacatcatttccagacacgagaagagtgactggtgtctgaacctaagcccgaatcacagatgggttcctttcctggagcaagagcgtcacacacacacacacacacacagacagacactatattaactcccaaccactaacaggcagggctggaagcgcgcatgtgcaagtgccttcacctcccactctctgtcagagctgtcttagccccctgaaactgggttgatgtctttcctcagtcacccccattccagcgatctatggacatttgcctccattgaagcaacgtcagttctcggacagcctttccctctcctggtggcctcctccccaaaccccacatcgccctcccacggtctttgcttctgttttcttcatagaatgcttccaatctttgtgaatttttttattataagaaaaaaatctatttgtatctatcctaaccagtttggggatatattaagatatttttgtacataagaaaaagagagagaaaaaatttatagaagttttgtacaaatggtttaaaaatgtgtatatcttgatactttaacatgtaatgagattacctctgcgtactttagatatgtagttcatcttacaactgccatccccacccccatccccagtgtggttttggaaagaactctcctcataggtgagatctaaatgccaccagaatgacttcagcaccaatgtgtcttacttcacagaaacgtggttaatgtattaatgatgttattaaaaaaaactgttcaagaag

SEQ ID NO: 13 - protein, 1187 aa, Murine spp.
MDVDSEEKRHRTRSKGVRVPVEPAIQELFSCPTPGCDGSGHVSGKYARHRSVYGCPLAKKRKTQDKQPQEPAPKRKPFAVKADSSSVDECYESDGTEDMDDKEEDDDEEFSEDNDEQGDDDDEDEVDREDEEEIEEEDDEEDDDDEDGDDVEEEEEDDDEEEEEEEEEEENEDHQMSCTRIMQDTDKDDNNNDEYDNYDELVAKSLLNLGKIAEDAAYRARTESEMNSNTSNSLEDDSDKNENLGRKSELSLDLDSDVVRETVDSLKLLAQGHGVVLSENISDRSYAEGMSQQDSRNMNYVMLGKPMNNGLMEKMVEESDEEVCLSSLECLRNQCFDLARKLSETNPQDRSQPPNMSVRQHVRQEDDFPGRTPDRSYSDMMNLMRLEEQLSPRSRTFSSCAKEDGCHERDDDTTSVNSDRSEEVFDMTKGNLTLLEKAIALETERAKAMREKMAMDAGRRDNLRSYEDQSPRQLAGEDRKSKSSDSHVKKPYYGKDPSRTEKRESKCPTPGCDGTGHVTGLYPHHRSLSGCPHKDRVPPEILAMHENVLKCPTPGCTGRGHVNSNRNSHRSLSGCPIAAAEKLAKAQEKHQSCDVSKSNQASDRVLRPMCFVKQLEIPQYGYRNNVPTTTPRSNLAKELEKYSKTSFEYNSYDNHTYGKRAIAPKVQTRDISPKGYDDAKRYCKNASPSSSTTSSYAPSSSSNLSCGGGSSASSTCSKSSFDYTHDMEAAHMAATAILNLSTRCREMPQNLSTKPQDLCTARNPDMEVDENGTLDLSMNKQRPRDSCCPVLTPLEPMSPQQQAVMSSRCFQLSEGDCWDLPVDYTKMKPRRVDEDEPKEITPEDLDPFQEALEERRYPGEVTIPSPKPKYPQCKESKKDLITLSGCPLADKSIRSMLATSSQELKCPTPGCDGSGHITGNYASHRSLSGCPRAKKSGIRIAQSKEDKEDQEPIRCPVPGCDGQGHITGKYASHRSASGCPLAAKRQKDGYLNGSQFSWKSVKTEGMSCPTPGCDGSGHVSGSFLTHRSLSGCPRATSAMKKAKLSGEQMLTIKQRASNGIENDEEIKQLDEEIKELNESNSQMEADMIKLRTQITTMESNLKTIEEENKVIEQQNESLLHELANLSQSLIHSLANIQLPHMDPINEQNFDAYVTTLTEMYTNQDRYQSPENKALLENIKQAVRGIQV

SEQ ID NO: 14 - DNA, 7198 bp, Murine spp.
gtggagctgcgtgcattaggggctggctgtggaggtaaagcagatctcttaggcccgctagcctctcctgctgcatgtgtcattcctgcccgcgcgacattttccccctaagctactgcacacaaaacagagcgagaaagcttctccctgcagtcttcttggaggcctcctggtttctcacccattgttggtgggtgtatttcaattttttgattccctggactgtgggttatgaaatcaatgctgctgaagacaaaagcaaccttccctgcctctcggtgctatgcgtggtcctcctcggcctcccacttgtgggggaaagggttttctctttctttctgtgtgttttgagccagcacagttaccaaaattgaacttgccgtcacttgtgagcggtgtggtcatggtgtgaggggtcccacagaggctgcagctgaggtctgggtgtgtgcaattctcagctgggctttgccctacccaggttgacgactgaagaatcacagagtgtggaaaagaacacaaggagaaattggtgagaacatctgcctagcatctccaagtcctgtggagggagccagcagtgctggtccaaagaggacccagagagtgaactcagagtgaccacatctgatagaagaggggaagatgtagtttctgagtccagtccagtgtttgtgtctctcacattgtcaacaaaagaaaggctccagctgtccccacagacatatggatattccaggagccacgtaaagatggagaaatggaggcacagagaaattaagtgacttggccacagtcacaagctggggaggaccagggaaagcctagagagagctggctctgggcctgcatcctgcccacggagtcaccctgcctccgtcctcaggagagaaggcttcctacaagatggacgtggactctgaggagaagcgccatcgcacacggtccaaaggggttcgagttcctgtggagccagccatacaagagctgttcagctgtcccactccaggctgcgacggcagtggtcacgtcagtggcaaatatgcacgacacagaagtgtatatggttgtcccttggctaaaaaaagaaaaacgcaagataaacagccccaagaacctgctcccaagcgaaaaccatttgcagtaaaagcagatagttcctcagtagacgaatgttatgagagtgatggtactgaagacatggatgataaggaggaagatgatgatgaggagttctctgaagacaatgatgagcaaggggatgatgacgacgaagatgaggtggatcgggaagacgaggaggagatcgaggaggaagatgatgaagaagatgatgatgatgaagatggtgacgatgtagaagaggaagaagaggatgatgatgaagaggaggaagaagaggaagaggaagaagaaaatgaagaccatcaaatgagttgtactcgaataatgcaggacacagacaaggatgataacaacaatgatgagtatgataactatgatgaactggtagctaagtcgctattaaatcttggcaaaattgctgaggatgcagcataccgagccaggactgaatcagagatgaacagcaatacctccaatagtctggaggacgatagtgacaaaaacgaaaacctcggtcggaaaagcgaactgagtctagacttagacagtgatgttgttagagaaacagtggactcccttaagctgttagcacaaggacatggtgttgtgctatcagagaatatcagtgacagaagttatgctgaggggatgtcacagcaggacagtagaaatatgaactatgtcatgctagggaagcccatgaacaatggactcatggagaagatggtggaggagagtgatgaggaagtgtgtctaagtagtctagagtgcctgaggaaccagtgctttgacctggccaggaaactcagcgagaccaacccacaggacaggagtcagccacccaacatgagtgtgcgccaacatgtccggcaagaggacgacttccctgggaggacgccagacaggagctactcggatatgatgaaccttatgcggctggaggagcagctcagtcccaggtctagaacgttctccagctgtgccaaggaggatgggtgtcatgagagggatgatgacaccacctcagtgaactcagacaggtctgaggaagtgtttgacatgaccaagggcaacctgactctgctagagaaagccattgccttggagacagagagagccaaggccatgcgggagaagatggccatggatgctgggagaagggataacctgagatcctatgaggaccagtctccaagacagctggctggggaagacagaaaatccaaatccagtgacagccatgtcaaaaagccatactatggtaaagatccctcaagaacagaaaagagagagagcaagtgtccaacccccgggtgtgatggaaccggccacgtaactgggctttacccgcatcaccgcagtctgtctggatgcccgcacaaagatagggtccctccagaaattcttgccatgcatgaaaatgttctcaagtgtcccactccaggctgcacagggcgagggcatgtgaatagcaacaggaactcgcacagaagcctctctggatgccccattgctgctgcagaaaaactggcaaaggcccaagagaaacaccagagctgtgatgtgtccaaatccaaccaggcctcagaccgagtcctcaggccaatgtgctttgtcaaacagcttgagattcctcagtatggctacagaaacaatgttcccacaaccacaccacgctccaacctggccaaggagcttgagaaatactccaagacttcgtttgagtacaacagttacgacaaccatacttatggcaaaagagccatagctcccaaggtgcaaaccagggacatatcccccaaaggatatgacgatgccaagcggtactgcaagaatgccagccccagcagcagcaccaccagcagctatgcacctagcagcagcagcaacctcagctgtggtggtggcagcagcgccagtagcacgtgtagcaagagcagctttgactacacacatgacatggaggccgcacacatggcagccacagccattctcaacctgtccacacgttgtcgtgaaatgccacagaacctgtccaccaagccacaggacctgtgtactgcccggaacccagacatggaggtggatgagaatggcaccctggacctgagcatgaacaagcagaggcctcgagacagctgctgcccagtcctgacacccctggaacccatgtctccgcagcagcaggccgtgatgagcagccgatgcttccagctgagcgagggggattgctgggacttgcctgtagactacaccaaaatgaagcctcggagggtagatgaggatgagcccaaagagattaccccagaagacttggacccattccaggaggctctggaagaaagacggtatccaggggaggtgaccatcccaagccccaaacccaagtaccctcagtgcaaggaaagcaaaaaggacttaataactctgtctggctgccccctggcggacaaaagcattcgaagtatgctggccaccagttcccaagagctcaagtgccccacccctggctgtgacggttctggacacatcactggcaattacgcttctcatcgaagcctttctgggtgcccgagagcaaagaagagtggcatccggatagcacagagcaaagaggacaaggaagaccaggagccaatcaggtgtccggtacctggctgtgacggtcagggacacatcactgggaagtatgcatcccaccgcagcgcctccgggtgtcccttggcagccaagaggcagaaagatgggtaccttaatggctcccagttctcctggaagtcggtcaagacggagggcatgtcctgccctacccccgggtgtgatgggtcaggacacgtcagtggcagcttcctcacacaccgcagcttgtcaggatgtccaagagccacatcagcaatgaagaaagcaaagctgtctggagaacagatgttgactatcaagcagcgagccagcaacggtatagaaaatgatgaagaaatcaagcagttagatgaagagatcaaggagcttaatgagtccaattcccagatggaggctgacatgatcaaactcagaactcagatcaccacaatggagagcaacctgaagacgattgaggaggagaacaaagtcattgaacagcagaatgagtcgctcttgcacgagttggccaacctgagccagtccctgatccacagcctcgccaacatccagctgcctcacatggatccaatcaatgaacaaaattttgatgcttacgtgactactttgacggaaatgtatacaaatcaagatcgttatcagagtccagaaaataaagccctactggaaaatataaagcaggctgtgagaggaattcaggtctgaacagctgctctagtggtgactcatgcttaaaaaggatgcctcttgtttcttgctgctgtaacttaccagaaagtgttatatatttatttctgtcggaacagtgttatgctacaagacttcataatggttttgtgtgctctcgagagagtacctgcagactagttttggatacattcacattttgtacgttttcatataagctgacatagtgtgatttgccatgtaatgtttatagctgctgctgtctgcacatttgggggtctctatatttctgaagaggtaagctgatgaaaataaatagagtgtaaattctttttaatgctttagtgattaaatgttttagtattttgaactgaaatggacaaaaaaagaaaaaggaaaaaaaaagcaggtttgaacgatcactttgtggcctcctggccacttttagacattatcattttgcctcaggttggaggactttgtggaatttaagaaatacattttgtgtgcatattgtttcatagcaagaattggttgcaaaaaaatgctttatttttgaacaatgcttggaaatattatgtgacttttttgtttgtttgttttaggaggatggtgtatggtgggggcaataaatgaggttttttgcattccaaggaaatggcatatggattaactataagaaatgaaataagtaatttattgtaagacaacatcaagccatggaaacttggcagaagattcaaagcagcttaaacagcacttttaattaactcctaaacattacatggtgggactgtggagactccgttaagacaggagcttgtcagaggtggacaacacgaagatttcctttgcattttcagtaacctttggagcacacttctcttatttctcctagagccccgtgcccctctgaagtgttaccacaatctagcttacctagcagccgttgattgttttttttccatctgagcaaacaggtaatttaagcatttatctcccctttcactttcaaaaagaatcatcattgattattcttgtttcacatctgaggatgaaggctaacggctgtgcttgccctggttactggatgggattctctgctgggcgggtggggaagcagctctacccttccctacccctccctgccccaactctgacttcgaataagccttggtcctatccagaatacactggacaccaaggccaaggacgttcacgttctgccctcgctgggtccagctgactctagcgtctgcacagccttgtgtgactcgtggtgacctaacctgagaaagagtgcaatcagatgttaaagtaactaaatagactgcggcacttttttgctttaaactagatcatcttagatttgtcgataccttcgaaatttgatggtttcatcccaaatgactgcactatatgtatgcatacggccacttttgattgctgcgccccttctgagtagtctttgacaatgtgttgtgttccccgatgtcgacttgatttccttttagtagcatctctctcttccatgtcttgatgttatgcaggaagtacaaaagtactttaaaattttgttatgaaataaaaaaaaaaaggatgggttttgtaaaaataataaaaaaaatatttttagcagaacaggacttacagggtcattgtccccacaatgtgccagtcggctctttgcactcgccttgtcctatatatccgtacggaggtgtgcaatcctgtgtcagtcgccttgtgacactgaagtggatgagttatagaggaggccctcgaggctgacccaatacggttactaagggagactacagggatctcacgacaaacattctgatacaatactcaacctcggtatatatatatatatatatatatatatacatatatatatatgtataaatataagaatatcccagcggcactttatactgttcactgtacaaaagcttacagttttccacaaggactttaataactagctggggaaaagattatgtaattacttggggctctgcaggaccttctctgtccagcgccccctttctgttgtgcgattagttgtagctgccatgctcagaattgccttttgagagctgaagcaaggtgcttactgtcacctgatgccatacacatggtcccaggcccacacccggggggcctctgttcatagcggcacatgcatttccccaccgcgtcttgtctgcagcttcttggccaatgtagtaatgcttttagtagagtaataggtagtatcagtttggattcttattgttatcacctatgtacaatggagaggggttctaagcacaaatctgctgctcatgtaacggtggtacataatatcaaatcaaaagttatctgtgactatatatagggatcacaaagtgtcacatgttagaatgctgaccttccacatggggttattgtgagtcatcagagcatatttattataacttattgttcatattcatttctaagttaatttaagtaatcatttattaagacagaattttgtataaactatttattgtgctctctgtggaactgaagtttgatttatttttgtactacacggcatgggtttgttgacactttaattttgctataaatgtgtggaatcacaagttgctgtgatacttcatttttaaattgtgaactttgtacaaactttgtcatgctgatgtgaacacatcttactctgaataaaaaggtgttgccacgtttgtagcacgaagga

SEQ ID NO: 15 - protein, 244 aa, Murine spp.
MPAPLETCISDLDCSSSNSSSDLSSFLTDEEDCARLQPLASTSGLSVPARRSAPALSGASNVPGAQDEEQERRRRRGRARVRSEALLHSLRRSRRVKANDRERNRMHNLNAALDALRSVLPSFPDDTKLTKIETLRFAYNYIWALAETLRLADQGLPGGSARERLLPPQCVPCLPGPPSPASDTESWGSGAAASPCATVASPLSDPSSPSASEDFTYGPGDPLFSFPGLPKDLLHTTPCFIPYH

SEQ ID NO: 16 - DNA, 1707 bp, Murine spp.
caggagctgatctgatcgccggcgacatcactcaggagaccagcccggcgcgtggcccctgcaggcgaggcgaggagggccaagcccattcactccctgagcccctgcgatcttcccggccctcgcgcctgcagcaggcacaggctagccccgggtcgtacggacagtaagtgcgcttcgaaggccgacctccaaacctcctgtccgtctgtcggtcctgcacactgcaagatgcctgcccctttggagacctgcatctctgatctcgactgctccagcagcaacagcagcagcgacctgtccagcttcctcaccgacgaggaggactgtgccaggctacagcccctagcctccacctcggggctgtccgtgccagcccggaggagcgctcccgccctctccggggcatcgaatgttcccggtgcccaggacgaagagcaggaacggcggaggcggcgaggtcgcgctcgggtgcggtccgaggctctgctgcactccctgcggaggagtcgtcgcgtcaaagccaacgatcgcgagcgcaaccgcatgcacaacctcaacgctgcgctggacgccttgcgcagcgtgctgccctcgttccccgacgacaccaagctcaccaagattgagacgctgcgcttcgcctacaactacatctgggccctggctgagacactgcgcctggcagatcaagggctccccgggggcagtgcccgggagcgcctcctgcctccgcagtgtgtcccctgtctgcccgggcccccgagcccggccagcgacactgagtcctggggttccggggccgctgcctccccctgcgccactgtggcatcaccactctctgaccccagtagtccctcggcttcagaagacttcacctatggcccgggcgatccccttttctcctttcctggcctgcccaaagacctgctccacacgacgccctgtttcatcccataccactaggcctttgtaaggcaacatcaatactttcttcctcccccagtctaagagcaataatagatggggaactggctgaagcctccgggggccacacttacccccaagtgaattctgggagctttaaaggggggagggggaatacctgaccacttgttaggttgctgcaccctcgctgaagctgccctcggtctatttctccacccccagcacggcctccccccccccccgcccgcccccagacggcctttcgtttttgttgcactttctgaacttcacaaaaccttctttgtgactggctcagaactgaccccagccaccacttcagtgtggtttggaaaagggacagatgagcccctgaagacgaggtgaaaagtcaattttacaatttgtagaactctaatgaagaaaaacgagcatgaaaattcggtttgagccggctgacaatacaatggcaaggcttaaaaaggagccacaaggagtgggcttcatgcattatggatcccgacccccgccactgtgggcttgctccaggaagaacctgagtgctagcatagctattcaggcactggactggagggtactttaatttattcaggatgcttcattcatatgaaaatgtatttttgtacataaagaatttattctattattatgagctatcaaagtttacatttttgtactgcagatgcttcgtgtaaataaaaaaaaaaaaaaaaaaaaaaaaaaaaaaaaaaaaaaaaaaaa

SEQ ID NO: 17 - protein, 359 aa, Murine spp.
MVDIIFHYPFLGAMGDHSKKKPGTAMCVGCGSQIHDQFILRVSPDLEWHAACLKCAECSQYLDETCTCFVRDGKTYCKRDYVRLFGIKCAQCQVGFSSSDLVMRARDSVYHIECFRCSVCSRQLLPGDEFSLREHELLCRADHGLLLERAAAGSPRSPGPLPGARGLHLPDAGSGRQPSLRTHVHKQAEKTTRVRTVLNEKQLHTLRTCYAANPRPDALMKEQLVEMTGLSPRVIRVWFQNKRCKDKKKSILMKQLQQQQHSDKASLQGLTGTPLVAGSPIRHENAVQGSAVEVQTYQPPWKALSEFALQSDLDQPAFQQLVSFSESGSLGNSSGSDVTSLSSQLPDTPNSMVPSPVET

SEQ ID NO: 18 - DNA, 1868 bp, Murine spp.
tgcgtgcgggccggtgcggaatcgctccttcaactccgcggggcaggagttagttagcaaagagcagaggctgggcgctcgaccctggtccttctgtccccggccgcaagctttgctcacatctctctctgcatggtggatattatttttcattatccttttctgggtgctatgggggatcattccaagaagaagcccgggacggccatgtgcgtgggctgcgggagtcagatccacgaccagtttatccttcgcgtgtcgcccgacctcgagtggcacgccgcctgcctcaagtgcgcggagtgcagccagtacctggacgagacgtgcacgtgcttcgtgagagacgggaaaacctactgcaagcgggactacgtcaggctgttcggcatcaagtgtgcccagtgccaggtgggcttcagcagcagtgacctggtgatgcgggcgcgggacagcgtgtaccacatcgagtgcttccgctgctccgtgtgcagccgccagctgctccctggagacgagttctcgctgcgggagcatgagctgctctgccgagctgaccacggcctcctgctggagcgcgctgcggctggcagcccgcgcagccccggcccgctccccggcgcccgcggcctgcatctgccagacgctgggtccggacgacagccctcactgcgcacgcacgtgcacaagcaggcggagaagacaacccgggtacggactgtgctcaacgagaagcaactgcataccctgcggacgtgctacgccgccaatccgcggccagacgcgctcatgaaagagcagctagtagagatgaccggcttgagcccgcgggtcatccgcgtgtggtttcagaacaagcgttgcaaggacaagaagaagtccattctcatgaagcagctacagcagcagcaacacagtgacaaggcgagcctccagggactgactgggacgcctctggtggcaggcagccccatccgccatgagaacgcggtgcagggcagcgcagtcgaggtgcagacgtaccagccgccctggaaagcactcagcgagtttgcactccagagcgacctggaccaacccgcctttcaacagctggtttccttctccgagtcaggctccctaggcaactcttccggtagcgacgtgacctctctgtcctcgcagctcccggacacccccaacagtatggtacctagtccggtggagacgtgagcgaggacccctccctgccagcccgcggacctcgcatgctccctgcatgagacgcacccatgctcaggccattccagctctgaaaaatctccggcccttgtaattattgttgttatttaaagcgagcgggcagaggcacgcgaccgcgacgggacggccagcagcgctggggcgcagcctgcaccgctgagactggagactccggctccaaggacttttttttttctttctcataactagaatttgggacattctgggttagctccgccctcgcctctccctcgctgcgtggggaactgtcactgtctctctgcgccttgctcgcccgggaacccatctgatggggtcctacagggccctaccggtgaggacgcctgctccgaactcgcctgcgctgacccgaggctttgttctggacctggcagcgaggacggaacaattccaagaaacagacataacccccaaagcgcatgactgccggacagagtagaaaccagactgtcttttaaaaagtgttttaaattatcagtcgacggaggacagtgagagcctttgccgaacaaacgtaagttattgttatttattgtgaggcgaatagtgggacgaatattttgatcttaataaaacacaacccgatgcaaaaaaaaaaaaaaaaa

SEQ ID NO: 19 - protein, 301 aa, Murine spp.
MDVLASYSIFQELQLVHDTGYFSALPSLEETWQQTCLELERYLQTEPRRISETFGEDLDCFLHASPPPCIEESFRRLDPLLLPVEATICEKSSAVDILLSRDKLLSETCLSLQPTSSSLDSYTAVNQAQLNAVTSLTPPSSPELSRHLVKTSQTLSAVDGTVTLKLVAKKASLSSVKVGGVAAAAAVTPAGAVKSGQSDSEQGGGGADTCPENKKRVHRCQFNGCRKVYTKSSHLKAHQRTHTGEKPYKCSWEGCEWRFARSDELTRHYRKHTGAKPFKCNHCDRCFSRSDHLALHMKRHI

SEQ ID NO: 20 - DNA, 1344 bp, Murine spp.
gtcagtgagtttgcgttccccttcctgatccacgcgctggagtgattagagccctggaagggaattgttactcccgtgaagtccccttttcctggcagtcatctgcactgtacacgctggatccctctctccgatccaccccactcactcgctcctctctcacctcctctctcacctcctctctctccctcctctctcacctcctctctccctctctcctgcattgattttttttctctcctttttttagttgactgaaacaaaacaaaacaaaagggccactggatgtctgccttcttggggggtgagccagacagactgacaaacaaacagacccagctgagctcgggggagggtttctcctccggttttgcccggcagcagcagcatggacgtgttggctagttatagtatattccaggagctacaacttgtccacgacaccggctacttctcagctttgccatccctggaggagacctggcagcagacatgccttgagttggaacgctacctccagacagaaccccggcggatctcggagacctttggtgaggacttggactgcttcctccacgcttctcctcccccatgcattgaggagagcttccggcgcctagaccccttgctgctcccagtggaagccaccatctgcgagaagagctccgcagtggacattttgctctctcgggacaagttgctatctgagacctgcctcagcctccagccaaccagctcttctctagacagctacacagccgtcaaccaggcccagctcaacgcagtgacctcattaacgccgccctcatcccctgagctcagccgccatctggtcaaaacctcacagactctctcagccgtggatggcacagtgacgttgaaactggtggccaagaaggcttccctcagctcggtgaaggtgggaggggtggcggcggcggcagccgtgacaccagctggagcagttaagagtggacagagcgacagtgagcaaggagggggaggggctgacacatgccctgaaaacaagaagagggtccatcgctgtcagtttaacgggtgccggaaagtttatacaaaaagctcccacttaaaggcccaccagaggactcacacaggtgagaagccttacaagtgctcatgggaaggatgcgagtggcgttttgcacggagcgatgagctcacaaggcactacaggaaacacacaggtgcaaagccctttaagtgtaaccactgcgacaggtgtttctccaggtctgaccatcttgcgctccatatgaagagacatatctaaacaaaacaaaacaagacaaaataaaaacaaacaaaagtaaaaaaaaaaaa